(19) United States
(12) Patent Application Publication
(10) Pub. No.: US 2024/0368158 A1
(43) Pub. Date: Nov. 7, 2024

(54) KINASE MODULATORS, COMPOSITIONS COMPRISING THE KINASE MODULATOR, AND METHODS OF USING THE SAME

(71) Applicant: BIOFRONT LTD, Grand Cayman (KY)

(72) Inventors: Tianwei MA, Beijing (CN); Lichao FANG, Beijing (CN); Feng SHI, Beijing (CN); Wei XUE, Beijing (CN); Miao LIU, Beijing (CN); Zheng HUANG, Beijing (CN)

(21) Appl. No.: 18/684,859

(22) PCT Filed: Aug. 22, 2022
(86) PCT No.: PCT/CN2022/113921

§ 371 Date: Feb. 20, 2024

(30) Foreign Application Priority Data
Aug. 24, 2021 (WO) PCT/CN2021/114322

(51) Int. Cl. C07D 471/04; A61K 31/4545; A61K 31/496; A61K 31/519; A61K 31/5377; A61K 31/706; C07D 519/00; C07H 15/26

(52) U.S. Cl. CPC C07D 471/04; A61K 31/4545; A61K 31/496; A61K 31/519; A61K 31/5377; A61K 31/706; C07D 519/00; C07H 15/26

(57) ABSTRACT

The present disclosure provides compounds of Formula I′, compositions comprising the compound of Formula I′, and methods of using the same, in treating diseases, disorders, or conditions mediated by the inhibition of certain kinases, such as hematopoietic progenitor kinase 1 (HPK1) and/or Fms related receptor tyrosine kinases (FLTs), such as FLT3.

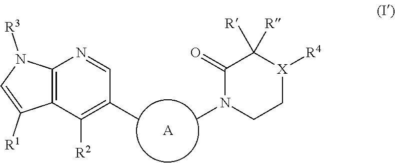

### FIELD OF THE INVENTION

This disclosure relates to novel compounds that are useful for treatment of certain diseases. Specifically, this disclosure relates to compounds that inhibit the activities of certain kinases, for example, hematopoietic progenitor kinases (HPKs), such as HPK1, enhancing an immune response, and/or Fms related receptor tyrosine kinases (FLTs), such as FLT3, and treat certain kinase-dependent disorders, such as HPK1-dependent and/or FLT3-dependent disorders.

### BACKGROUND OF THE INVENTION

Hematopoietic progenitor kinase 1 (HPK1), also known as MAP4K1, is a serine/threonine kinase and is predominantly expressed in hematopoietic cells, such as T cells, B cells and dendritic cells (DC). Structurally, HPK1 comprises N-terminal kinase domain, proline-rich domain, and C-terminal citron homology domain. Traditionally, HPK1 activity can be modulated by kinase domain. HPK1 binds many adaptor proteins, including Grb2, Nck, Crk, SLP-76, and actin-binding adaptors HIP-55. The proline-rich domain can bind proteins that contains SH3 domains. HPK1 can interact with IKK-α/β to prevent complex formation of ADAP and SLP76. HPK1 kinase activity can be induced by a variety of receptor stimulation, including, for example, TCR, BCR, EP2/4, and CD95 (Sawasdikosol & Burakoff, 2020). Upon the TCR engagement, HPK1 is phosphorylated at tyrosine 379 by ZAP70, allowing binding with SH2 domain of SLP76. HPK1 subsequently phosphorylates serine 376 of SLP76 and threonine 262 of Gads (Di Bartolo et al., 2007; Lasserre et al., 2011), creating binding sites for 14-3-3 disruption of SLP76 and LAT complex (di Bartolo et al., 2007; Lasserre et al., 2011). This acts as negative feedback signaling to TCR activation. The functions of HPK1 have been validated by various genetic evidence by either HPK1 deficiency or kinase-dead. HPK1−/− T cells have lower activation threshold with increased pro-inflammatory cytokine and hyper-proliferative response (Liu et al., 2019). HPK1−/− T cells also exhibit resistance to PGE2-mediated suppression (Alzabin et al., 2009). HPK1−/− dendritic cells have demonstrated superior antigen presentation ability in vitro, leading to anti-tumor responses in vivo. In addition, the HPK1−/− mice showed better anti-tumor activity than the wild type mice in several tumor models (Liu et al., 2019). These highlight the importance of HPK1 kinase activity in enhancing immune cells' functions and preventing tumor progression. In addition to autoimmune disease, it was also reported that MAP4K1 expression is a novel resistance mechanism and independent prognostic marker in AML (Knight et al; 2021; Ling et al, 2021). Thus, targeting of HPK1 has the potential to become a novel treatment for cancer and other HPK1-related disorders.

Human Fms-like tyrosine kinase 3 (FLT3), or fetal liver kinase 2 (FLK-2), CD135, is a member of the receptor tyrosine kinases class III. FLT3 is overexpressed in approximately 90% of acute myeloid leukemia (AML), B-precursor cell acute lymphocytic leukemia (ALL), a fraction of T-cell ALL, and the blast-crisis phase of chronic myeloid leukemia (BC-CML). FLT3 is one of the most frequently mutated genes in AML. FLT3 mutations can be subdivided into internal tandem duplicates (ITD), present in approximately 25% of patients, and point mutations (such as D835 and 1836) in the tyrosine kinase domain (TKD), present in approximately 5%. The D835Y substitution constitutes approximately 50% of FLT3-TKD mutations. Both FLT3-ITD and FLT3-TKD mutations are constitutively activating through autophosphorylation, leading to ligand-independent FLT3 signaling and cellular proliferation (Front Oncol, 2020; 10:612880). The current small molecule FLT3 inhibitors did not offer significant clinical benefit as monotherapy. There is a need to provide alternative FLT3 inhibitors which induces rapid down-modulation of FLT3 and downstream kinase of STAT5 pathways in leukemia.

In summary, an HPK1 inhibitor, an FLT3 inhibitor, a dual HPK1-FLT3 inhibitor, or a fixed dose combination of an HPK1 inhibitor and an FLT3 inhibitor may offer more effective treatment options for certain cancer patients.

### SUMMARY OF THE INVENTION

One aspect of the present disclosure provides a compound selected from compounds of Formulae I, I′, IIA, IIA′, IIIA, IIIA′, IIIB, and IIIB′, a tautomer thereof, a deuterated derivative of the compound or the tautomer, or a pharmaceutically acceptable salt of the foregoing, which can be employed in the treatment of diseases mediated by the inhibition of hematopoietic progenitor kinase 1 (HPK1), and/or the inhibition of Human Fms-like tyrosine kinase 3 (FLT3). For example, disclosed herein is a compound of the following structural Formula I:

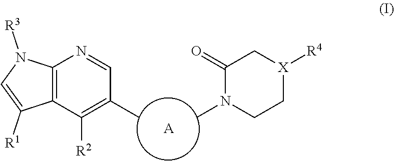

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) R² is chosen from hydrogen, halogen groups, OR^{x}, SR^{x}, NHR^{x}, N(R^{x})₂, CHR^{x}, and C(R^{x})₂;
(iii) R³ is chosen from hydrogen, linear, branched, and cyclic alkyl groups, heterocyclic groups, aryl groups, and heteroaryl groups;
(iv) R⁴ is chosen from hydrogen, linear, branched, and cyclic alkyl groups, heterocyclic groups, C(O)R^{y}, CO₂R^{y}, C(O)R^{w}OR^{y}, C(O)R^{w}N(R^{x}R^{y})₂, OC(O)R^{w}NR^{x}R^{y}, S(O)R^{y}, and SO₂R^{y};
(v) X is chosen from N and CR^{x};
(vi) each R^{x} and R^{y} is independently chosen from hydrogen, linear, branched, and cyclic alkyl groups, carbocyclic groups, heterocyclic groups, aryl groups, and heteroaryl groups;
(vii) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(viii) ring A is chosen from optionally substituted aryls, optionally substituted heteroaryls, optionally substituted cycloalkyls, and optionally substituted heterocycloalkyls;
wherein the linear, branched, and cyclic alkyl groups, linear, branched, and cyclic alkenyl groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups optionally substituted with 0, 1, or 2 C₁-C₆ linear, branched, and cyclic alkyl groups.

In one aspect of the present disclosure, the compounds of Formula I are selected from Compounds 1 to 62 shown below, a tautomer thereof, a deuterated derivative of the compound or the tautomer, or a pharmaceutically acceptable salt of the foregoing.

In some embodiments, the present disclosure provides pharmaceutical compositions comprising a compound of Formulae I, I′, IIA, IIA′, IIIA, IIIA′, HIB, and IIIB′, a tautomer thereof, a deuterated derivative of the compound or the tautomer, or a pharmaceutically acceptable salt of the foregoing, and a pharmaceutically acceptable carrier. In some embodiments, the pharmaceutical compositions comprise a compound selected from Compounds 1 to 62 shown below, a tautomer thereof, a deuterated derivative of the compound or the tautomer, or a pharmaceutically acceptable salt of the foregoing. These compositions may further comprise an additional active pharmaceutical agent.

Another aspect of the present disclosure provides methods of treating a disease, a disorder, or a condition mediated by the inhibition of hematopoietic progenitor kinase 1 (HPK1) and/or the inhibition of Human Fms-like tyrosine kinase 3 in a subject, comprising administering a therapeutically effective amount of a compound of Formulae I, I′, IIA, IIA′, IIA, IIA′, IIIB, and IIIB′, a tautomer thereof, a deuterated derivative of the compound or the tautomer, or a pharmaceutically acceptable salt of the foregoing, or a pharmaceutical composition comprising any of the foregoing. In some embodiments, the methods of treatment comprise administering to a subject, a compound selected from Compounds 1 to 62 shown below, a tautomer thereof, a deuterated derivative of the compound or the tautomer, or a pharmaceutically acceptable salt of the foregoing, or a pharmaceutical composition comprising any of the foregoing.

In some embodiments disclosed herein, the methods of treatment comprise administration of an additional active pharmaceutical agent to the subject in need thereof, either in the same pharmaceutical composition as a compound of Formulae I, I′, IIA, IIA′, IIIA, IIIA′, IIIB, and IIIB′, a tautomer thereof, a deuterated derivative of the compound or the tautomer, or a pharmaceutically acceptable salt of the foregoing, or in a separate composition. In some embodiments disclosed herein, the methods of treatment comprise administering a compound selected from Compounds 1 to 62 shown below, a tautomer thereof, a deuterated derivative of the compound or the tautomer, or a pharmaceutically acceptable salt of the foregoing with an additional active pharmaceutical agent either in the same composition or in a separate composition.

Also disclosed herein are methods of inhibiting HPK1 activities and/or the inhibition of Human Fms-like tyrosine kinase 3 activity, comprising administering to a subject a therapeutically effective amount of a compound of Formulae I, I′, IIA, IIA′, IIIA, IIIA′, IIIB, and IIIB′, a tautomer thereof, a deuterated derivative of the compound or the tautomer, or a pharmaceutically acceptable salt of the foregoing, or a pharmaceutical composition comprising any of the foregoing. In some embodiments disclosed herein, the methods of inhibiting HPK1 and/or the inhibition of Human Fms-like tyrosine kinase 3 activity, comprise administering to a subject, a compound selected from Compounds 1 to 62 shown below, a tautomer thereof, a deuterated derivative of the compound or the tautomer, or a pharmaceutically acceptable salt of the foregoing, or a pharmaceutical composition comprising any of the foregoing. In some embodiments, the methods of inhibiting HPK1 activity and/or the inhibition of Human Fms-like tyrosine kinase 3, comprise contacting said HPK1 and/or Human Fms-like tyrosine kinase 3, with a compound of Formulae I, I′, IIA, IIa′, IIIA, IIIA′, IIIB, and IIIB′, a tautomer thereof, a deuterated derivative of the compound or the tautomer, or a pharmaceutically acceptable salt of the foregoing, or a pharmaceutical composition comprising any of the foregoing. In some embodiments disclosed herein, the methods of inhibiting HPK1 and/or the inhibition of Human Fms-like tyrosine kinase 3, comprise contacting the HPK1 and/or Human Fms-like tyrosine kinase 3, with a compound selected from Compounds 1 to 62 shown below, a tautomer thereof, a deuterated derivative of the compound or the tautomer, or a pharmaceutically acceptable salt of the foregoing, or a pharmaceutical composition comprising any of the foregoing.

### DETAILED DESCRIPTION

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “HPK1” or “hematopoietic progenitor kinase 1” as used herein, also known as MAP4K1, is serine/threonine kinase and is predominantly expressed in hematopoietic cells, such as T cells, B cells and dendritic cells (DC). HPK1 is involved in the modulation of various downstream signaling pathways, such as extracellular signal-regulated kinase (ERK), c-Jun N-terminal kinase (JNK) and nuclear factor-κB (NF-κB) which are all associated with the regulation of cellular proliferation and immune cell activation.

The term “FLT3” or “human Fms-like tyrosine kinase 3” as used herein, is a member of the receptor tyrosine kinases class III. FLT3 is overexpressed in approximately 90% of acute myeloid leukemia (AML), B-precursor cell acute lymphocytic leukemia (ALL), a fraction of T-cell ALL, and the blast-crisis phase of chronic myeloid leukemia (BC-CML). FLT3 is one of the most frequently mutated genes in AML.

Compounds disclosed herein can inhibit HPK1 and/or FLT3. Thus, compounds disclosed herein are generally useful in the treatment of diseases or conditions associated with such kinases. In one embodiment, the compounds disclosed herein are inhibitors, and are useful for treating diseases, such as cancer, associated with such kinase(s).

The term “inhibitor” as used herein means a molecule that inhibits activity of HPK1 and/or FLT3. By “inhibit” herein is meant to decrease the activity of the target enzyme, as compared to the activity of that enzyme in the absence of the inhibitor. In some embodiments, the term “inhibit” means a decrease in HPK1 and/or FLT3 activity of at least about 5%, at least about 10%, at least about 20%, at least about 25%, at least about 50%, at least about 60%, at least about 70%, at least about 80%, at least about 90%, or at least about 95%. In other embodiments, inhibit means a decrease in HPK1 and/or FLT3 activity of about 5% to about 25%, about 25% to about 50%, about 50% to about 75%, or about 75% to 100%. In some embodiments, inhibit means a decrease in HPK1 and/or FLT3 activity of about 95% to 100%, e.g., a decrease in activity of 95%, 96%, 97%, 98%, 99%, or 100%. Such decreases can be measured using a variety of techniques that would be recognizable by one of skill in the art, including in vitro kinase assays.

The term “HPK1 and/or FLT3 inhibitor” as used herein, is a molecule that reduces, inhibits, or otherwise diminishes one or more of the biological activities of HPK1 and/or FLT3. Inhibition using the HPK1 and/or FLT3 inhibitor does not necessarily indicate a total elimination of the HPK1 and/or FLT3 activities. Instead, the activity could decrease by a statistically significant amount, including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of HPK1 and/or FLT3 compared to an appropriate control. In some embodiments, the HPK1 and/or FLT3 inhibitor reduces, inhibits, or otherwise diminishes the serine/threonine kinase activities of HPK1 and/or FLT3. In some of these embodiments, the HPK1 and/or FLT3 inhibitor reduces, inhibits, or otherwise diminishes the HPK1-mediated phosphorylation of SLP76 and/or Gads. The presently disclosed compounds can bind directly to HPK1 and/or FLT3 and inhibit its kinase activity.

The term “compound,” when referring to a compound of the present disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of the present disclosure will depend upon a number of factors, including, for example, the isotopic purity of reagents used to make the compound and the efficiency of incorporation of isotopes in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by the present disclosure are those that result in the formation of stable or chemically feasible compounds.

The term “isotopologue” refers to a species in which the chemical structure differs from only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C are within the scope of the present disclosure.

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the present disclosure. Unless otherwise stated, all tautomeric forms of the compounds of the present disclosure are within the scope of the present disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer” as used herein refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives disclosed herein. Thus, unless otherwise stated, when a reference is made to a “deuterated derivative” of a compound of the present disclosure, at least one hydrogen is replaced with deuterium at a level that is well above its natural isotopic abundance, which is typically about 0.015%. In some embodiments, the deuterated derivatives disclosed herein have an isotopic enrichment factor for each deuterium atom, of at least 3500 (52.5% deuterium incorporation at each designated deuterium), at least 4500 (67.5% deuterium incorporation at each designated deuterium), at least 5000 (75% deuterium incorporation at each designated deuterium), at least 5500 (82.5% deuterium incorporation at each designated deuterium), at least 6000 (90% deuterium incorporation at each designated deuterium), at least 6333.3 (95% deuterium incorporation at each designated deuterium), at least 6466.7 (97% deuterium incorporation at each designated deuterium), or at least 6600 (99% deuterium incorporation at each designated deuterium).

The term “isotopic enrichment factor” as used herein means the ratio between the isotopic abundance and the natural abundance of a specified isotope.

The term “alkyl” as used herein, means a linear or branched, substituted or unsubstituted hydrocarbon chain that is completely saturated. Unless otherwise specified, an alkyl group contains 1 to 30 alkyl carbon atoms. In some embodiments, an alkyl group contains 1 to 20 alkyl carbon atoms. In some embodiments, an alkyl group contains 1 to 10 aliphatic carbon atoms. In some embodiments, an alkyl group contains 1 to 8 aliphatic carbon atoms. In some embodiments, an alkyl group contains 1 to 6 alkyl carbon atoms. In some embodiments, an alkyl group contains 1 to 4 alkyl carbon atoms. In other embodiments, an alkyl group contains 1 to 3 alkyl carbon atoms. And in yet other embodiments, an alkyl group contains 1 to 2 alkyl carbon atoms. In some embodiments, alkyl groups are substituted. In some embodiments, alkyl groups are unsubstituted. In some embodiments, alkyl groups are linear or straight-chain or unbranched. In some embodiments, alkyl groups are branched.

The term “cycloalkyl” refers to a monocyclic C₃₋₈ hydrocarbon or a spirocyclic, fused, or bridged bicyclic or tricyclic C₈₋₁₄ hydrocarbon that is completely saturated, wherein any individual ring in said bicyclic ring system has 3 to 7 members. In some embodiments, cycloalkyl groups are substituted. In some embodiments, cycloalkyl groups are unsubstituted. In some embodiments, the cycloalkyl is a C₃ to C₁₂ cycloalkyl. In some embodiments, the cycloalkyl is a C₃ to C₈ cycloalkyl. In some embodiments, the cycloalkyl is a C₃ to C₆ cycloalkyl. Non-limiting examples of monocyclic cycloalkyls include cyclopropyl, cyclobutyl, cyclopentyl, cyclohexyl, and adamantyl.

The term “carbocyclyl” encompasses the term “cycloalkyl” and refers to a monocyclic C₃₋₈ hydrocarbon or a spirocyclic, fused, or bridged bicyclic or tricyclic C₈₋₁₄ hydrocarbon that is completely saturated, or is partially saturated as it contains one or more units of unsaturation but is not aromatic, wherein any individual ring in said bicyclic ring system has 3 to 7 members. Bicyclic carbocyclyls include combinations of a monocyclic carbocyclic ring fused to, for example, a phenyl. In some embodiments, carbocyclyl groups are substituted. In some embodiments, carbocyclyl groups are unsubstituted. In some embodiments, the carbocyclyl is a C₃ to C₁₂ carbocyclyl. In some embodiments, the carbocyclyl is a C₃ to C₁₀ carbocyclyl. In some embodiments, the carbocyclyl is a C₃ to C₈ carbocyclyl. Non-limiting examples of monocyclic carbocyclyls include cyclopropyl, cyclobutyl, cyclopentanyl, cyclohexyl, cyclopentenyl, cyclohexenyl, etc.

The term “alkenyl” as used herein, means a linear or branched, substituted or unsubstituted hydrocarbon chain that contains one or more double bonds. In some embodiments, alkenyl groups are substituted. In some embodiments, alkenyl groups are unsubstituted. In some embodiments, alkenyl groups are linear, straight-chain, or unbranched. In some embodiments, alkenyl groups are branched.

The term “heterocyclyl” as used herein means non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include, for example, the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic carbocyclyl/cycloalkyl; and a monocyclic heteroaryl fused to a monocyclic carbocyclyl/cycloalkyl. In some embodiments, the “heterocyclyl” group contains 3 to 14 ring members in which one or more ring members is a heteroatom independently chosen, for example, from oxygen, sulfur, nitrogen, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, and phosphorus. In some embodiments, the heterocycle has one heteroatom that is a nitrogen atom. In some embodiments, the heterocycle has one heteroatom that is an oxygen atom. In some embodiments, the heterocycle has two heteroatoms that are each independently selected from nitrogen and oxygen. In some embodiments, the heterocycle has three heteroatoms that are each independently selected from nitrogen and oxygen. In some embodiments, heterocycles are substituted. In some embodiments, heterocycles are unsubstituted. In some embodiments, the heterocyclyl is a 3- to 12-membered heterocyclyl. In some embodiments, the heterocyclyl is a 4- to 10-membered heterocyclyl. In some embodiments, the heterocyclyl is a 3- to 8-membered heterocyclyl. In some embodiments, the heterocyclyl is a 5- to 10-membered heterocyclyl. In some embodiments, the heterocyclyl is a 5- to 8-membered heterocyclyl. In some embodiments, the heterocyclyl is a 5- or 6-membered heterocyclyl. In some embodiments, the heterocyclyl is a 6-membered heterocyclyl. Non-limiting examples of monocyclic heterocyclyls include piperidinyl, piperazinyl, morpholinyl, tetrahydropyranyl, azetidinyl, oxetanyl, tetrahydrothiophenyl, dihyropyranyl, tetrahydropyridinyl, etc.

The term “heteroatom” means one or more of oxygen, sulfur, and nitrogen, including, any oxidized form of nitrogen or sulfur, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains double or triple bonds.

The term “alkoxy” as used herein, refers to an alkyl group, as defined above, wherein one carbon of the alkyl group is replaced by an oxygen (“alkoxy”) atom, provided that the oxygen atom is linked between two carbon atoms.

The term “halogen” includes F, Cl, Br, and I, i.e., fluoro, chloro, bromo, and iodo, respectively.

As used herein, a “cyano” or “nitrile” group refer to —C≡N.

As used herein, a “carboxylate” group refer to —COOH.

As used herein, the term “aminoalkylcarboxylate” group refers to linear or branched, hydrocarbon chain that is completely saturated that is substituted with an amino group and a carboxylate group. In some embodiments, the amino group and the carboxylate group are substituents on the same carbon atom of the alkyl group. In some embodiments, the amino group and the carboxylate group are substituents on different carbon atoms of the alkyl group.

As used herein, an “aromatic ring” refers to a carbocyclic or heterocyclic ring that contains conjugated, planar ring systems with delocalized pi electron orbitals comprised of [4n+2]p orbital electrons, wherein n is an integer of 0 to 6. A “non-aromatic” ring refers to a carbocyclic or heterocyclic that does not meet the requirements set forth above for an aromatic ring, and can be either completely or partially saturated. Nonlimiting examples of aromatic rings include aryl and heteroaryl rings that are further defined as follows.

The term “aryl” used alone or as part of a larger moiety as in “arylalkyl,” “arylalkoxy,” or “aryloxyalkyl,” refers to monocyclic or spirocyclic, fused, or bridged bicyclic or tricyclic ring systems having a total of five to fourteen ring members, wherein every ring in the system is an aromatic ring containing only carbon atoms and wherein each ring in a bicyclic or tricyclic ring system contains 3 to 7 ring members. Nonlimiting examples of aryl groups include phenyl (C₆) and naphthyl (C₁₀) rings. In some embodiments, aryl groups are substituted. In some embodiments, aryl groups are unsubstituted.

The term “heteroaryl” refers to monocyclic or spirocyclic, fused, or bridged bicyclic or tricyclic ring systems having a total of five to fourteen ring members, wherein at least one ring in the system is aromatic, at least one ring in the system contains one or more heteroatoms, and wherein each ring in a bicyclic or tricyclic ring system contains 3 to 7 ring members. Bicyclic heteroaryls include, for example, the following combinations of monocyclic rings: a monocyclic heteroaryl fused to another monocyclic heteroaryl; and a monocyclic heteroaryl fused to a phenyl. In some embodiments, heteroaryl groups are substituted. In some embodiments, heteroaryl groups have one or more heteroatoms chosen, for example, from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, heteroaryl groups are unsubstituted. In some embodiments, the heteroaryl is a 3- to 12-membered heteroaryl. In some embodiments, the heteroaryl is a 3- to 10-membered heteroaryl. In some embodiments, the heteroaryl is a 3- to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. Non-limiting examples of monocyclic heteroaryls are pyridinyl, pyrimidinyl, thiophenyl, thiazolyl, isoxazolyl, 2-amino-4-hydroxy-1H-pteridine, etc.

A “spirocyclic ring system” refers to a ring system having two or more cyclic rings, where every two rings share only one common atom.

The term “glycosidic” refers to a carbohydrate group, such as a monosaccharide, disaccharide, trisaccharide, tetrasaccharide or polysaccharide group, and may exist in various isomeric forms, for example α-D, α-L, β-D or β-L forms. The carbohydrate group may be optionally substituted with other type of substituents or even additional glycosidic groups. In some embodiments, the glycosidic group is selected from α-D-glucopyranoside, α-D-galactopyranoside, α-D-mannopyranoside, α-L-fucopyranoside, α-L-arabinopyranoside, β-D-glucopyranoside, β-D-galactopyranoside, β-D-glucuronide, β-D-lactopyranoside, β-D-xylopyranoside, β-D-glucosaminide, β-D-galactosaminide, β-D-alloside, β-D-lyxoside, β-D-taloside, β-D-threoside, β-D-riboside, β-D-fructoside, β-D-rhamnoside and β-L-guloside groups.

Non-limiting examples of suitable solvents that may be used in the present disclosure include water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in the present disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

Disclosed herein are pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of the present disclosure. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, p-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N+(C₁₋₄alkyl)₄ salts. The present disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The term “subject” refers to an animal, including but not limited to, a human.

The term “therapeutically effective amount” refers to that amount of a compound that produces the desired effect for which it is administered (e.g., improvement in symptoms of diseases, disorders, and conditions mediated by the inhibition of HPK1 and/or FLT3, lessening the severity of diseases, disorders, and conditions mediated by the inhibition of HPK1 and/or FLT3 or a symptom thereof, and/or reducing progression of diseases, disorders, and conditions mediated by the inhibition of HPK1 and/or FLT3 or a symptom thereof). The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, lower risk of diseases, disorders, and conditions mediated by the inhibition of HPK1 and/or FLT3, and disease-related complications. Improvements in or lessening the severity of any of these symptoms can be readily assessed according to methods and techniques known in the art or subsequently developed.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

### II. Compounds and Compositions

In a first embodiment, a compound of the present disclosure is a compound of the following structural formula I:

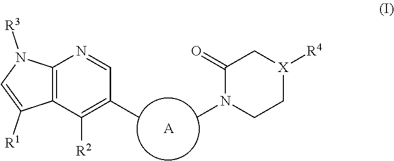

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) R² is chosen from hydrogen, halogen groups, OR^{x}, SR^{x}, NHR^{x}, N(R^{x})₂, CHR^{x}, and C(R^{x})₂;
(iii) R³ is chosen from hydrogen, linear, branched, and cyclic alkyl groups, heterocyclic groups, aryl groups, and heteroaryl groups;
(iv) R⁴ is chosen from hydrogen, linear, branched, and cyclic alkyl groups, heterocyclic groups, C(O)R^{y}, CO₂R^{y}, C(O)R^{w}OR^{y}, C(O)R^{w}N(R^{x}R^{y})₂, OC(O)R^{w}NR^{x}R^{y}, S(O)R^{y}, and SO₂R^{y};
(v) X is chosen from N and CR^{x};
(vi) each R^{x} and R^{y} is independently chosen from hydrogen, linear, branched, and cyclic alkyl groups, carbocyclic groups, heterocyclic groups, aryl groups, and heteroaryl groups;
(vii) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(viii) ring A is chosen from optionally substituted aryls, optionally substituted heteroaryls, optionally substituted cycloalkyls, and optionally substituted heterocycloalkyls;
wherein the linear, branched, and cyclic alkyl groups, linear, branched, and cyclic alkenyl groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups optionally substituted with 0, 1, or 2 C₁-C₆ linear, branched, and cyclic alkyl groups.

In a second embodiment, a compound of the present disclosure is a compound of the following structural formula I′:

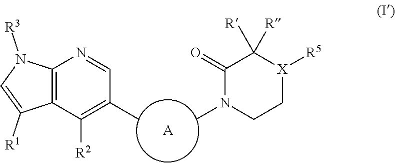

a tautomer thereof, a deuterated derivative of the compound or the tautomer, or a pharmaceutically acceptable salt the foregoing, wherein:
(ix) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(x) R² is chosen from hydrogen, halogen groups, OR^{x}, SR^{x}, NHR^{x}, N(R^{x})₂, CHR^{x}, and C(R^{x})₂;
(xi) R³ is chosen from hydrogen, linear, branched, and cyclic alkyl groups, heterocyclic groups, aryl groups, and heteroaryl groups;
(xii) R⁵ is chosen from hydrogen, linear, branched, and cyclic alkyl groups, heterocyclic groups, C(O)R^{y}, CO₂R^{y}, C(O)R^{w}OR^{y}, C(O)R^{w}NC(O)R^{x}, C(O)R^{w}NC(O)R^{w}NHR^{y}, C(O)R^{w}NR^{x}R^{y}, OC(O)R^{w}NR^{x}R^{y}, S(O)R^{y}, and SO₂R^{y};
(xiii) each R′ and R″ is independently chosen from hydrogen, linear, branched, and cyclic alkyl groups, linear, branched, and cyclic aminoalkyl groups, carbocyclic groups, heterocyclic groups;
(xiv) X is chosen from N and CR^{x};
(xv) each R^{x} and R^{y} is independently chosen from hydrogen, linear, branched, and cyclic alkyl groups, linear, branched, and cyclic aminoalkyl groups, carbocyclic groups, heterocyclic groups, aryl groups, heteroaryl groups, and a glycosidic group;
(xvi) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(xvii) ring A is chosen from optionally substituted aryls, optionally substituted heteroaryls, optionally substituted cycloalkyls, and optionally substituted heterocycloalkyls;
wherein the linear, branched, and cyclic alkyl groups, linear, branched, and cyclic alkenyl groups, carbocyclic groups, linear, branched, and cyclic aminoalkyl groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   carboxylate group;
   hydroxy,
   thiol,
   amino,
   cyano,

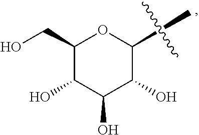

—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic aminoalkylcarboxylate groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups optionally substituted with 0, 1, or 2 C₁-C₆ linear, branched, and cyclic alkyl groups or halogen groups, and
5 and 6-membered heteroaryl groups optionally substituted with 0, 1, or 2 C₁-C₆ linear, branched, and cyclic alkyl groups.

In a second embodiment, a compound of the present disclosure is of one of the following structural formula IIA:

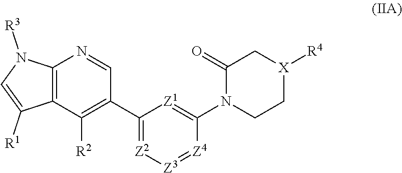

a tautomer thereof, a deuterated derivative of the compound or the tautomer, or a pharmaceutically acceptable salt of the foregoing; wherein each Z¹, Z², Z³, and Z⁴ is independently chosen from CR^{z} and N, wherein R^{z} is independently chosen from hydrogen, linear, branched, and cyclic alkyl groups, carbocyclic groups, heterocyclic groups, aryl groups, and heteroaryl groups; and all other variables not specifically defined herein are as defined in the first embodiment.

In a third embodiment, a compound of the present disclosure is of one of the following structural formula IIA′:

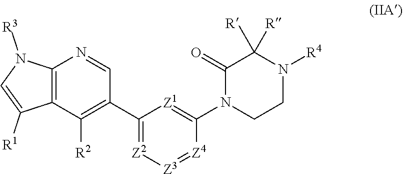

a tautomer thereof, a deuterated derivative of the compound or the tautomer, or a pharmaceutically acceptable salt of the foregoing; wherein each Z¹, Z², Z³, and Z⁴ is independently chosen from CR^{z} and N, wherein R^{z} is independently chosen from hydrogen, linear, branched, and cyclic alkyl groups, carbocyclic groups, heterocyclic groups, aryl groups, and heteroaryl groups; and all other variables not specifically defined herein are as defined in the second embodiment in formula (I′).

In a fourth embodiment, a compound of the present disclosure is of one of the following structural formula IIIA:

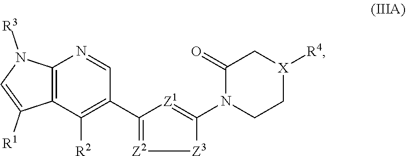

a tautomer thereof, a deuterated derivative of the compound or the tautomer, or a pharmaceutically acceptable salt of the foregoing; wherein each Z¹ and Z² is independently chosen from CR^{z} and N, Z³ is chosen from O, S, and NR^{z}, R^{z} is independently chosen from hydrogen, linear, branched, and cyclic alkyl groups, carbocyclic groups, heterocyclic groups, aryl groups, and heteroaryl groups and all other variables not specifically defined herein are as defined in the first embodiment.

In a fifth embodiment, a compound of the present disclosure is of one of the following structural formula IIIA′:

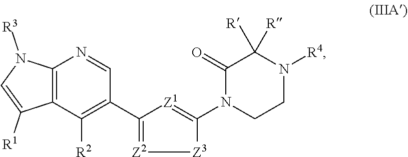

a tautomer thereof, a deuterated derivative of the compound or the tautomer, or a pharmaceutically acceptable salt of the foregoing; wherein each Z¹ and Z² is independently chosen from CR^{z} and N, Z³ is chosen from O, S, and NR^{z}; wherein R^{z} is independently chosen from hydrogen, linear, branched, and cyclic alkyl groups, carbocyclic groups, heterocyclic groups, aryl groups, and heteroaryl groups and all other variables not specifically defined herein are as defined in the second embodiment in formula (I′).

In a sixth embodiment, a compound of the present disclosure is of one of the following structural formula IIIB:

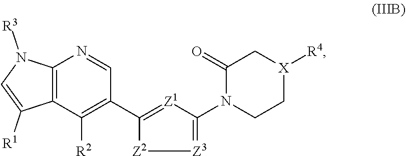

a tautomer thereof, a deuterated derivative of the compound or the tautomer, or a pharmaceutically acceptable salt of the foregoing; wherein each Z¹ and Z³ is independently chosen from CR^{z} and N, Z² is chosen from O, S, and NR^{z}; wherein R^{z} is independently chosen from hydrogen, linear, branched, and cyclic alkyl groups, carbocyclic groups, heterocyclic groups, aryl groups, and heteroaryl groups and all other variables not specifically defined herein are as defined in the first embodiment.

In a seventh embodiment, a compound of the present disclosure is of one of the following structural formula IIIB′:

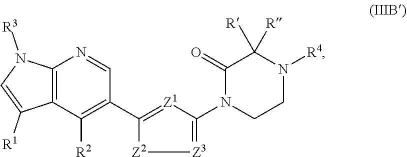

a tautomer thereof, a deuterated derivative of the compound or the tautomer, or a pharmaceutically acceptable salt of the foregoing; wherein each Z¹ and Z³ is independently chosen from CR^{z} and N, Z² is chosen from O, S, and NR^{z}; wherein R^{z} is independently chosen from hydrogen, linear, branched, and cyclic alkyl groups, carbocyclic groups, heterocyclic groups, aryl groups, and heteroaryl groups and all other variables not specifically defined herein are as defined in the second embodiment in formula (I′).

In a fifth embodiment, in a compound, tautomer, deuterated derivative, or pharmaceutically acceptable salt of the present disclosure, R¹ is chosen from linear, branched, and cyclic alkyl groups; R² is a halogen group; and R³ is chosen from hydrogen, linear, branched, and cyclic alkyl groups; and all other variables not specifically defined herein are as defined in any one of the first, second, third, and fourth embodiments.

In a sixth embodiment, in a compound, tautomer, deuterated derivative, or pharmaceutically acceptable salt of the present disclosure, R¹ is chosen from C₁-C₆ linear, branched, and cyclic alkyl groups; and all other variables not specifically defined herein are as defined in any one of the first, second, third, and fourth embodiments.

In a seventh embodiment, in a compound, tautomer, deuterated derivative, or pharmaceutically acceptable salt of the present disclosure, R¹ is chosen from methyl, ethyl, cyclopropyl, and cyclobutyl; and all other variables not specifically defined herein are as defined in any one of the first, second, third, and fourth embodiments.

In an eighth embodiment, in a compound, tautomer, deuterated derivative, or pharmaceutically acceptable salt of the present disclosure, R¹ is chosen from heterocyclic group; and all other variables not specifically defined herein are as defined in any one of the first, second, third, and fourth embodiments.

In a ninth embodiment, in a compound, tautomer, deuterated derivative, or pharmaceutically acceptable salt of the present disclosure, R¹ is chosen from linear, branched, and cyclic alkynyl groups; and all other variables not specifically defined herein are as defined in any one of the first, second, third, and fourth embodiments.

In a tenth embodiment, in a compound, tautomer, deuterated derivative, or pharmaceutically acceptable salt of the present disclosure, R¹ is chosen from linear, branched, and cyclic alkynyl groups substituted with at least one group chosen from C₁-C₆ linear, branched, and cyclic alkyl groups, C₁-C₆ linear, branched, and cyclic aminoalkyl groups, 3 to 6-membered heterocyclic groups, and 5 and 6-membered heteroaryl groups; and all other variables not specifically defined herein are as defined in any one of the first, second, third, and fourth embodiments.

In an eleventh embodiment, in a compound, tautomer, deuterated derivative, or pharmaceutically acceptable salt of the present disclosure, R² is a halogen group; and all other variables not specifically defined herein are as defined in any one of the first, second, third, and fourth embodiments.

In a twelfth embodiment, in a compound, tautomer, deuterated derivative, or pharmaceutically acceptable salt of the present disclosure, R² is fluoro; and all other variables not specifically defined herein are as defined in any one of the first, second, third, and fourth embodiments.

In a thirteenth embodiment, in a compound, tautomer, deuterated derivative, or pharmaceutically acceptable salt of the present disclosure, R² is chloro; and all other variables not specifically defined herein are as defined in any one of the first, second, third, and fourth embodiments.

In a fourteenth embodiment, in a compound, tautomer, deuterated derivative, or pharmaceutically acceptable salt of the present disclosure, R² is hydrogen; and all other variables not specifically defined herein are as defined in any one of the first, second, third, and fourth embodiments.

In a fifteenth embodiment, in a compound, tautomer, deuterated derivative, or pharmaceutically acceptable salt of the present disclosure, R² is chosen from linear, branched, and cyclic alkyl groups; and all other variables not specifically defined herein are as defined in any one of the first, second, third, and fourth embodiments.

In a sixteenth embodiment, in a compound, tautomer, deuterated derivative, or pharmaceutically acceptable salt of the present disclosure, R² is chosen from methyl, ethyl, n-propyl, iso-propyl, cyclopropyl, n-butyl, iso-butyl, sec-butyl, tert-butyl, and cyclobutyl; and all other variables not specifically defined herein are as defined in any one of the first, second, third, and fourth embodiments.

In a seventeenth embodiment, in a compound, tautomer, deuterated derivative, or pharmaceutically acceptable salt of the present disclosure, R⁴ is hydrogen; and all other variables not specifically defined herein are as defined in any one of the first, second, third, and fourth embodiments.

In an eighteenth embodiment, in a compound, tautomer, deuterated derivative, or pharmaceutically acceptable salt of the present disclosure, R⁴ is CO₂R^{y}, wherein R^{y} is chosen from linear, branched, and cyclic alkyl groups; and all other variables not specifically defined herein are as defined in any one of the first, second, third, and fourth embodiments.

In a nineteenth embodiment, in a compound, tautomer, deuterated derivative, or pharmaceutically acceptable salt of the present disclosure, R⁴ is C(O)R^{y}, wherein R^{y} is chosen from linear, branched, and cyclic alkyl groups; and all other variables not specifically defined herein are as defined in any one of the first, second, third, and fourth embodiments.

In a twentieth embodiment, in a compound, tautomer, deuterated derivative, or pharmaceutically acceptable salt of the present disclosure, R^{y} is chosen from C₁-C₆ linear alkyl groups; and all other variables not specifically defined herein are as defined in any one of the first, second, third, and fourth embodiments.

In a twenty-first embodiment, in a compound, tautomer, deuterated derivative, or pharmaceutically acceptable salt of the present disclosure, R^{y} is chosen from C₁-C₆ linear alkyl groups substituted with —N(C₁-C₆ linear, branched, and cyclic alkyl groups)₂; and all other variables not specifically defined herein are as defined in any one of the first, second, third, and fourth embodiments.

In a twenty-second embodiment, in a compound, tautomer, deuterated derivative, or pharmaceutically acceptable salt of the present disclosure, R^{y} is chosen from C₁-C₆ linear alkyl groups substituted with C₁-C₆ linear, branched, and cyclic hydroxyalkyl groups; and all other variables not specifically defined herein are as defined in any one of the first, second, third, and fourth embodiments.

In a twenty-third embodiment, in a compound, tautomer, deuterated derivative, or pharmaceutically acceptable salt of the present disclosure, R^{y} is chosen from C₁-C₆ linear alkyl groups substituted with C₁-C₆ linear, branched, and cyclic aminoalkyl groups; and all other variables not specifically defined herein are as defined in any one of the first, second, third, and fourth embodiments.

In a twenty-fourth embodiment, in a compound, tautomer, deuterated derivative, or pharmaceutically acceptable salt of the present disclosure, R^{y} is chosen from C₁-C₆ linear alkyl groups substituted with C₁-C₆ linear, branched, and cyclic alkoxy groups; and all other variables not specifically defined herein are as defined in any one of the first, second, third, and fourth embodiments.

In a twenty-fifth embodiment, in a compound, tautomer, deuterated derivative, or pharmaceutically acceptable salt of the present disclosure, R⁴ is SO₂R^{y}, wherein R^{y} is chosen from linear, branched, and cyclic alkyl groups; and all other variables not specifically defined herein are as defined in any one of the first, second, third, and fourth embodiments.

In a twenty-sixth embodiment, in a compound, tautomer, deuterated derivative, or pharmaceutically acceptable salt of the present disclosure; R⁴ is SO₂R^{y}, wherein R^{y} is chosen C₁-C₆ linear alkyl groups; and all other variables not specifically defined herein are as defined in any one of the first, second, third, and fourth embodiments.

In a twenty-seventh embodiment, in a compound, tautomer, deuterated derivative, or pharmaceutically acceptable salt of the present disclosure, ring A is chosen from aryl groups; and all other variables not specifically defined herein are as defined in any one of the first, second, third, and fourth embodiments.

In a twenty-eighth embodiment, in a compound, tautomer, deuterated derivative, or pharmaceutically acceptable salt of the present disclosure, ring A is phenyl; and all other variables not specifically defined herein are as defined in any one of the first, second, third, and fourth embodiments.

In a twenty-ninth embodiment, in a compound, tautomer, deuterated derivative, or pharmaceutically acceptable salt of the present disclosure, ring A is chosen from aryl groups, wherein the aryl group is substituted with halogen groups; and all other variables not specifically defined herein are as defined in any one of the first, second, third, and fourth embodiments.

In a thirtieth embodiment, in a compound, tautomer, deuterated derivative, or pharmaceutically acceptable salt of the present disclosure, ring A is chosen from aryl groups, wherein the aryl group is substituted with C₁-C₆ linear, branched, and cyclic alkyl groups; and all other variables not specifically defined herein are as defined in any one of the first, second, third, and fourth embodiments.

In a thirty-first embodiment, in a compound, tautomer, deuterated derivative, or pharmaceutically acceptable salt of the present disclosure, ring A is chosen from heteroaryl groups; and all other variables not specifically defined herein are as defined in any one of the first, second, third, and fourth embodiments.

In a thirty-second embodiment, in a compound, tautomer, deuterated derivative, or pharmaceutically acceptable salt of the present disclosure, ring A is chosen from heteroaryl groups, wherein the heteroaryl group is substituted with halogen groups; and all other variables not specifically defined herein are as defined in any one of the first, second, third, and fourth embodiments.

In a thirty-third embodiment, in a compound, tautomer, deuterated derivative, or pharmaceutically acceptable salt of the present disclosure, ring A is chosen from heteroaryl groups, wherein the heteroaryl group is substituted with C₁-C₆ linear, branched, and cyclic alkyl groups; and all other variables not specifically defined herein are as defined in any one of the first, second, third, and fourth embodiments.

In a thirty-fourth embodiment, in a compound, tautomer, deuterated derivative, or pharmaceutically acceptable salt of the present disclosure, ring A is chosen from 6-membered heteroaryl groups; and all other variables not specifically defined herein are as defined in any one of the first, second, third, and fourth embodiments.

In a thirty-fifth embodiment, in a compound, tautomer, deuterated derivative, or pharmaceutically acceptable salt of the present disclosure, ring A is chosen from 6-membered heteroaryl groups, wherein the 6-membered heteroaryl groups is substituted with halogen groups; and all other variables not specifically defined herein are as defined in any one of the first, second, third, and fourth embodiments.

In a thirty-sixth embodiment, in a compound, tautomer, deuterated derivative, or pharmaceutically acceptable salt of the present disclosure, ring A is chosen from 6-membered heteroaryl groups, wherein the 6-membered heteroaryl groups is substituted with C₁-C₆ linear, branched, and cyclic alkyl groups; and all other variables not specifically defined herein are as defined in any one of the first, second, third, and fourth embodiments.

In a thirty-seventh embodiment, in a compound, tautomer, deuterated derivative, or pharmaceutically acceptable salt of the present disclosure, ring A is a pyridine ring; and all other variables not specifically defined herein are as defined in any one of the first, second, third, and fourth embodiments.

In a thirty-eighth embodiment, in a compound, tautomer, deuterated derivative, or pharmaceutically acceptable salt of the present disclosure, ring A is a pyrimidine ring; and all other variables not specifically defined herein are as defined in any one of the first, second, third, and fourth embodiments.

In a thirty-ninth embodiment, in a compound, tautomer, deuterated derivative, or pharmaceutically acceptable salt of the present disclosure, ring A is chosen from 5-membered heteroaryl groups; and all other variables not specifically defined herein are as defined in any one of the first, second, third, and fourth embodiments.

In a fortieth embodiment, in a compound, tautomer, deuterated derivative, or pharmaceutically acceptable salt of the present disclosure, ring A is chosen from 5-membered heteroaryl groups, wherein the 5-membered heteroaryl groups is substituted with halogen groups; and all other variables not specifically defined herein are as defined in any one of the first, second, third, and fourth embodiments.

In a forty-first embodiment, in a compound, tautomer, deuterated derivative, or pharmaceutically acceptable salt of the present disclosure, ring A is chosen from 5-membered heteroaryl groups, wherein the 5-membered heteroaryl groups is substituted with C₁-C₆ linear, branched, and cyclic alkyl groups; and all other variables not specifically defined herein are as defined in any one of the first, second, third, and fourth embodiments.

In a forty-second embodiment, in a compound, tautomer, deuterated derivative, or pharmaceutically acceptable salt of the present disclosure, ring A is a thiazole ring; and all other variables not specifically defined herein are as defined in any one of the first, second, third, and fourth embodiments.

In a forty-third embodiment, in a compound, tautomer, deuterated derivative, or pharmaceutically acceptable salt of the present disclosure, Z¹ is N, Z² is CR^{z}, Z³ is CR^{z}, and Z⁴ is CR^{z}; and all other variables not specifically defined herein are as defined in any one of the first and second embodiments.

In a forty-fourth embodiment, in a compound, tautomer, deuterated derivative, or pharmaceutically acceptable salt of the present disclosure, Z¹ is CR^{z}, Z² is N, Z³ is CR^{z}, and Z⁴ is CR^{z}; and all other variables not specifically defined herein are as defined in any one of the first and second embodiments.

In a forty-fifth embodiment, in a compound, tautomer, deuterated derivative, or pharmaceutically acceptable salt of the present disclosure, Z¹ is CR^{z}, Z² is CR^{z}, Z³ is N, and Z⁴ is CR^{z}; and all other variables not specifically defined herein are as defined in any one of the first and second embodiments.

In a fourth-sixth embodiment, in a compound, tautomer, deuterated derivative, or pharmaceutically acceptable salt of the present disclosure, Z¹ is CR^{z}, Z² is CR^{z}, Z³ is CR^{z}, and Z⁴ is N; and all other variables not specifically defined herein are as defined in any one of the first and second embodiments.

In a forty-seventh embodiment, in a compound, tautomer, deuterated derivative, or pharmaceutically acceptable salt of the present disclosure, Z¹ is N, Z² is N, Z³ is CR^{z}, and Z⁴ is CR^{z}; and all other variables not specifically defined herein are as defined in any one of the first and second embodiments.

In a forty-eighth embodiment, in a compound, tautomer, deuterated derivative, or pharmaceutically acceptable salt of the present disclosure, Z¹ is N, Z² is CR^{z}, Z³ is N, and Z⁴ is CR^{z}; and all other variables not specifically defined herein are as defined in any one of the first and second embodiments.

In a forty-ninth embodiment, in a compound, tautomer, deuterated derivative, or pharmaceutically acceptable salt of the present disclosure, Z¹ is CR^{z}, Z² is CR^{z}, Z³ is CR^{z}, and Z⁴ is N; and all other variables not specifically defined herein are as defined in any one of the first and second embodiments.

In a fiftieth embodiment, in a compound, tautomer, deuterated derivative, or pharmaceutically acceptable salt of the present disclosure, Z¹ is N, Z² is CR^{z}, Z³ is CR^{z}, and Z⁴ is N; and all other variables not specifically defined herein are as defined in any one of the first and second embodiments.

In a fifty-first embodiment, in a compound, tautomer, deuterated derivative, or pharmaceutically acceptable salt of the present disclosure, Z¹ is CR^{z}, Z² is CR^{z}, and Z³ is O; and all other variables not specifically defined herein are as defined in any one of the first and third embodiments.

In a fifty-second embodiment, in a compound, tautomer, deuterated derivative, or pharmaceutically acceptable salt of the present disclosure, Z¹ is CR^{z}, Z² is CR^{z}, and Z³ is S; and all other variables not specifically defined herein are as defined in any one of the first and third embodiments.

In a fifty-third embodiment, in a compound, tautomer, deuterated derivative, or pharmaceutically acceptable salt of the present disclosure, Z¹ is N, Z² is CR^{z}, and Z³ is O; and all other variables not specifically defined herein are as defined in any one of the first and third embodiments.

In a fifty-fourth embodiment, in a compound, tautomer, deuterated derivative, or pharmaceutically acceptable salt of the present disclosure, Z¹ is N, Z² is CR^{z}, and Z³ is S; and all other variables not specifically defined herein are as defined in any one of the first and third embodiments.

In a fifty-fifth embodiment, in a compound, tautomer, deuterated derivative, or pharmaceutically acceptable salt of the present disclosure, Z¹ is CR^{z}, Z² is N, and Z³ is O; and all other variables not specifically defined herein are as defined in any one of the first and third embodiments.

In a fifty-sixth embodiment, in a compound, tautomer, deuterated derivative, or pharmaceutically acceptable salt of the present disclosure, Z¹ is CR^{z}, Z² is N, and Z³ is S; and all other variables not specifically defined herein are as defined in any one of the first and third embodiments.

In a fifty-seventh embodiment, in a compound, tautomer, deuterated derivative, or pharmaceutically acceptable salt of the present disclosure, Z¹ is CR^{z}, Z² is O, and Z³ is CR^{z}; and all other variables not specifically defined herein are as defined in any one of the first and fourth embodiments.

In a fifty-eighth embodiment, in a compound, tautomer, deuterated derivative, or pharmaceutically acceptable salt of the present disclosure, Z¹ is CR^{z}, Z² is S, and Z³ is CR^{z}; and all other variables not specifically defined herein are as defined in any one of the first and fourth embodiments.

In a fifty-ninth embodiment, in a compound, tautomer, deuterated derivative, or pharmaceutically acceptable salt of the present disclosure, Z¹ is N, Z² is O, and Z³ is CR^{z}; and all other variables not specifically defined herein are as defined in any one of the first and fourth embodiments.

In a sixtieth embodiment, in a compound, tautomer, deuterated derivative, or pharmaceutically acceptable salt of the present disclosure, Z¹ is N, Z² is S, and Z³ is CR^{z}; and all other variables not specifically defined herein are as defined in any one of the first and fourth embodiments.

In a sixty-first embodiment, in a compound, tautomer, deuterated derivative, or pharmaceutically acceptable salt of the present disclosure, Z¹ is CR^{z}, Z² is O, and Z³ is N; and all other variables not specifically defined herein are as defined in any one of the first and fourth embodiments.

In a sixty-second embodiment, in a compound, tautomer, deuterated derivative, or pharmaceutically acceptable salt of the present disclosure, Z¹ is CR^{z}, Z² is S, and Z³ is N; and all other variables not specifically defined herein are as defined in any one of the first and fourth embodiments.

In certain embodiments, the at least one compound of the present disclosure is selected from Compounds 1 to 62 shown in Table 1 below, a tautomer thereof, a deuterated derivative of the compound or the tautomer, or a pharmaceutically acceptable salt of the

TABLE 1

| | |
|---|---|
| 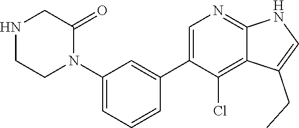 [SMILES: CCc1c[nH]c2ncc(-c3cccc(N4CCNCC4=O)c3)c(Cl)c12] | 1 |
| 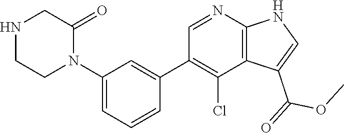 [SMILES: COC(=O)c1c[nH]c2ncc(-c3cccc(N4CCNCC4=O)c3)c(Cl)c12] | 2 |
| 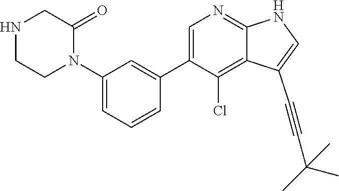 [SMILES: CC(C)(C)C#Cc1c[nH]c2ncc(-c3cccc(N4CCNCC4=O)c3)c(Cl)c12] | 3 |
| 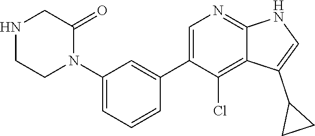 [SMILES: O=C1CNCCN1c1cccc(-c2cnc3[nH]cc(C4CC4)c3c2Cl)c1] | 4 |
| 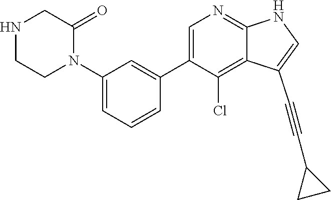 [SMILES: O=C1CNCCN1c1cccc(-c2cnc3[nH]cc(C#CC4CC4)c3c2Cl)c1] | 5 |
| 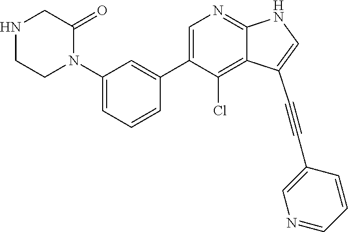 [SMILES: O=C1CNCCN1c1cccc(-c2cnc3[nH]cc(C#Cc4cccnc4)c3c2Cl)c1] | 6 |
| 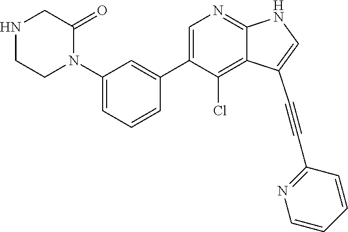 [SMILES: O=C1CNCCN1c1cccc(-c2cnc3[nH]cc(C#Cc4ccccn4)c3c2Cl)c1] | 7 |
| 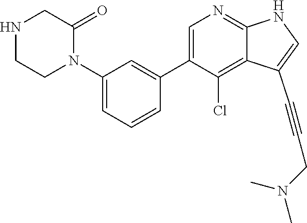 [SMILES: CN(C)CC#Cc1c[nH]c2ncc(-c3cccc(N4CCNCC4=O)c3)c(Cl)c12] | 8 |
| 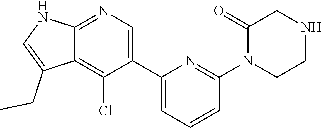 [SMILES: CCc1c[nH]c2ncc(-c3cccc(N4CCNCC4=O)n3)c(Cl)c12] | 9 |
| 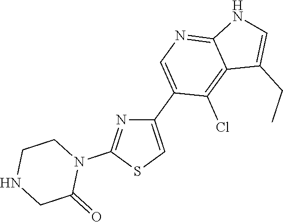 [SMILES: CCc1c[nH]c2ncc(-c3csc(N4CCNCC4=O)n3)c(Cl)c12] | 10 |
| 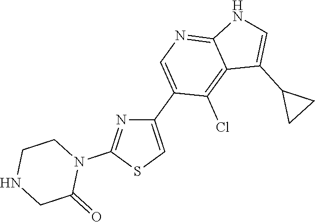 [SMILES: O=C1CNCCN1c1nc(-c2cnc3[nH]cc(C4CC4)c3c2Cl)cs1] | 11 |
| 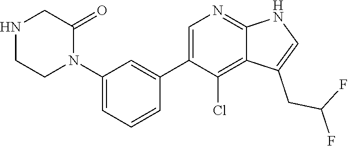 [SMILES: O=C1CNCCN1c1cccc(-c2cnc3[nH]cc(CC(F)F)c3c2Cl)c1] | 12 |
| 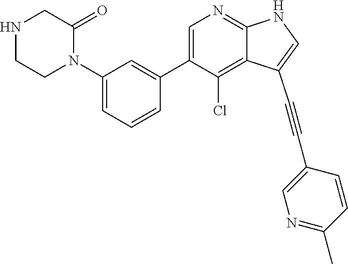 [SMILES: Cc1ccc(C#Cc2c[nH]c3ncc(-c4cccc(N5CCNCC5=O)c4)c(Cl)c23)cn1] | 13 |
| 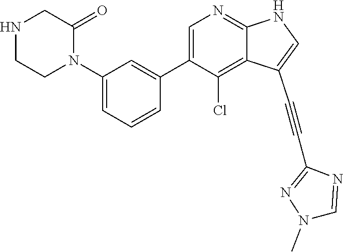 [SMILES: Cn1cnc(C#Cc2c[nH]c3ncc(-c4cccc(N5CCNCC5=O)c4)c(Cl)c23)n1] | 14 |
| 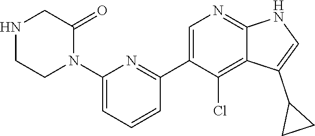 [SMILES: O=C1CNCCN1c1cccc(-c2cnc3[nH]cc(C4CC4)c3c2Cl)n1] | 15 |
| 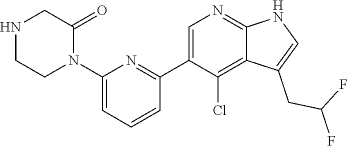 [SMILES: O=C1CNCCN1c1cccc(-c2cnc3[nH]cc(CC(F)F)c3c2Cl)n1] | 16 |
| 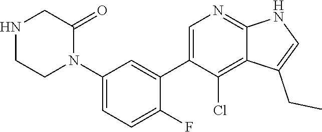 [SMILES: CCc1c[nH]c2ncc(-c3cc(N4CCNCC4=O)ccc3F)c(Cl)c12] | 17 |
| 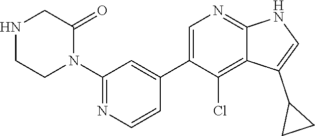 [SMILES: O=C1CNCCN1c1cc(-c2cnc3[nH]cc(C4CC4)c3c2Cl)ccn1] | 18 |
| 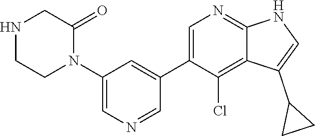 [SMILES: O=C1CNCCN1c1cncc(-c2cnc3[nH]cc(C4CC4)c3c2Cl)c1] | 19 |
| 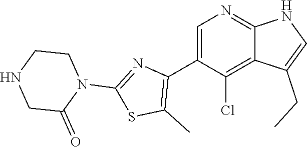 [SMILES: CCc1c[nH]c2ncc(-c3nc(N4CCNCC4=O)sc3C)c(Cl)c12] | 20 |
| 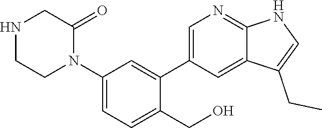 [SMILES: CCc1c[nH]c2ncc(-c3cc(N4CCNCC4=O)ccc3CO)cc12] | 21 |
| 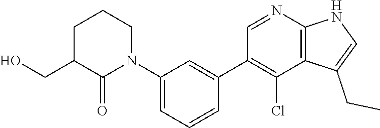 [SMILES: CCc1c[nH]c2ncc(-c3cccc(N4CCCC(CO)C4=O)c3)c(Cl)c12] | 22 |
| 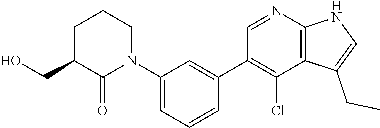 [SMILES: CCc1c[nH]c2ncc(-c3cccc(N4CCC[C@H](CO)C4=O)c3)c(Cl)c12] | 23 |
| 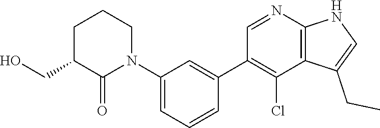 [SMILES: CCc1c[nH]c2ncc(-c3cccc(N4CCC[C@@H](CO)C4=O)c3)c(Cl)c12] | 24 |
| 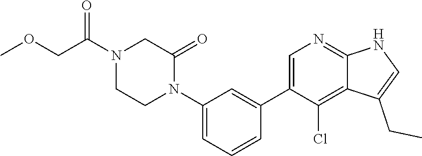 [SMILES: CCc1c[nH]c2ncc(-c3cccc(N4CCN(C(=O)COC)CC4=O)c3)c(Cl)c12] | 25 |
| 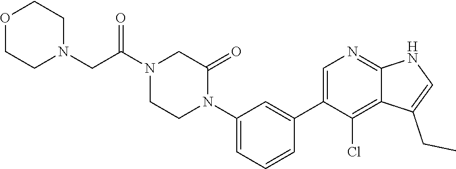 [SMILES: CCc1c[nH]c2ncc(-c3cccc(N4CCN(C(=O)CN5CCOCC5)CC4=O)c3)c(Cl)c12] | 26 |
| 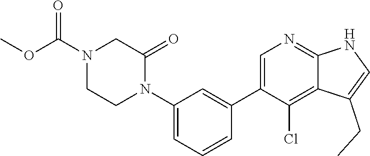 [SMILES: CCc1c[nH]c2ncc(-c3cccc(N4CCN(C(=O)OC)CC4=O)c3)c(Cl)c12] | 27 |
| 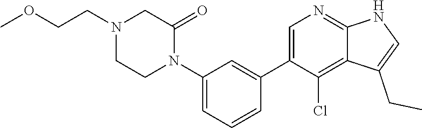 [SMILES: CCc1c[nH]c2ncc(-c3cccc(N4CCN(CCOC)CC4=O)c3)c(Cl)c12] | 28 |
| 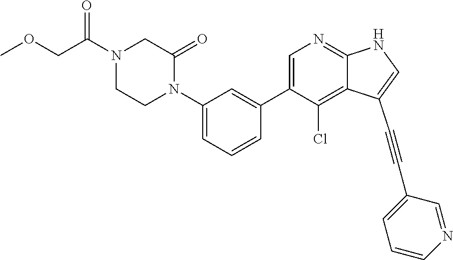 [SMILES: COCC(=O)N1CCN(c2cccc(-c3cnc4[nH]cc(C#Cc5cccnc5)c4c3Cl)c2)C(=O)C1] | 29 |
| 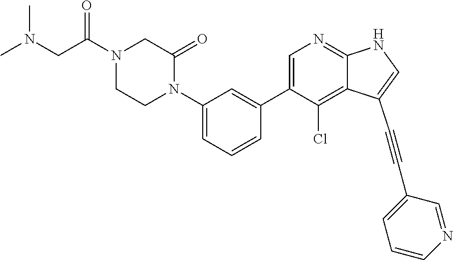 [SMILES: CN(C)CC(=O)N1CCN(c2cccc(-c3cnc4[nH]cc(C#Cc5cccnc5)c4c3Cl)c2)C(=O)C1] | 30 |
| 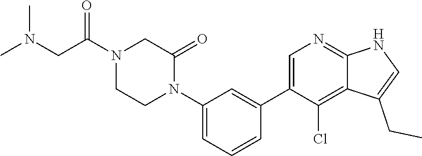 [SMILES: CCc1c[nH]c2ncc(-c3cccc(N4CCN(C(=O)CN(C)C)CC4=O)c3)c(Cl)c12] | 31 |
| 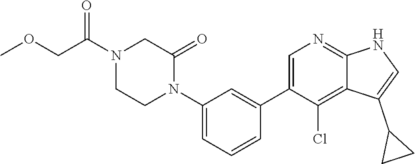 [SMILES: COCC(=O)N1CCN(c2cccc(-c3cnc4[nH]cc(C5CC5)c4c3Cl)c2)C(=O)C1] | 32 |
| 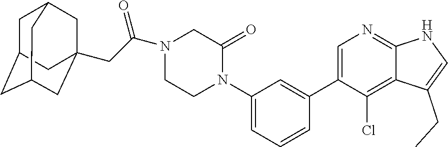 [SMILES: CCc1c[nH]c2ncc(-c3cccc(N4CCN(C(=O)CC56CC7CC(CC(C7)C5)C6)CC4=O)c3)c(Cl)c12] | 33 |
| 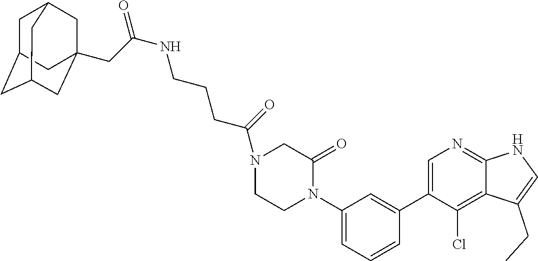 [SMILES: CCc1c[nH]c2ncc(-c3cccc(N4CCN(C(=O)CCCNC(=O)CC56CC7CC(CC(C7)C5)C6)CC4=O)c3)c(Cl)c12] | 34 |
| 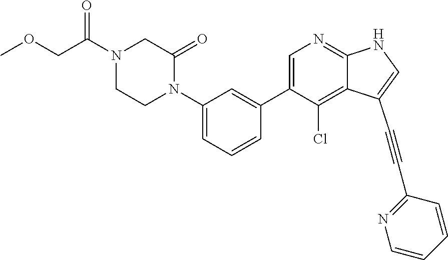 [SMILES: COCC(=O)N1CCN(c2cccc(-c3cnc4[nH]cc(C#Cc5ccccn5)c4c3Cl)c2)C(=O)C1] | 35 |
| 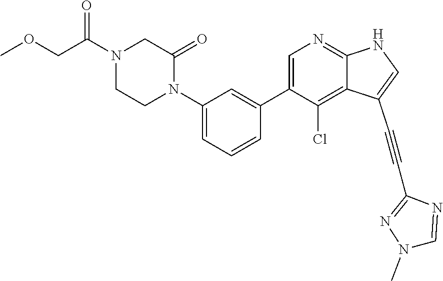 [SMILES: COCC(=O)N1CCN(c2cccc(-c3cnc4[nH]cc(C#Cc5ncn(C)n5)c4c3Cl)c2)C(=O)C1] | 36 |
| 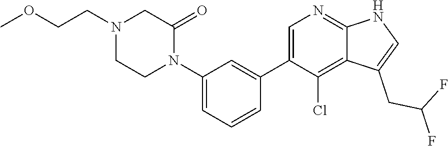 [SMILES: COCCN1CCN(c2cccc(-c3cnc4[nH]cc(CC(F)F)c4c3Cl)c2)C(=O)C1] | 37 |
| 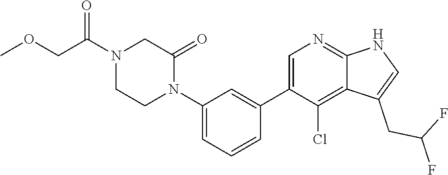 [SMILES: COCC(=O)N1CCN(c2cccc(-c3cnc4[nH]cc(CC(F)F)c4c3Cl)c2)C(=O)C1] | 38 |
| 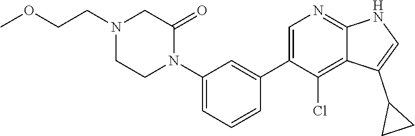 [SMILES: COCCN1CCN(c2cccc(-c3cnc4[nH]cc(C5CC5)c4c3Cl)c2)C(=O)C1] | 39 |
| 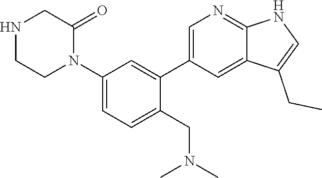 [SMILES: CCc1c[nH]c2ncc(-c3cc(N4CCNCC4=O)ccc3CN(C)C)cc12] | 40 |
| 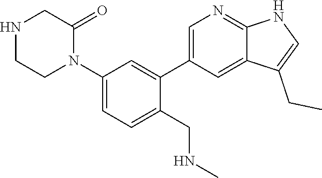 [SMILES: CCc1c[nH]c2ncc(-c3cc(N4CCNCC4=O)ccc3CNC)cc12] | 41 |
| 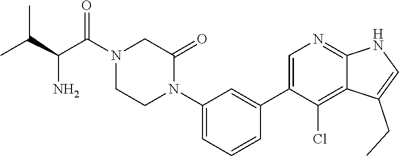 [SMILES: CCc1c[nH]c2ncc(-c3cccc(N4CCN(C(=O)[C@@H](N)C(C)C)CC4=O)c3)c(Cl)c12] | 42 |
| 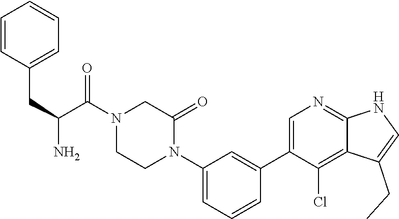 [SMILES: CCc1c[nH]c2ncc(-c3cccc(N4CCN(C(=O)[C@@H](N)Cc5ccccc5)CC4=O)c3)c(Cl)c12] | 43 |
| 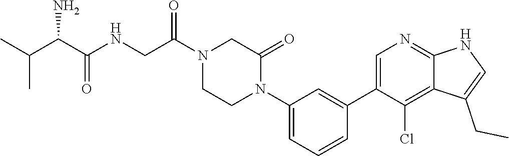 [SMILES: CCc1c[nH]c2ncc(-c3cccc(N4CCN(C(=O)CNC(=O)[C@@H](N)C(C)C)CC4=O)c3)c(Cl)c12] | 44 |
| 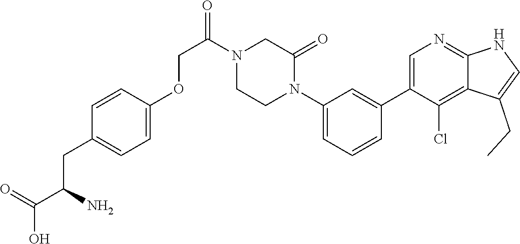 [SMILES: CCc1c[nH]c2ncc(-c3cccc(N4CCN(C(=O)COc5ccc(C[C@@H](N)C(=O)O)cc5)CC4=O)c3)c(Cl)c12] | 45 |
| 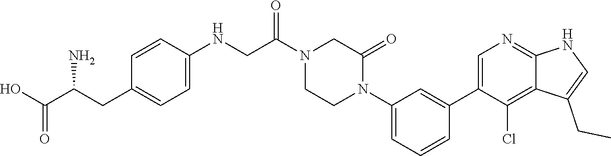 [SMILES: CCc1c[nH]c2ncc(-c3cccc(N4CCN(C(=O)CNc5ccc(C[C@@H](N)C(=O)O)cc5)CC4=O)c3)c(Cl)c12] | 46 |
| 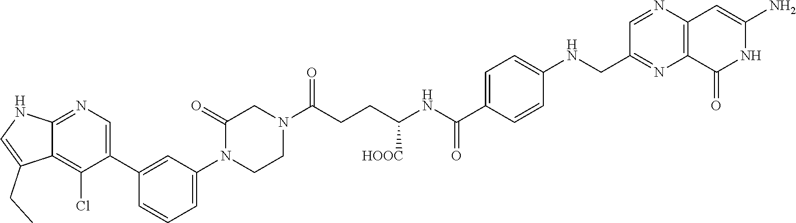 [SMILES: CCc1c[nH]c2ncc(-c3cccc(N4CCN(C(=O)CC[C@H](NC(=O)c5ccc(NCc6cnc7cc(N)[nH]c(=O)c7n6)cc5)C(=O)O)CC4=O)c3)c(Cl)c12] | 47 |
| 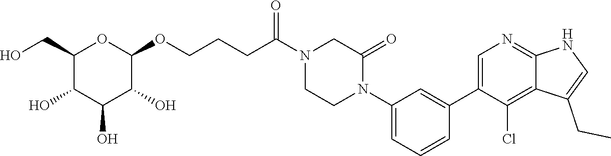 [SMILES: CCc1c[nH]c2ncc(-c3cccc(N4CCN(C(=O)CCCO[C@@H]5O[C@H](CO)[C@@H](O)[C@H](O)[C@H]5O)CC4=O)c3)c(Cl)c12] | 48 |
| 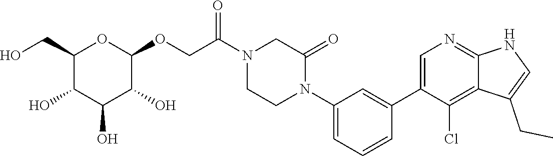 [SMILES: CCc1c[nH]c2ncc(-c3cccc(N4CCN(C(=O)CO[C@@H]5O[C@H](CO)[C@@H](O)[C@H](O)[C@H]5O)CC4=O)c3)c(Cl)c12] | 49 |
| 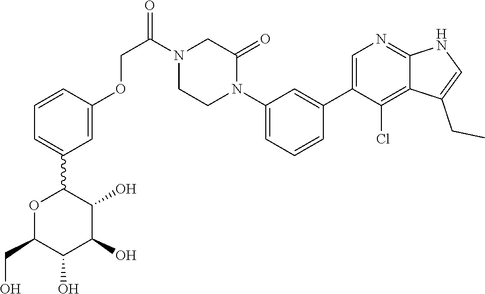 [SMILES: CCc1c[nH]c2ncc(-c3cccc(N4CCN(C(=O)COc5cccc(C6O[C@H](CO)[C@@H](O)[C@H](O)[C@H]6O)c5)CC4=O)c3)c(Cl)c12] | 50 |
| 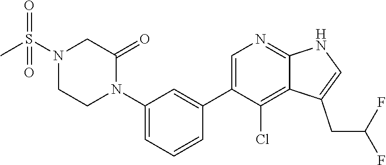 [SMILES: CS(=O)(=O)N1CCN(c2cccc(-c3cnc4[nH]cc(CC(F)F)c4c3Cl)c2)C(=O)C1] | 51 |
| 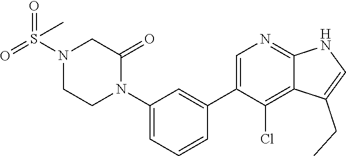 [SMILES: CCc1c[nH]c2ncc(-c3cccc(N4CCN(S(C)(=O)=O)CC4=O)c3)c(Cl)c12] | 52 |
| 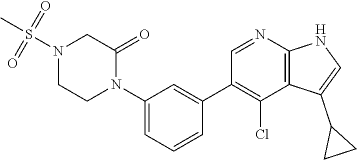 [SMILES: CS(=O)(=O)N1CCN(c2cccc(-c3cnc4[nH]cc(C5CC5)c4c3Cl)c2)C(=O)C1] | 53 |
| 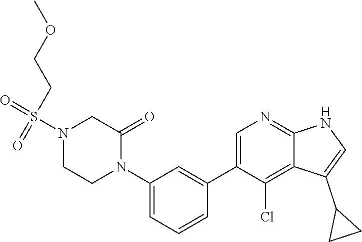 [SMILES: COCCS(=O)(=O)N1CCN(c2cccc(-c3cnc4[nH]cc(C5CC5)c4c3Cl)c2)C(=O)C1] | 54 |
| 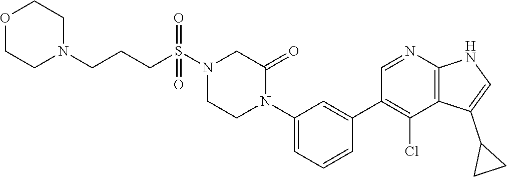 [SMILES: O=C1CN(S(=O)(=O)CCCN2CCOCC2)CCN1c1cccc(-c2cnc3[nH]cc(C4CC4)c3c2Cl)c1] | 55 |
| 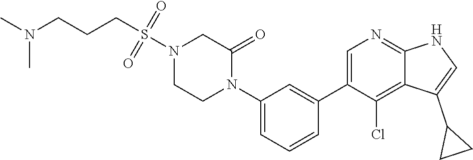 [SMILES: CN(C)CCCS(=O)(=O)N1CCN(c2cccc(-c3cnc4[nH]cc(C5CC5)c4c3Cl)c2)C(=O)C1] | 56 |
| 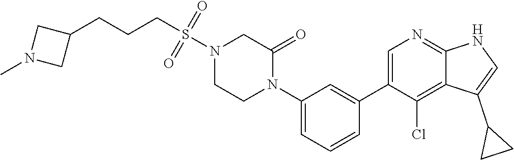 [SMILES: CN1CC(CCCS(=O)(=O)N2CCN(c3cccc(-c4cnc5[nH]cc(C6CC6)c5c4Cl)c3)C(=O)C2)C1] | 57 |
| 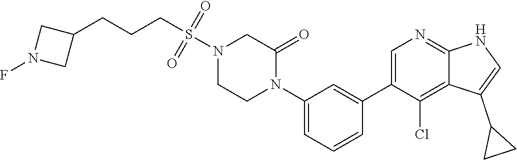 [SMILES: O=C1CN(S(=O)(=O)CCCC2CN(F)C2)CCN1c1cccc(-c2cnc3[nH]cc(C4CC4)c3c2Cl)c1] | 58 |
| 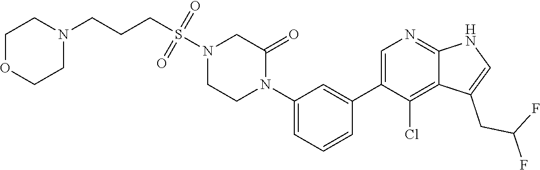 [SMILES: O=C1CN(S(=O)(=O)CCCN2CCOCC2)CCN1c1cccc(-c2cnc3[nH]cc(CC(F)F)c3c2Cl)c1] | 59 |
| 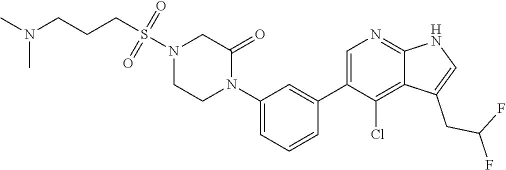 [SMILES: CN(C)CCCS(=O)(=O)N1CCN(c2cccc(-c3cnc4[nH]cc(CC(F)F)c4c3Cl)c2)C(=O)C1] | 60 |
| 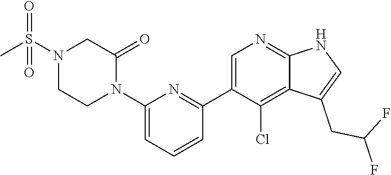 [SMILES: CS(=O)(=O)N1CCN(c2cccc(-c3cnc4[nH]cc(CC(F)F)c4c3Cl)n2)C(=O)C1] | 61 |
| 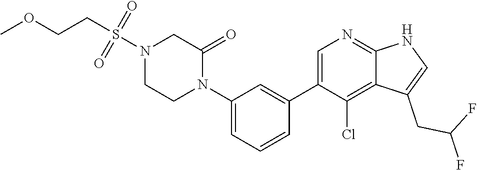 [SMILES: COCCS(=O)(=O)N1CCN(c2cccc(-c3cnc4[nH]cc(CC(F)F)c4c3Cl)c2)C(=O)C1] | 62 |

Another aspect of the present disclosure provides pharmaceutical compositions, comprising at least one compound selected from compounds of Formulae I, I′, IIA, IIA′, IIIA, IIIA′, IIIB, and IIIB′, Compounds 1 to 62, a tautomer thereof, a deuterated derivative of the compound or the tautomer, or a pharmaceutically acceptable salt of the foregoing, or a pharmaceutical composition comprising any of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of the present disclosure can be employed in combination therapies; that is, the pharmaceutical compositions disclosed herein can further comprise an additional active pharmaceutical agent. Alternatively, a pharmaceutical composition comprising a compound selected from compounds of Formulae I, I′, IIA, IIA′, IIIA, IIIA′, IIIB, and IIIB′, Compounds 1 to 62, a tautomer thereof, a deuterated derivative of the compound or the tautomer, or a pharmaceutically acceptable salt of the foregoing, or a pharmaceutical composition comprising any of the foregoing can be administered as a separate composition concurrently with, prior to, or subsequent to, a composition comprising an additional active pharmaceutical agent.

As discussed above, the pharmaceutical compositions disclosed herein further comprise a pharmaceutically acceptable carrier. The pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of the present disclosure, such as by producing any undesirable biological effect or otherwise interacting in a deleterious manner with any other component(s) of the pharmaceutical composition, its use is contemplated to be within the scope of the present disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### III. Methods of Treatment and Uses

In another aspect of the present disclosure, a compound, tautomer, deuterated derivative, or pharmaceutically acceptable salt as disclosed herein, including a compound of Formulae I, I′, IIA, IIA′, IIIA, IIIA′, IIIB, and IIIB′, any of Compounds 1 to 62, a tautomer thereof, a deuterated derivative of the compound or the tautomer, or a pharmaceutically acceptable salt of the foregoing, or the pharmaceutical composition thereof, is for use in treating a disease, a disorder, or a condition mediated by the inhibition of HPK1. In another aspect, disclosed herein is use of a compound, tautomer, deuterated derivative, or pharmaceutically acceptable salt as disclosed herein, including a compound of Formulae I, I′, IIA, IIA′, IIIA, IIIA′, IIIB, and IIIB′, any of Compounds 1 to 62, a tautomer thereof, a deuterated derivative of the compound or the tautomer, or a pharmaceutically acceptable salt of the foregoing, or the pharmaceutical composition thereof, for the manufacture of a medicament for treating a disease, a disorder, or a condition mediated by the inhibition of HPK1 and/or FLT3. In yet another aspect, disclosed herein is a method of treating a disease, a disorder, or a condition mediated by the inhibition of HPK1 and/or FLT3 in a subject, comprising administering a therapeutically effective amount of a compound, tautomer, deuterated derivative, or pharmaceutically acceptable salt as disclosed herein, including a compound of Formulae I, I′, IIA, IIA′, IIIA, IIIA′, IIIB, and IIIB′, any of Compounds 1 to 62, a tautomer thereof, a deuterated derivative of the compound or the tautomer, or a pharmaceutically acceptable salt of the foregoing, or the pharmaceutical composition thereof.

In some embodiments, the disease, the disorder, or the condition is chosen from an HPK1 and/or FLT3-related disease. In some embodiments, the disease, the disorder, or the condition is selected from cancer, a dysregulated immune response, or a disease involved in aberrant HPK1 and/or FLT3 expression, activity, and/or signaling. In some embodiments, the cancer is chosen from brain cancer, breast cancer, respiratory tract and/or lung cancer, a reproductive organ cancer, bone cancer, digestive tract cancer, urinary tract cancer, eye cancer, liver cancer, kidney cancer, skin cancer, head and neck cancer, anal cancer, nervous system cancer, thyroid cancer, parathyroid cancer, a lymphoma, a sarcoma, and a leukemia.

In some embodiments, the brain cancer is chosen from brain stem and hypothalamic glioma, cerebellar and cerebral astrocytoma, glioblastoma multiforme, medulloblastoma, ependymoma, neuroectodermal, and pineal tumor. In some embodiments, the liver cancer is chosen from hepatocellular carcinoma (liver cell carcinomas with or without fibrolamellar variant), cholangiocarcinoma (intrahepatic bile duct carcinoma) and mixed hepatocellular cholangiocarcinoma. In some embodiments, the respiratory tract and/or lung cancer is chosen from small cell lung cancer, non-small cell lung cancer, bronchial adenoma, and pleuropulmonary blastoma. In some embodiments, the digestive tract cancer is chosen from anal, colon, rectal, gallbladder, gastric, esophagus cancer, stomach, pancreas, salivary gland, small intestine, and colorectal cancer. In some embodiments, the kidney cancer is chosen from renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor. In some embodiments, the skin cancer is chosen from malignant melanoma, squamous cell carcinoma, Kaposi's sarcoma, Merkel cell skin cancer and non-melanoma skin cancer. In some embodiments, the head and neck cancer is chosen from squamous cell cancer of the head and neck, laryngeal, hypopharyngeal, nasopharyngeal, oropharyngeal cancer, nasal and paranasal cancers, salivary gland cancer, lip and oral cavity cancer, and squamous cell. In some embodiments, the reproductive organ cancer is chosen from prostate cancer, testicular cancer, endometrial cancer, cervical cancer, ovarian cancer, vaginal cancer, vulvar cancer, and uterus sarcoma. In some embodiments, the ovarian cancer is chosen from serous tumor, endometrioid tumor, mucinous cystadenocarcinoma, granulasa cell tumor, Sertoli-Leydig cell tumor, and arrhenoblastoma. In some embodiments, the cervical cancer is chosen from squamous cell carcinoma, adenocarcinoma, adenosquamous carcinoma, small cell carcinoma, neuroendocrine tumor, glassy cell carcinoma, and villogladular adenocarcinoma. In some embodiments, the bone cancer is chosen from osteogenic sarcoma, fibrosarcoma, malignant fibrous histiocytoma, chondrosarcoma, Ewing's sarcoma, malignant lymphoma, multiple myeloma, malignant giant cell tumor chordoma, osteochondroma, benign chondroma, chondroblastoma, chondromyxofibroma, osteoid osteoma, and giant cell tumor. In some embodiments, the breast cancer is chosen from triple negative breast cancer, invasive ductal carcinoma, invasive lobular carcinoma, ductal carcinoma in situ, and lobular carcinoma in situ. In some embodiments, the sarcoma is chosen from sarcoma of the soft tissue, chondrosarcoma, Ewing's sarcoma, angiosarcoma, fibrosarcoma, myxoma, rhabdomyoma, fibroma, lipoma, harmatoma, teratoma, osteosarcoma, malignant fibrous histiocytoma, liposarcoma, lymphosarcoma and rhabdomyosarcoma. In some embodiments, the eye cancer is chosen from intraocular melanoma and retinoblastoma. In some embodiments, the hematological cancer is chosen from lymphoma, leukemia such as acute lymphoblastic leukemia (ALL), acute myelogenous leukemia (AML), chronic lymphocytic leukemia (CLL), chronic myelogenous leukemia (CML), hairy cell leukemia, diffuse large B-cell lymphoma (DLBCL), acute promyelocytic leukemia (APL), chronic neutrophilic leukemia (CNL), acute undifferentiated leukemia (AUL), anaplastic large-cell lymphoma (ALCL), prolymphocytic leukemia (PML), juvenile myelomonocytic leukemia (JMML), adult T-cell ALL, AML, with trilineage myelodysplasia (AMLITMDS), mixed lineage leukemia (MLL), myelodysplastic syndromes (MDSs), myeloproliferative disorders (MPD), mantle cell lymphoma, non-Hodgkin lymphoma (NHL, including relapsed or refractory NHL), Hodgkin lymphoma, multiple myeloma and combinations of said cancers. In some embodiments, the nervous system cancer is chosen from a cancer of the skull, a cancer of the meninges, brain cancer, glioblastoma, spinal cord cancer, a neuroblastoma, and Lhermitte-Duclos disease.

In another aspect of the present disclosure, a compound, tautomer, deuterated derivative, or pharmaceutically acceptable salt as disclosed herein, including a compound of Formulae I, I′, IIA, IIA′, IIA, IIIA′, IIIB, and IIIB′, any of Compounds 1 to 62, a tautomer thereof, a deuterated derivative of the compound or the tautomer, or a pharmaceutically acceptable salt of the foregoing, or the pharmaceutical composition thereof, is for use in inhibiting HPK1 and/or FLT3 activity. In another aspect, disclosed herein is use of a compound, tautomer, deuterated derivative, or pharmaceutically acceptable salt as disclosed herein, including a compound of Formulae I, I′, IIA, IIA′, IIIA, IIIA′, IIIB, and IIIB′, any of Compounds 1 to 62, a tautomer thereof, a deuterated derivative of the compound or the tautomer, or a pharmaceutically acceptable salt of the foregoing, or the pharmaceutical composition thereof, for the manufacture of a medicament for inhibiting HPK1 and/or FLT3 activity. In yet another aspect, disclosed herein is a method of inhibiting HPK1 and/or FLT3 activity, comprising administering a therapeutically effective amount of a compound, tautomer, deuterated derivative, or pharmaceutically acceptable salt as disclosed herein to a subject, including a compound of Formulae I, I′, IIA, IIA′, IIIA, IIIA′, IIIB, and IIIB′, any of Compounds 1 to 62, a tautomer thereof, a deuterated derivative of the compound or the tautomer, or a pharmaceutically acceptable salt of the foregoing, or the pharmaceutical composition thereof. In yet another aspect, disclosed herein is a method of inhibiting HPK1 and/or FLT3 activity, comprising contacting said HPK1 and/or FLT3 a compound, tautomer, deuterated derivative, or pharmaceutically acceptable salt as disclosed herein to a subject, including a compound of Formulae I, I′, IIA, IIA′, IIIA, IIIA′, IIIB, and IIIB′, any of Compounds 1 to 62, a tautomer thereof, a deuterated derivative of the compound or the tautomer, or a pharmaceutically acceptable salt of the foregoing, or the pharmaceutical composition thereof.

A compound of Formulae I, I′, IIA, IIA′, IIIA, IIIA′, IIIB, and IIIB′, any of Compounds 1 to 62, a tautomer thereof, a deuterated derivative of the compound or the tautomer, or a pharmaceutically acceptable salt of the foregoing, or the pharmaceutical composition thereof may be administered once daily, twice daily, or three times daily, for example, for the treatment of a disease, a disorder, or a condition mediated by the inhibition of HPK1 and/or FLT3.

In some embodiments, 2 mg to 1500 mg or 5 mg to 1000 mg of a compound of Formulae I, I′, IIA, IIA′, IIIA, IIIA′, IIIB, and IIIB′, any of Compounds 1 to 62, a tautomer thereof, a deuterated derivative of the compound or the tautomer, or a pharmaceutically acceptable salt of the foregoing, or the pharmaceutical composition thereof are administered once daily, twice daily, or three times daily.

A compound of Formulae I, I′, IIA, IIA′, IIIA, IIIA′, IIIB, and IIIB′, any of Compounds 1 to 62, a tautomer thereof, a deuterated derivative of the compound or the tautomer, or a pharmaceutically acceptable salt of the foregoing, or the pharmaceutical composition thereof may be administered, for example, by oral, parenteral, sublingual, topical, rectal, nasal, buccal, vaginal, transdermal, patch, pump administration or via an implanted reservoir, and the pharmaceutical compositions would be formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time. Other forms of administration contemplated in the present disclosure are as described in International Patent Application Nos. WO 2013/075083, WO 2013/075084, WO 2013/078320, WO 2013/120104, WO 2014/124418, WO 2014/151142, and WO 2015/023915.

Useful dosages or a therapeutically effective amount of a compound or pharmaceutically acceptable salt thereof as disclosed herein can be determined by comparing their in vitro activity and in vivo activity in animal models. Methods for the extrapolation of effective dosages in mice and other animals, to humans are known to the art; for example, see U.S. Pat. No. 4,938,949.

One of ordinary skill in the art would recognize that, when an amount of compound is disclosed, the relevant amount of a pharmaceutically acceptable salt form of the compound is an amount equivalent to the concentration of the free base of the compound. The amounts of the compounds, pharmaceutically acceptable salts, solvates, and deuterated derivatives disclosed herein are based upon the free base form of the reference compound. For example, “1000 mg of at least one compound chosen from compounds of Formula I and pharmaceutically acceptable salts thereof” includes 1000 mg of compound of Formula I) and a concentration of a pharmaceutically acceptable salt of compounds of Formula I equivalent to 1000 mg of compounds of Formula I.

### Non-Limiting Exemplary Embodiments

1. A compound of Formula (I):

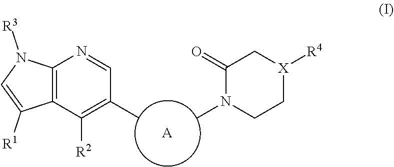

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) R² is chosen from hydrogen, halogen groups, OR^{x}, SR^{x}, NHR^{x}, N(R^{x})₂, CHR^{x}, and C(R^{x})₂;
(iii) R³ is chosen from hydrogen, linear, branched, and cyclic alkyl groups, heterocyclic groups, aryl groups, and heteroaryl groups;
(iv) R⁴ is chosen from hydrogen, linear, branched, and cyclic alkyl groups, heterocyclic groups, C(O)R^{y}, CO₂R^{y}, C(O)R^{w}OR^{y}, C(O)R^{w}N(R^{x}R^{y})₂, OC(O)R^{w}NR^{x}R^{y}, S(O)R^{y}, and SO₂R^{y};
(v) X is chosen from N and CR^{x};
(vi) each R^{x} and R^{y} is independently chosen from hydrogen, linear, branched, and cyclic alkyl groups, carbocyclic groups, heterocyclic groups, aryl groups, and heteroaryl groups;
(vii) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(viii) ring A is chosen from optionally substituted aryls, optionally substituted heteroaryls, optionally substituted cycloalkyls, and optionally substituted heterocycloalkyls;
wherein the linear, branched, and cyclic alkyl groups, linear, branched, and cyclic alkenyl groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups optionally substituted with 0, 1, or 2 C₁-C₆ linear, branched, and cyclic alkyl groups.
2. The compound, tautomer, deuterated derivative, or pharmaceutically acceptable salt of embodiment 1, wherein R¹ is chosen from linear, branched, and cyclic alkyl groups; R² is a halogen group; and R³ is chosen from hydrogen, linear, branched, and cyclic alkyl groups.
3. The compound, tautomer, deuterated derivative, or pharmaceutically acceptable salt of embodiment 1 or 2, wherein R¹ is chosen from C₁-C₆ linear, branched, and cyclic alkyl groups.
4. The compound, tautomer, deuterated derivative, or pharmaceutically acceptable salt of embodiment 3, wherein R¹ is chosen from methyl, ethyl, cyclopropyl, and cyclobutyl.
5. The compound, tautomer, deuterated derivative, or pharmaceutically acceptable salt of embodiment 1, wherein R¹ is a heterocyclic group.
6. The compound, tautomer, deuterated derivative, or pharmaceutically acceptable salt of embodiment 1, wherein R¹ is chosen from linear, branched, and cyclic alkynyl groups
7. The compound, tautomer, deuterated derivative, or pharmaceutically acceptable salt of embodiment 6, wherein the linear, branched, and cyclic alkynyl groups is substituted with at least one group chosen from C₁-C₆ linear, branched, and cyclic alkyl groups, C₁-C₆ linear, branched, and cyclic aminoalkyl groups, 3 to 6-membered heterocyclic groups, and 5 and 6-membered heteroaryl groups.
8. The compound, tautomer, deuterated derivative, or pharmaceutically acceptable salt of any of embodiments 1-7, wherein R² is a halogen group.
9. The compound, tautomer, deuterated derivative, or pharmaceutically acceptable salt of embodiment 8, wherein R² is fluoro.
10. The compound, tautomer, deuterated derivative, or pharmaceutically acceptable salt of embodiment 8, wherein R² is chloro.
11. The compound, tautomer, deuterated derivative, or pharmaceutically acceptable salt of any of embodiments 1-7, wherein R² is hydrogen.
12. The compound, tautomer, deuterated derivative, or pharmaceutically acceptable salt of any of embodiments 1-7, wherein R² is chosen from linear, branched, and cyclic alkyl groups.
13. The compound, tautomer, deuterated derivative, or pharmaceutically acceptable salt of embodiment 12, wherein R² is chosen from methyl, ethyl, n-propyl, iso-propyl, cyclopropyl, n-butyl, iso-butyl, sec-butyl, tert-butyl, and cyclobutyl.
14. The compound, tautomer, deuterated derivative, or pharmaceutically acceptable salt of any of embodiments 1-13, wherein R⁴ is hydrogen.
15. The compound, tautomer, deuterated derivative, or pharmaceutically acceptable salt of any of embodiments 1-13, wherein R⁴ is CO₂R^{y}, wherein R^{y} is chosen from linear, branched, and cyclic alkyl groups.
16. The compound, tautomer, deuterated derivative, or pharmaceutically acceptable salt of any of embodiments 1-13, wherein R⁴ is C(O)R^{y}, wherein R^{y} is chosen from linear, branched, and cyclic alkyl groups.
17. The compound, tautomer, deuterated derivative, or pharmaceutically acceptable salt of embodiment 15 or 16, wherein R^{y} is chosen from C₁-C₆ linear alkyl groups.
18. The compound, tautomer, deuterated derivative, or pharmaceutically acceptable salt of embodiment 17, wherein the C₁-C₆ linear alkyl groups is substituted with —N(C₁-C₆ linear, branched, and cyclic alkyl groups)₂.
19. The compound, tautomer, deuterated derivative, or pharmaceutically acceptable salt of embodiment 17, wherein the C₁-C₆ linear alkyl groups is substituted with C₁-C₆ linear, branched, and cyclic hydroxyalkyl groups.
20. The compound, tautomer, deuterated derivative, or pharmaceutically acceptable salt of embodiment 17, wherein the C₁-C₆ linear alkyl groups is substituted with C₁-C₆ linear, branched, and cyclic aminoalkyl groups,
21. The compound, tautomer, deuterated derivative, or pharmaceutically acceptable salt of embodiment 17, wherein the C₁-C₆ linear alkyl groups is substituted with C₁-C₆ linear, branched, and cyclic alkoxy groups.
22. The compound, tautomer, deuterated derivative, or pharmaceutically acceptable salt of any of embodiments 1-13, wherein R⁴ is SO₂R^{y}, wherein R^{y} is chosen from linear, branched, and cyclic alkyl groups.
23. The compound, tautomer, deuterated derivative, or pharmaceutically acceptable salt of embodiment 22, wherein R^{y} is chosen C₁-C₆ linear alkyl groups.
24. The compound, tautomer, deuterated derivative, or pharmaceutically acceptable salt of any of embodiments 1-23, wherein ring A is chosen from aryl groups.
25. The compound, tautomer, deuterated derivative, or pharmaceutically acceptable salt of embodiment 24, wherein ring A is phenyl.
26. The compound, tautomer, deuterated derivative, or pharmaceutically acceptable salt of any of embodiments 1-23, wherein ring A is chosen from aryl groups, wherein the aryl group is substituted with halogen groups.
27. The compound, tautomer, deuterated derivative, or pharmaceutically acceptable salt of any of embodiments 1-23, wherein ring A is chosen from aryl groups, wherein the aryl group is substituted with C₁-C₆ linear, branched, and cyclic alkyl groups.
28. The compound, tautomer, deuterated derivative, or pharmaceutically acceptable salt of any of embodiments 1-23, wherein ring A is chosen from heteroaryl groups.
29. The compound, tautomer, deuterated derivative, or pharmaceutically acceptable salt of embodiment 28, wherein ring A is chosen from heteroaryl groups, wherein the heteroaryl group is substituted with halogen groups.
30. The compound, tautomer, deuterated derivative, or pharmaceutically acceptable salt of embodiment 28, wherein ring A is chosen from heteroaryl groups, wherein the heteroaryl group is substituted with C₁-C₆ linear, branched, and cyclic alkyl groups.
31. The compound, tautomer, deuterated derivative, or pharmaceutically acceptable salt of embodiment 28, wherein ring A is chosen from 6-membered heteroaryl groups.
32. The compound, tautomer, deuterated derivative, or pharmaceutically acceptable salt of embodiment 31, wherein ring A is chosen from 6-membered heteroaryl groups, wherein the 6-membered heteroaryl groups is substituted with halogen groups.
33. The compound, tautomer, deuterated derivative, or pharmaceutically acceptable salt of embodiment 31, wherein ring A is chosen from 6-membered heteroaryl groups, wherein the 6-membered heteroaryl groups is substituted with C₁-C₆ linear, branched, and cyclic alkyl groups.
34. The compound, tautomer, deuterated derivative, or pharmaceutically acceptable salt of any of embodiments 31-33, wherein ring A is a pyridine ring.
35. The compound, tautomer, deuterated derivative, or pharmaceutically acceptable salt of any of embodiments 31-33, wherein ring A is a pyrimidine ring.
36. The compound, tautomer, deuterated derivative, or pharmaceutically acceptable salt of embodiment 28, wherein ring A is chosen from 5-membered heteroaryl groups.
37. The compound, tautomer, deuterated derivative, or pharmaceutically acceptable salt of embodiment 36, wherein ring A is chosen from 5-membered heteroaryl groups, wherein the 5-membered heteroaryl groups is substituted with halogen groups.
38. The compound, tautomer, deuterated derivative, or pharmaceutically acceptable salt of embodiment 36, wherein ring A is chosen from 5-membered heteroaryl groups, wherein the 5-membered heteroaryl groups is substituted with C₁-C₆ linear, branched, and cyclic alkyl groups.
39. The compound, tautomer, deuterated derivative, or pharmaceutically acceptable salt of any of embodiments 36-38, wherein ring A is a thiazole ring.
40. A compound of Formula (IIA):

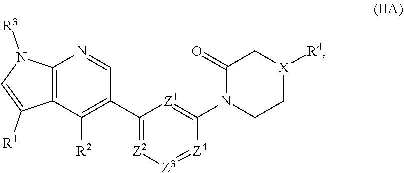

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) R² is chosen from hydrogen, halogen groups, OR^{x}, SR^{x}, NHR^{x}, N(R^{x})₂, CHR^{x}, and C(R^{x})₂;
(iii) R³ is chosen from hydrogen, linear, branched, and cyclic alkyl groups, heterocyclic groups, aryl groups, and heteroaryl groups;
(iv) R⁴ is chosen from hydrogen, linear, branched, and cyclic alkyl groups, heterocyclic groups, C(O)R^{y}, CO₂R^{y}, C(O)R^{w}OR^{y}, C(O)R^{w}N(R^{x}R^{y})₂, OC(O)R^{w}NR^{x}R^{y}, S(O)R^{y}, and SO₂R^{y};
(v) X is chosen from N and CR^{x};
(vi) each Z¹, Z², Z³, and Z⁴ is independently chosen from CR^{z} or N;
(vii) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(viii) each R^{x}, R^{y}, and R^{z} is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups optionally substituted with 0, 1, or 2 C₁-C₆ linear, branched, and cyclic alkyl groups.
41. The compound, tautomer, deuterated derivative, or pharmaceutically acceptable salt of embodiment 40, wherein R¹ is chosen from linear, branched, and cyclic alkyl groups; R² is a halogen group; and R³ is chosen from hydrogen, linear, branched, and cyclic alkyl groups.
42. The compound, tautomer, deuterated derivative, or pharmaceutically acceptable salt of embodiment 40 or 41, wherein R¹ is chosen from C₁-C₆ linear, branched, and cyclic alkyl groups.
43. The compound, tautomer, deuterated derivative, or pharmaceutically acceptable salt of embodiment 42, wherein R¹ is chosen from methyl, ethyl, cyclopropyl, and cyclobutyl.
44. The compound, tautomer, deuterated derivative, or pharmaceutically acceptable salt of embodiment 40, wherein R¹ is a heterocyclic group.
45. The compound, tautomer, deuterated derivative, or pharmaceutically acceptable salt of embodiment 42, wherein R¹ is chosen from linear, branched, and cyclic alkynyl groups
46. The compound, tautomer, deuterated derivative, or pharmaceutically acceptable salt of embodiment 45, wherein the linear, branched, and cyclic alkynyl groups is substituted with at least one group chosen from C₁-C₆ linear, branched, and cyclic alkyl groups, C₁-C₆ linear, branched, and cyclic aminoalkyl groups, 3 to 6-membered heterocyclic groups, and 5 and 6-membered heteroaryl groups.
47. The compound, tautomer, deuterated derivative, or pharmaceutically acceptable salt of any of embodiments 40-46, wherein R² is a halogen group.
48. The compound, tautomer, deuterated derivative, or pharmaceutically acceptable salt of embodiment 47, wherein R² is fluoro.
49. The compound, tautomer, deuterated derivative, or pharmaceutically acceptable salt of embodiment 49, wherein R² is chloro.
50. The compound, tautomer, deuterated derivative, or pharmaceutically acceptable salt of any of embodiments 40-46, wherein R² is hydrogen.
51. The compound, tautomer, deuterated derivative, or pharmaceutically acceptable salt of any of embodiments 40-46, wherein R² is chosen from linear, branched, and cyclic alkyl groups.
52. The compound, tautomer, deuterated derivative, or pharmaceutically acceptable salt of embodiment 51, wherein R² is chosen from methyl, ethyl, n-propyl, iso-propyl, cyclopropyl, n-butyl, iso-butyl, sec-butyl, tert-butyl, and cyclobutyl.
53. The compound, tautomer, deuterated derivative, or pharmaceutically acceptable salt of any of embodiments 40-52, wherein R⁴ is hydrogen.
54. The compound, tautomer, deuterated derivative, or pharmaceutically acceptable salt of any of embodiments 40-52, wherein R⁴ is CO₂R^{y}, wherein R^{y} is chosen from linear, branched, and cyclic alkyl groups.
55. The compound, tautomer, deuterated derivative, or pharmaceutically acceptable salt of any of embodiments 1-13, wherein R⁴ is C(O)R^{y}, wherein R^{y} is chosen from linear, branched, and cyclic alkyl groups.
56. The compound, tautomer, deuterated derivative, or pharmaceutically acceptable salt of embodiment 54 or 55, wherein R^{y} is chosen C₁-C₆ linear alkyl groups.
57. The compound, tautomer, deuterated derivative, or pharmaceutically acceptable salt of embodiment 56, wherein the C₁-C₆ linear alkyl groups is substituted with —N(C₁-C₆ linear, branched, and cyclic alkyl groups)₂.
58. The compound, tautomer, deuterated derivative, or pharmaceutically acceptable salt of embodiment 56, wherein the C₁-C₆ linear alkyl groups is substituted with C₁-C₆ linear, branched, and cyclic hydroxyalkyl groups.
59. The compound, tautomer, deuterated derivative, or pharmaceutically acceptable salt of embodiment 56, wherein the C₁-C₆ linear alkyl groups is substituted with C₁-C₆ linear, branched, and cyclic aminoalkyl groups,
60. The compound, tautomer, deuterated derivative, or pharmaceutically acceptable salt of embodiment 56, wherein the C₁-C₆ linear alkyl groups is substituted with C₁-C₆ linear, branched, and cyclic alkoxy groups.
61. The compound, tautomer, deuterated derivative, or pharmaceutically acceptable salt of any of embodiments 40-52, wherein R⁴ is SO₂R^{y}, wherein R^{y} is chosen from linear, branched, and cyclic alkyl groups.
62. The compound, tautomer, deuterated derivative, or pharmaceutically acceptable salt of embodiment 61, wherein R^{y} is chosen C₁-C₆ linear alkyl groups.
63. The compound, tautomer, deuterated derivative, or pharmaceutically acceptable salt of any of embodiments 40-62, wherein Z¹ is N, Z² is CR^{z}, Z³ is CR^{z}, and Z⁴ is CR^{z}.
64. The compound, tautomer, deuterated derivative, or pharmaceutically acceptable salt of any of embodiments 40-62, wherein Z¹ is CR^{z}, Z² is N, Z³ is CR^{z}, and Z⁴ is CR^{z}.
65. The compound, tautomer, deuterated derivative, or pharmaceutically acceptable salt of any of embodiments 40-62, wherein^{z} is CR^{z}, Z² is CR^{z}, Z³ is N, and Z⁴ is CR^{z}.
66. The compound, tautomer, deuterated derivative, or pharmaceutically acceptable salt of any of embodiments 40-62, wherein Z¹ is CR^{z}, Z² is CR^{z}, Z³ is CR^{z}, and Z⁴ is N.
67. The compound, tautomer, deuterated derivative, or pharmaceutically acceptable salt of any of embodiments 40-62, wherein Z¹ is N, Z² is N, Z³ is CR^{z}, and Z⁴ is CR^{z}.
68. The compound, tautomer, deuterated derivative, or pharmaceutically acceptable salt of any of embodiments 40-62, wherein Z¹ is N, Z² is CR^{z}, Z³ is N, and Z⁴ is CR^{z}.
69. The compound, tautomer, deuterated derivative, or pharmaceutically acceptable salt of any of embodiments 40-62, wherein Z¹ is CR^{z}, Z² is CR^{z}, Z³ is CR^{z}, and Z⁴ is N.
70. The compound, tautomer, deuterated derivative, or pharmaceutically acceptable salt of any of embodiments 40-62, wherein Z¹ is N, Z² is CR^{z}, Z³ is CR^{z}, and Z⁴ is N.
71. A compound of Formula (IIIA):

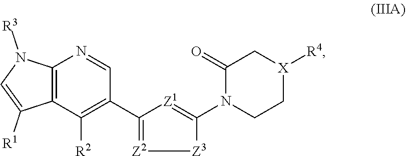

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) R² is chosen from hydrogen, halogen groups, OR^{x}, SR^{x}, NHR^{x}, N(R^{x})₂, CHR^{x}, and C(R^{x})₂;
(iii) R³ is chosen from hydrogen, linear, branched, and cyclic alkyl groups, heterocyclic groups, aryl groups, and heteroaryl groups;
(iv) R⁴ is chosen from hydrogen, linear, branched, and cyclic alkyl groups, heterocyclic groups, C(O)R^{y}, CO₂R^{y}, C(O)R^{w}OR^{y}, C(O)R^{w}N(R^{x}R^{y})₂, OC(O)R^{w}NR^{x}R^{y}, S(O)R^{y}, and SO₂R^{y};
(v) X is chosen from N and CR^{x};
(vi) each Z¹ and Z² is independently chosen from CR^{z} or N;
(vii) Z³ is chosen from O, S, and NR^{z}; and
(viii) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(ix) each R^{x}, R^{y}, and R^{z} is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
      5 and 6-membered heteroaryl groups optionally substituted with 0, 1, or 2 C₁-C₆ linear, branched, and cyclic alkyl groups.
72. The compound, tautomer, deuterated derivative, or pharmaceutically acceptable salt of embodiment 71, wherein R¹ is chosen from linear, branched, and cyclic alkyl groups; R² is a halogen group; and R³ is chosen from hydrogen, linear, branched, and cyclic alkyl groups.
73. The compound, tautomer, deuterated derivative, or pharmaceutically acceptable salt of embodiment 71 or 72, wherein R¹ is chosen from C₁-C₆ linear, branched, and cyclic alkyl groups.
74. The compound, tautomer, deuterated derivative, or pharmaceutically acceptable salt of embodiment 73, wherein R¹ is chosen from methyl, ethyl, cyclopropyl, and cyclobutyl.
75. The compound, tautomer, deuterated derivative, or pharmaceutically acceptable salt of embodiment 71, wherein R¹ is a heterocyclic group.
76. The compound, tautomer, deuterated derivative, or pharmaceutically acceptable salt of embodiment 71, wherein R¹ is chosen from linear, branched, and cyclic alkynyl groups
77. The compound, tautomer, deuterated derivative, or pharmaceutically acceptable salt of embodiment 76, wherein the linear, branched, and cyclic alkynyl groups is substituted with at least one group chosen from C₁-C₆ linear, branched, and cyclic alkyl groups, C₁-C₆ linear, branched, and cyclic aminoalkyl groups, 3 to 6-membered heterocyclic groups, and 5 and 6-membered heteroaryl groups.
78. The compound, tautomer, deuterated derivative, or pharmaceutically acceptable salt of any of embodiments 71-77, wherein R² is a halogen group.
79. The compound, tautomer, deuterated derivative, or pharmaceutically acceptable salt of embodiment 78, wherein R² is fluoro.
80. The compound, tautomer, deuterated derivative, or pharmaceutically acceptable salt of embodiment 78, wherein R² is chloro.
81. The compound, tautomer, deuterated derivative, or pharmaceutically acceptable salt of any of embodiments 71-77, wherein R² is hydrogen.
82. The compound, tautomer, deuterated derivative, or pharmaceutically acceptable salt of any of embodiments 71-77, wherein R² is chosen from linear, branched, and cyclic alkyl groups.
83. The compound, tautomer, deuterated derivative, or pharmaceutically acceptable salt of embodiment 82, wherein R² is chosen from methyl, ethyl, n-propyl, iso-propyl, cyclopropyl, n-butyl, iso-butyl, sec-butyl, tert-butyl, and cyclobutyl.
84. The compound, tautomer, deuterated derivative, or pharmaceutically acceptable salt of any of embodiments 71-83, wherein R⁴ is hydrogen.
85. The compound, tautomer, deuterated derivative, or pharmaceutically acceptable salt of any of embodiments 71-83, wherein R⁴ is CO₂R^{y}, wherein R^{y} is chosen from linear, branched, and cyclic alkyl groups.
86. The compound, tautomer, deuterated derivative, or pharmaceutically acceptable salt of any of embodiments 71-83, wherein R⁴ is C(O)R^{y}, wherein R^{y} is chosen from linear, branched, and cyclic alkyl groups.
87. The compound, tautomer, deuterated derivative, or pharmaceutically acceptable salt of embodiment 85 or 86, wherein R^{y} is chosen C₁-C₆ linear alkyl groups.
88. The compound, tautomer, deuterated derivative, or pharmaceutically acceptable salt of embodiment 87, wherein the C₁-C₆ linear alkyl groups is substituted with —N(C₁-C₆ linear, branched, and cyclic alkyl groups)₂.
89. The compound, tautomer, deuterated derivative, or pharmaceutically acceptable salt of embodiment 87, wherein the C₁-C₆ linear alkyl groups is substituted with C₁-C₆ linear, branched, and cyclic hydroxyalkyl groups.
90. The compound, tautomer, deuterated derivative, or pharmaceutically acceptable salt of embodiment 87, wherein the C₁-C₆ linear alkyl groups is substituted with C₁-C₆ linear, branched, and cyclic aminoalkyl groups,
91. The compound, tautomer, deuterated derivative, or pharmaceutically acceptable salt of embodiment 87, wherein the C₁-C₆ linear alkyl groups is substituted with C₁-C₆ linear, branched, and cyclic alkoxy groups.
92. The compound, tautomer, deuterated derivative, or pharmaceutically acceptable salt of any of embodiments 71-83, wherein R⁴ is SO₂R^{y}, wherein R^{y} is chosen from linear, branched, and cyclic alkyl groups.
93. The compound, tautomer, deuterated derivative, or pharmaceutically acceptable salt of embodiment 92, wherein R^{y} is chosen C₁-C₆ linear alkyl groups.
94. The compound, tautomer, deuterated derivative, or pharmaceutically acceptable salt of any of embodiments 71-93, wherein Z¹ is CR^{z}, Z² is CR^{z}, and Z³ is O.
95. The compound, tautomer, deuterated derivative, or pharmaceutically acceptable salt of any of embodiments 71-93, wherein Z¹ is CR^{z}, Z² is CR^{z}, and Z³ is S.
96. The compound, tautomer, deuterated derivative, or pharmaceutically acceptable salt of any of embodiments 71-93, wherein Z¹ is N, Z² is CR^{z}, and Z³ is O.
97. The compound, tautomer, deuterated derivative, or pharmaceutically acceptable salt of any of embodiments 71-93, wherein Z¹ is N, Z² is CR^{z}, and Z³ is S.
98. The compound, tautomer, deuterated derivative, or pharmaceutically acceptable salt of any of embodiments 71-93, wherein Z¹ is CR^{z}, Z² is N, and Z³ is O.
99. The compound, tautomer, deuterated derivative, or pharmaceutically acceptable salt of any of embodiments 71-93, wherein Z¹ is CR^{z}, Z² is N, and Z³ is S.
100. A compound of Formula (IIIB):

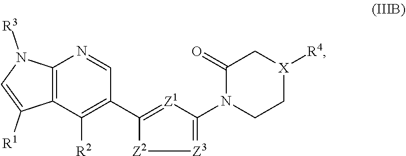

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) R² is chosen from hydrogen, halogen groups, OR^{x}, SR^{x}, NHR^{x}, N(R^{x})₂, CHR^{x}, and C(R^{x})₂;
(iii) R³ is chosen from hydrogen, linear, branched, and cyclic alkyl groups, heterocyclic groups, aryl groups, and heteroaryl groups;
(iv) R⁴ is chosen from hydrogen, linear, branched, and cyclic alkyl groups, heterocyclic groups, C(O)R^{y}, CO₂R^{y}, C(O)R^{w}OR^{y}, C(O)R^{w}N(R^{x}R^{y})₂, OC(O)R^{w}NR^{x}R^{y}, S(O)R^{y}, and SO₂R^{y};
(v) X is chosen from N and CR^{x};
(vi) each Z¹ and Z³ is independently chosen from CR^{z} or N;
(vii) Z² is chosen from O, S, and NR^{z};
(viii) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(ix) each R^{x}, R^{y}, and R^{z} is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups optionally substituted with 0, 1, or 2 C₁-C₆ linear, branched, and cyclic alkyl groups.
101. The compound, tautomer, deuterated derivative, or pharmaceutically acceptable salt of embodiment 100, wherein R¹ is chosen from linear, branched, and cyclic alkyl groups; R² is a halogen group; and R³ is chosen from hydrogen, linear, branched, and cyclic alkyl groups.
102. The compound, tautomer, deuterated derivative, or pharmaceutically acceptable salt of embodiment 100 or 101, wherein R¹ is chosen from C₁-C₆ linear, branched, and cyclic alkyl groups.
103. The compound, tautomer, deuterated derivative, or pharmaceutically acceptable salt of embodiment 102, wherein R¹ is chosen from methyl, ethyl, cyclopropyl, and cyclobutyl.
104. The compound, tautomer, deuterated derivative, or pharmaceutically acceptable salt of embodiment 100, wherein R¹ is a heterocyclic group.
105. The compound, tautomer, deuterated derivative, or pharmaceutically acceptable salt of embodiment 100, wherein R¹ is chosen from linear, branched, and cyclic alkynyl groups
106. The compound, tautomer, deuterated derivative, or pharmaceutically acceptable salt of embodiment 105, wherein the linear, branched, and cyclic alkynyl groups is substituted with at least one group chosen from C₁-C₆ linear, branched, and cyclic alkyl groups, C₁-C₆ linear, branched, and cyclic aminoalkyl groups, 3 to 6-membered heterocyclic groups, and 5 and 6-membered heteroaryl groups.
107. The compound, tautomer, deuterated derivative, or pharmaceutically acceptable salt of any of embodiments 100-106, wherein R² is a halogen group.
108. The compound, tautomer, deuterated derivative, or pharmaceutically acceptable salt of embodiment 107, wherein R² is fluoro.
109. The compound, tautomer, deuterated derivative, or pharmaceutically acceptable salt of embodiment 107, wherein R² is chloro.
110. The compound, tautomer, deuterated derivative, or pharmaceutically acceptable salt of any of embodiments 100-106, wherein R² is hydrogen.
111. The compound, tautomer, deuterated derivative, or pharmaceutically acceptable salt of any of embodiments 100-106, wherein R² is chosen from linear, branched, and cyclic alkyl groups.
112. The compound, tautomer, deuterated derivative, or pharmaceutically acceptable salt of embodiment 111, wherein R² is chosen from methyl, ethyl, n-propyl, iso-propyl, cyclopropyl, n-butyl, iso-butyl, sec-butyl, tert-butyl, and cyclobutyl.
113. The compound, tautomer, deuterated derivative, or pharmaceutically acceptable salt of any of embodiments 100-112, wherein R⁴ is hydrogen.
114. The compound, tautomer, deuterated derivative, or pharmaceutically acceptable salt of any of embodiments 100-112, wherein R⁴ is CO₂R^{y}, wherein R^{y} is chosen from linear, branched, and cyclic alkyl groups.
115. The compound, tautomer, deuterated derivative, or pharmaceutically acceptable salt of any of embodiments 100-112, wherein R⁴ is C(O)R^{y}, wherein R^{y} is chosen from linear, branched, and cyclic alkyl groups.
116. The compound, tautomer, deuterated derivative, or pharmaceutically acceptable salt of embodiment 114 or 115, wherein R^{y} is chosen C₁-C₆ linear alkyl groups.
117. The compound, tautomer, deuterated derivative, or pharmaceutically acceptable salt of embodiment 116, wherein the C₁-C₆ linear alkyl groups is substituted with —N(C₁-C₆ linear, branched, and cyclic alkyl groups)₂.
118. The compound, tautomer, deuterated derivative, or pharmaceutically acceptable salt of embodiment 116, wherein the C₁-C₆ linear alkyl groups is substituted with C₁-C₆ linear, branched, and cyclic hydroxyalkyl groups.
119. The compound, tautomer, deuterated derivative, or pharmaceutically acceptable salt of embodiment 116, wherein the C₁-C₆ linear alkyl groups is substituted with C₁-C₆ linear, branched, and cyclic aminoalkyl groups,
120. The compound, tautomer, deuterated derivative, or pharmaceutically acceptable salt of embodiment 116, wherein the C₁-C₆ linear alkyl groups is substituted with C₁-C₆ linear, branched, and cyclic alkoxy groups.
121. The compound, tautomer, deuterated derivative, or pharmaceutically acceptable salt of any of embodiments 100-112, wherein R⁴ is SO₂R^{y}, wherein R^{y} is chosen from linear, branched, and cyclic alkyl groups.
122. The compound, tautomer, deuterated derivative, or pharmaceutically acceptable salt of embodiment 121, wherein R^{y} is chosen C₁-C₆ linear alkyl groups.
123. The compound, tautomer, deuterated derivative, or pharmaceutically acceptable salt of any of embodiments 71-93, wherein Z¹ is CR^{z}, Z² is O, and Z³ is CR^{z}.
124. The compound, tautomer, deuterated derivative, or pharmaceutically acceptable salt of any of embodiments 71-93, wherein Z¹ is CR^{z}, Z² is S, and Z³ is CR^{z}.
125. The compound, tautomer, deuterated derivative, or pharmaceutically acceptable salt of any of embodiments 71-93, wherein Z¹ is N, Z² is O, and Z³ is CR^{z}.
126. The compound, tautomer, deuterated derivative, or pharmaceutically acceptable salt of any of embodiments 71-93, wherein Z¹ is N, Z² is S, and Z³ is CR^{z}.
127. The compound, tautomer, deuterated derivative, or pharmaceutically acceptable salt of any of embodiments 71-93, wherein Z¹ is CR^{z}, Z² is O, and Z³ is N.
128. The compound, tautomer, deuterated derivative, or pharmaceutically acceptable salt of any of embodiments 71-93, wherein Z¹ is CR^{z}, Z² is S, and Z³ is N.
129. A compound chosen from the compounds of Table 1, a tautomer thereof, a deuterated derivative of the compound or the tautomer, or a pharmaceutically acceptable salt of the foregoing.
130. A pharmaceutical composition comprising a compound, tautomer, deuterated derivative, or pharmaceutically acceptable salt according to any one of embodiments 1-129 and at least one pharmaceutically acceptable carrier.
131. A method for treating or alleviating a disease, a disorder or a condition mediated by the inhibition of hematopoietic progenitor kinase 1 (HPK1) and/or (Human Fms-like tyrosine kinase 3 (FLT3), comprising administering to a subject in need thereof a therapeutically effective amount of a compound, tautomer, deuterated derivative, or pharmaceutically acceptable salt according to any one of the embodiments 1-129 or the pharmaceutical composition according to embodiment 130.
132. A method for decreasing HPK1 and/or FLT3 activity in a disease, a disorder or a condition, comprising administering to a subject in need thereof a therapeutically effective amount of a compound, tautomer, deuterated derivative, or pharmaceutically acceptable salt according to any one of the embodiments 1-129 or the pharmaceutical composition according to embodiment 130.
133. The method of embodiment 132, wherein the disease, the disorder, or the condition is chosen from an HPK1 and/or FLT3-related disease.
134. The method of embodiment 132, wherein the HPK1 and/or FLT3 related disease is chosen from cancer, a dysregulated immune response, or a disease involved in aberrant HPK1 and/or FLT3 expression, activity, and/or signaling.
135. The method of embodiment 132, wherein the cancer is chosen from brain cancer, breast cancer, respiratory tract and/or lung cancer, a reproductive organ cancer, bone cancer, digestive tract cancer, urinary tract cancer, eye cancer, liver cancer, kidney cancer, skin cancer, head and neck cancer, anal cancer, nervous system cancer, thyroid cancer, parathyroid cancer, a lymphoma, a sarcoma, and a leukemia.
136. The method of embodiment 135, wherein the brain cancer is chosen from brain stem and hypothalamic glioma, cerebellar and cerebral astrocytoma, glioblastoma multiforme, medulloblastoma, ependymoma, neuroectodermal, and pineal tumor.
137. The method of embodiment 135, wherein the liver cancer is chosen from hepatocellular carcinoma (liver cell carcinomas with or without fibrolamellar variant), cholangiocarcinoma (intrahepatic bile duct carcinoma) and mixed hepatocellular cholangiocarcinoma.
138. The method of embodiment 135, wherein the respiratory tract and/or lung cancer is chosen from small cell lung cancer, non-small cell lung cancer, bronchial adenoma, and pleuropulmonary blastoma.
139. The method of embodiment 135, wherein the digestive tract cancer is chosen from anal, colon, rectal, gallbladder, gastric, esophagus cancer, stomach, pancreas, salivary gland, small-intestine, and colorectal cancer.
140. The method of embodiment 135, wherein the kidney cancer is chosen from renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor.
141. The method of embodiment 135, wherein the skin cancer is chosen from malignant melanoma, squamous cell carcinoma, Kaposi's sarcoma, Merkel cell skin cancer and non-melanoma skin cancer. The method of embodiment 127, wherein the head and neck cancer is chosen from squamous cell cancer of the head and neck, laryngeal, hypopharyngeal, nasopharyngeal, oropharyngeal cancer, nasal and paranasal cancers, salivary gland cancer, lip and oral cavity cancer, and squamous cell.
142. The method of embodiment 135, wherein the reproductive organ cancer is chosen from prostate cancer, testicular cancer, endometrial cancer, cervical cancer, ovarian cancer, vaginal cancer, vulvar cancer, and uterus sarcoma.
143. The method of embodiment 135, wherein the ovarian cancer is chosen from serous tumor, endometrioid tumor, mucinous cystadenocarcinoma, granulasa cell tumor, Sertoli-Leydig cell tumor, and arrhenoblastoma.
144. The method of embodiment 135, wherein the cervical cancer is chosen from squamous cell carcinoma, adenocarcinoma, adenosquamous carcinoma, small cell carcinoma, neuroendocrine tumor, glassy cell carcinoma, and villogladular adenocarcinoma.
145. The method of embodiment 135, wherein the bone cancer is chosen from osteogenic sarcoma, fibrosarcoma, malignant fibrous histiocytoma, chondrosarcoma, Ewing's sarcoma, malignant lymphoma, multiple myeloma, malignant giant cell tumor chordoma, osteochondroma, benign chondroma, chondroblastoma, chondromyxofibroma, osteoid osteoma, and giant cell tumor.
146. The method of embodiment 135, wherein the breast cancer is chosen from triple negative breast cancer, invasive ductal carcinoma, invasive lobular carcinoma, ductal carcinoma in situ, and lobular carcinoma in situ.
147. The method of embodiment 135, wherein the sarcoma is chosen from sarcoma of the soft tissue, chondrosarcoma, Ewing's sarcoma, angiosarcoma, fibrosarcoma, myxoma, rhabdomyoma, fibroma, lipoma, harmatoma, teratoma, osteosarcoma, malignant fibrous histiocytoma, liposarcoma, lymphosarcoma and rhabdomyosarcoma.
148. The method of embodiment 135, wherein the eye cancer is chosen from intraocular melanoma and retinoblastoma.
149. The method of embodiment 135, wherein the hematological cancer is chosen from lymphoma, leukemia such as acute lymphoblastic leukemia (ALL), acute myelogenous leukemia (AML), chronic lymphocytic leukemia (CLL), chronic myelogenous leukemia (CHL), hairy cell leukemia, diffuse large B-cell lymphoma (DLBCL), acute promyelocytic leukemia (APL), chronic neutrophilic leukemia (CNL), acute undifferentiated leukemia (AUL), anaplastic large-cell lymphoma (ALCL), prolymphocytic leukemia (PML), juvenile myelomonocytic leukemia (JMML), adult T-cell ALL, AML, with trilineage myelodysplasia (AMLITMDS), mixed lineage leukemia (MLL), myelodysplastic syndromes (MDSs), myeloproliferative disorders (MPD), mantle cell lymphoma, non-Hodgkin lymphoma (NHL, including relapsed or refractory NHL), Hodgkin lymphoma, multiple myeloma and combinations of said cancers.
150. The method of embodiment 135, wherein the nervous system cancer is chosen from a cancer of the skull, a cancer of the meninges, brain cancer, glioblastoma, spinal cord cancer, a neuroblastoma, and Lhermitte-Duclos disease.
151. The method of embodiment 135, further comprising the administration to the subject an existing standard treatment or an FDA-approved therapy.

### EXAMPLES

### Synthesis of Compounds

To fully understand the present disclosure, the following examples are disclosed. It should be understood that these examples are for illustrative purposes only and are not to be construed as limiting the present disclosure in any manner.

All the specific and generic compounds, and the intermediates disclosed for making those compounds, are considered to be part of the present disclosure.

The compounds of the present disclosure may be made according to standard chemical practices or as disclosed herein. Throughout the following synthetic schemes and in the descriptions for preparing compounds of Formulae I, I′, IIA, IIa′, IA, IIA′, IIIB, and IIIB′, Compounds 1 to 62, pharmaceutically acceptable salts of any of those compounds, solvates of any of the foregoing, and deuterated derivatives of any of the foregoing, the following abbreviations are used:

### Abbreviations

ACN=acetonitrile
Boc₂O=di-tert-butyl dicarbonate
DCE=1,2-dichloroethane
DCM=dichloromethane
DIEA=N,N-Diisopropylethylamine or N-ethyl-N-isopropyl-propan-2-amine
DMAP=dimethylamino pyridine
DME=dimethoxyethane
DMF=dimethylformamide
DMSO=dimethyl sulfoxide
EtOAc/EA=Ethyl Acetate
EtOH=ethanol
HOAc=acetic acid
KOAc=potassium acetate
MeOH=methanol
NaOAc=sodium acetate
NBS=N-bromosuccinimide
NIS=N-iodosuccinimide
NMP=N-methyl-2-pyrrolidone
PE=petroleum ether
Pd(dppf)₂Cl₂=[1,1′-Bis(diphenylphosphino)ferrocene]dichloropalladium(II)
PTSA=p-Toluenesulfonic acid monohydrate
rt=room (ambient) temperature
T₃P=2,4,6-Tripropyl-1,3,5,2,4,6-trioxatriphosphorinane-2,4,6-trioxide
TEA=triethylamine
TFA=trifluoroacetic acid
THF=tetrahydrofuran
TLC=thin layer chromatography
TsCl=p-toluene sulfonyl chloride

### Example 1

### 1-(3-(4-chloro-3-ethyl-1H-pyrrolo[2,3-b]pyridin-5-yl)phenyl)piperazin-2-one

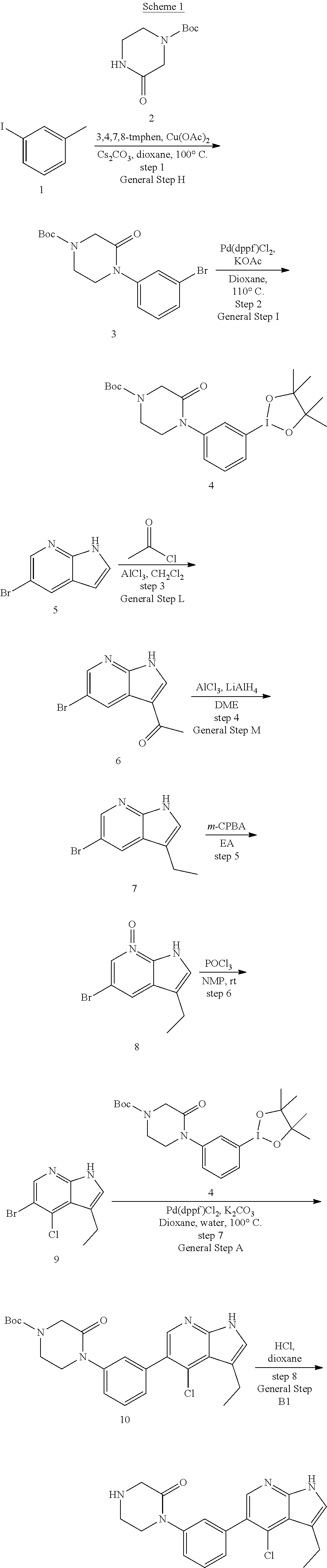

### Step 1. (General Step H) Preparation of tert-butyl 4-(3-bromophenyl)-3-oxopiperazine-1-carboxylate

To a solution of compound 1 (77.33 g, 0.273 mol), compound 2 (50 g, 0.248 mol), 3,4,7,8-tetramethyl-1,10-phenanthroline (17.62 g, 0.074 mol) in dioxane (1000 mL) under N₂ was added Cu(OAc)₂ (9 g, 0.05 mol) and Cs₂CO₃ (162 g, 0.5 mol). The reaction mixture was stirred at 100° C. for 16 hrs. The reaction was filtered and concentrated. The residue was slurry with EA/PE (1:10, 550 mL) for 2 hrs. The solid was filtered to afford compound 3 (70 g, 79% yield) as a yellow solid. Mass (m/z): 376.7[M+Na]⁺.

### Step 2. (General Step I) Preparation of tert-butyl 3-oxo-4-(3-(4,4,5,5-tetramethyl-1,3,2-dioxaborolan-2-yl)phenyl)piperazine-1-carboxylate

To a solution of compound 3 (107 g, 0.3 mol), 4,4,5,5-tetramethyl-2-(4,4,5,5-tetramethyl-1,3,2-dioxaborolan-2-yl)-1,3,2-dioxaborolane (91.5 g, 0.36 mol) and KOAc (88.45 g, 0.9 mol) in dioxane (1500 mL) under N₂ was added Pd(dppf)₂Cl₂ (14.9 g, 0.018 mol). The reaction mixture was stirred at 110° C. for 16 hrs. The reaction was filtered and concentrated. The reaction was added water (500 mL), extracted with EA (500 mL×3). The combined organic layers were washed with brine (1000 mL), dried over sodium sulfate, filtered and concentrated. The residue was purified by combi-flash with EA/PE (1:2) to afford compound 4 (92 g, 75.9% yield) as a white solid. Mass (m/z): 402.6[M+H]⁺.

### Step 3. (General Step L) Preparation of 1-(5-bromo-1H-pyrrolo[2,3-b]pyridin-3-yl)ethan-1-one

To a solution of 5-bromo-1H-pyrrolo[2,3-b]pyridine (50 g, 0.25 mol) in DCM (550 mL) was added AlCl₃ (101.27 g, 0.76 mol) and acetyl chloride (21.92 g, 0.28 mol) at 0° C. under N₂. The reaction mixture was stirred at rt under N₂ for 7 hrs. MeOH (300 mL) was added to the reaction mixture and the solvent was removed under reduced pressure. The reaction solution was adjusted to pH 6-7 with 3 N aqueous NaOH and extracted with EA (500 mL×3). The combined organic layer was washed with brine (300 mL×3), then dried over with anhydrous Na₂SO₄. After filtration, the solution was concentrated under vacuum, and the crude product was purified by Combiflash (PE/EtOAc=2:1) to give the product 1-(5-bromo-1H-pyrrolo[2,3-b]pyridin-3-yl)ethan-1-one as yellow solid (43.24 g, 71%). Mass (m/z): 241.0 [M+H]⁺.

### Step 4. (General Step M) Preparation of 5-bromo-3-ethyl-1H-pyrrolo[2,3-b]pyridine

To a solution of AlCl₃ (27.8 g, 0.20 mol) in DME (200 mL) were added LiAlH₄ (4.39 g, 0.1 mol) and 1-(5-bromo-1H-pyrrolo[2,3-b]pyridin-3-yl)ethan-1-one (10 g, 0.04 mol) at 0° C. The reaction mixture was stirred at rt under N₂ for 3 hrs. After the reaction completed, H₂O (500 mL) was added to the reaction mixture, and then extracted with EA (200 mL×3). The combined organic layer was washed with brine (100 mL×2), then dried over with anhydrous Na₂SO₄. The reaction mixture was filtered, the filtrate was concentrated under vacuum to afford compound product 5-bromo-3-ethyl-1H-pyrrolo[2,3-b]pyridine as yellow solid (11.5 g, 74%). Mass (m/z): 225.0 [M+H]⁺.

### Step 5. Preparation of 5-bromo-3-ethyl-1H-pyrrolo[2,3-b]pyridine 7-oxide

To a solution of 7 (25 g, 0.11 mol) in EA (100 mL) was added 3-Chloroperoxybenzoic acid (26.84 g, 0.155 mol). The reaction mixture was stirred at RT for 3 hrs. The solution was washed with sat. Na₂CO₃ (20 mL) and brine (20 mL), then dried over with anhydrous Na₂SO₄. The reaction mixture was filtered, the filtrate was concentrated to dryness to give the desired product 8 as a white solid (17.4 g, yield: 64.6%). Mass (m/z): 240.7 [M+H]⁺.

### Step 6. Preparation of 5-bromo-4-chloro-3-ethyl-1H-pyrrolo[2,3-b]pyridine

To a solution of 8 (17.3 g, 71.8 mmol) in NMP (15 mL) was added phosphoryl trichloride (55.05 g, 35.9 mmol) at 0° C. The reaction mixture was stirred at rt for 16 hrs. The mixture was quenched with water (50 mL), extracted with EA (30 mL×3), washed with sat brine, filtrated, concentrated, the residue was purified by flash column (PE/EA=5:1) to give the desired product 9 as a white solid (4.1 g, yield: 22%). Mass (m/z): 258.7 [M+H]⁺.

### Step 7. (General Step A) Preparation of tert-butyl 4-(3-(4-chloro-3-ethyl-1H-pyrrolo[2,3-b]pyridin-5-yl)phenyl)-3-oxopiperazine-1-carboxylate

To a mixture of compound 9 (4.1 g, 15.8 mmol), compound 4 (7.01 g, 17.38 mmol) and K₂CO₃ (6.55 g, 4.74 mmol) in dioxane/H₂O (10:1, 50 mL) under N₂ was added Pd(dppf)Cl₂ (1.16 g, 1.58 mmol). The reaction mixture was stirred at 90° C. for 4 hrs. The reaction was filtered and concentrated. The residue was purified by combi-flash with DCM/PE (1:2) to afford compound 10 (5.8 g, yield: 80%) as a yellow solid. Mass (m/z): 455.2[M+H]⁺.

### Step 8. (General Step B1) Preparation of 1-(3-(4-chloro-3-ethyl-1H-pyrrolo[2,3-b]pyridin-5-yl)phenyl) piperazin-2-one

To a mixture of compound 10 (5.8 g, 12.7 mmol) in DCM (20 mL) was added HCl in dioxane (20 mL). The reaction mixture was stirred at rt for 2 hrs. The reaction mixture was concentrated under reduced pressure. The residue was slurry with DCM (10 mL) for 1 h. The solid was filtered to afford Example 1 (4.1 g, yield: 91%) HCl salt as a yellow solid. Mass (m/z): 354.7[M+H]⁺. ¹H NMR (400 MHz, DMSO) δ 12.02 (s, 1H), 10.25 (s, 2H), 8.17 (s, 1H), 7.55 (d, J=7.8 Hz, 1H), 7.44 (dd, J=19.0, 10.0 Hz, 4H), 4.00 (t, J=5.0 Hz, 2H), 3.87 (s, 2H), 3.55 (s, 2H), 2.97-2.90 (m, 2H), 1.28 (t, J=7.4 Hz, 3H).

### Example 2

### Methyl 4-chloro-5-(3-(2-oxopiperazin-1-yl)phenyl)-1H-pyrrolo[2,3-b]pyridine-3-carboxylate

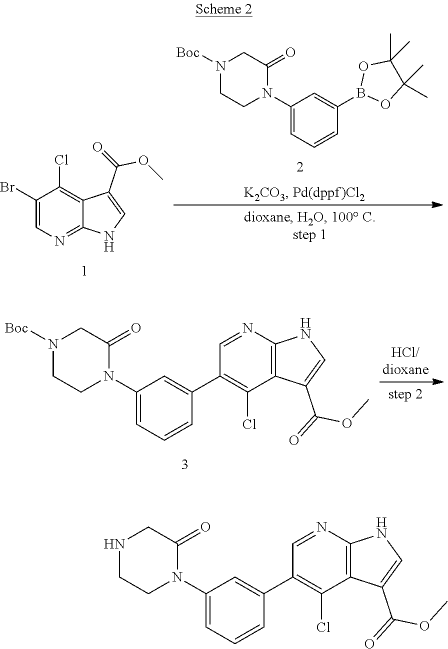 [SMILES: COC(=O)c1c[nH]c2ncc(Br)c(Cl)c12 ; CC(C)(C)OC(=O)N1CCN(c2cccc(B3OC(C)(C)C(C)(C)O3)c2)C(=O)C1 ; COC(=O)c1c[nH]c2ncc(-c3cccc(N4CCN(C(=O)OC(C)(C)C)CC4=O)c3)c(Cl)c12 ; COC(=O)c1c[nH]c2ncc(-c3cccc(N4CCNCC4=O)c3)c(Cl)c12]

### Step 1. Preparation of methyl 5-(3-(4-(tert-butoxycarbonyl)-2-oxopiperazin-1-yl)phenyl)-4-chloro-1H-pyrrolo[2,3-b]pyridine-3-carboxylate

Following General Step A, methyl 5-(3-(4-(tert-butoxycarbonyl)-2-oxopiperazin-1-yl)phenyl)-4-chloro-1H-pyrrolo[2,3-b]pyridine-3-carboxylate was prepared as a brown solid (140 mg, 41%). Mass (m/z): 484.8 [M+H]⁺.

### Step 2. Preparation of methyl 4-chloro-5-(3-(2-oxopiperazin-1-yl)phenyl)-1H-pyrrolo[2,3-b]pyridine-3-carboxylate

Following General Step B1, methyl 4-chloro-5-(3-(2-oxopiperazin-1-yl)phenyl)-1H-pyrrolo[2,3-b]pyridine-3-carboxylate was prepared as a brown solid (64 mg, 57%). Mass (m/z): 385.0 [M+H]⁺.

### Example 3

### 1-(3-(4-chloro-3-(3,3-dimethylbut-1-yn-1-yl)-1H-pyrrolo[2,3-b] pyridin-5-yl) phenyl) piperazin-2-one

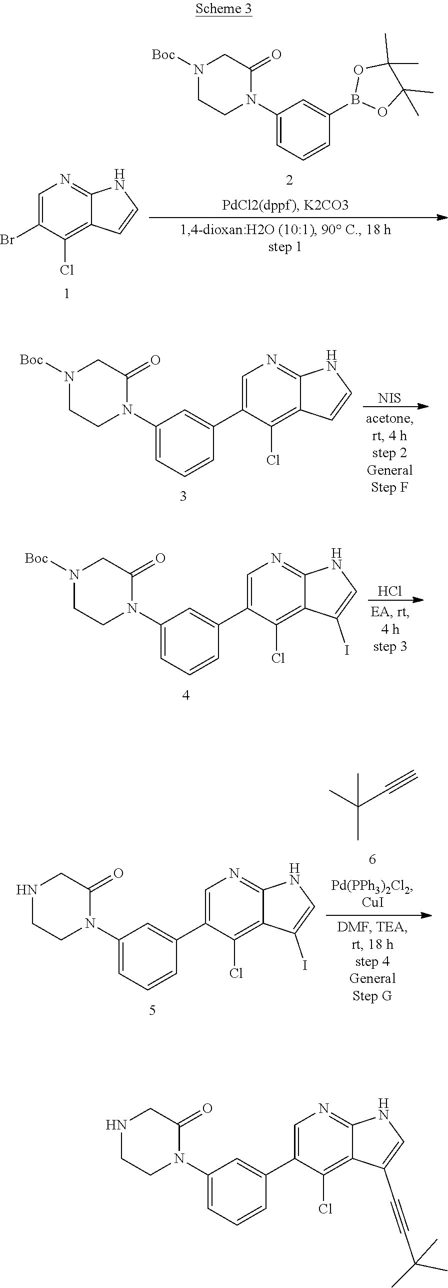

### Step 1. tert-butyl 4-(3-(4-chloro-1H-pyrrolo[2,3-b] pyridin-5-yl) phenyl)-3-oxopiperazine-1-carboxylate

Following General Step A, tert-butyl 4-(3-(4-chloro-1H-pyrrolo[2,3-b] pyridin-5-yl) phenyl)-3-oxopiperazine-1-carboxylate was prepared as a brown solid (15 g, 77% yield). MS: m/z=426.9. (M+1, ESI+).

### Step 2. (General Step F) tert-butyl 4-(3-(4-chloro-3-iodo-1H-pyrrolo[2,3-b] pyridin-5-yl) phenyl)-3-oxopiperazine-1-carboxylate

To a solution of tert-butyl[4-(3-{4-chloro-7H-pyrrolo[2,3-b]pyridin-3-yl}phenyl)-3-oxopiperazin-1-yl] formate (15 g, 35.1 mmol) in acetone (300 mL) was added NIS (8.69 g, 38.6 mmol). The resulting mixture was stirred at 25° C. for 4 hrs, and then concentrated. The residue was diluted with EA (200 mL), washed with water (100 mL×2), dried over Na₂SO₄, filtered and evaporated. The residue was purified by column chromatography (EA:PE=1:1) to give tert-butyl 4-(3-(4-chloro-3-iodo-1H-pyrrolo[2,3-b] pyridin-5-yl) phenyl)-3-oxopiperazine-1-carboxylate (8 g, 39% yield) as a yellow solid. MS: m/z=552.7. (M+1, ESI+).

### Step 3. 1-(3-(4-chloro-3-iodo-1H-pyrrolo[2,3-b]pyridin-5-yl)phenyl)piperazin-2-one

Following General Step B1, 1-(3-(4-chloro-3-iodo-1H-pyrrolo[2,3-b]pyridin-5-yl)phenyl)piperazin-2-one was prepared as a white solid (3 g, 70% yield). MS: m/z=452.6 (M+1, ESI+).

### Step 4. (General Step G) 1-(3-(4-chloro-3-(3,3-dimethylbut-1-yn-1-yl)-1H-pyrrolo[2,3-b] pyridin-5-yl) phenyl) piperazin-2-one

A mixture solution of 1-(3-{4-chloro-5-iodo-7H-pyrrolo[2,3-b]pyridin-3-yl}phenyl) piperazin-2-one (200 mg, 0.4418 mmol), 3,3-dimethylbut-1-yne (181.46 mg, 2.209 mmol), Bis(triphenylphosphine)palladium(II) chloride (31.01 mg, 0.0441 mmol), and Copper(I) iodide (16.83 mg, 0.0883 mmol) in TEA (2 mL) and DMF (2 mL) was stirred under nitrogen at RT for 18 hrs. Methyl tert-butyl ether was added. A white solid precipitated out and was filtered. The residue was purified by column chromatography (MeOH: DCM=1:10) to give crude product, which was further purified by Prep-HPLC (xbridge-c18 150×19 mm, 5 um, mobile term: ACN-H₂O (0.1% FA), gradient: 20-40) to give 1-(3-(4-chloro-3-(3,3-dimethylbut-1-yn-1-yl)-1H-pyrrolo[2,3-b] pyridin-5-yl) phenyl) piperazin-2-one (3 mg, 1.58% yield) as a yellow solid. MS: m/z=407 (M+1, ESI+).

### Example 4

### 1-(3-(4-chloro-3-cyclopropyl-1H-pyrrolo[2,3-b] pyridin-5-yl) phenyl) piperazin-2-one

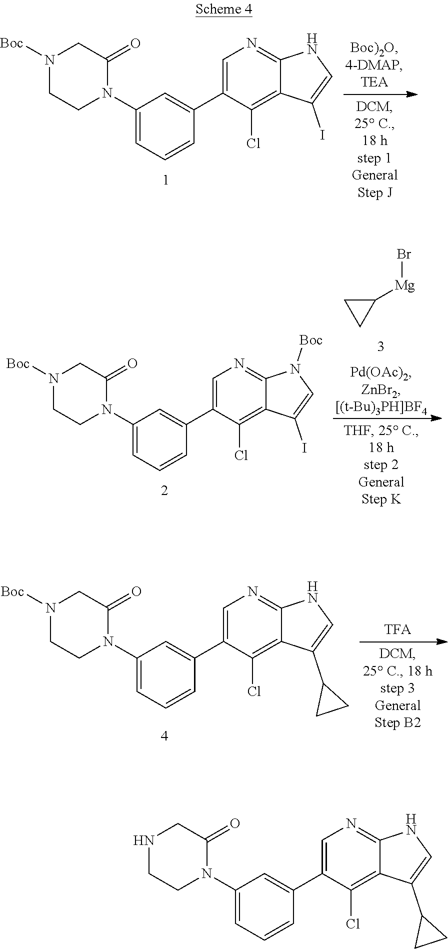

### Step 1. (General Step J) tert-butyl 5-(3-(4-(tert-butoxy carbonyl)-2-oxopiperazin-1-yl) phenyl)-4-chloro-3-iodo-1H-pyrrolo[2,3-b] pyridine-1-carboxylate

A mixture solution of tert-butyl 4-(3-(4-chloro-3-iodo-1H-pyrrolo[2,3-b]pyridin-5-yl)phenyl)-3-oxopiperazine-1-carboxylate (5 g, 9 mmol), 4-DMAP (0.11 g, 0.9 mmol), (Boc)₂O (2.95 g, 13.5 mmol) and TEA (01.82 g, 18 mmol) in DCM (50 mL) was stirred under nitrogen at RT overnight. The mixture was concentrated, and the residue was purified by column chromatography (eluting with EA:PE=1:1) to give tert-butyl 5-(3-(4-(tert-butoxy carbonyl)-2-oxopiperazin-1-yl) phenyl)-4-chloro-3-iodo-1H-pyrrolo[2,3-b] pyridine-1-carboxylate (2.5 g, 40%) as a yellow solid. MS: m/z=652.7 (M+1, ESI+).

### Step 2. (General Step K) tert-butyl 4-(3-(4-chloro-3-cyclopropyl-1H-pyrrolo[2,3-b]pyridin-5-yl) phenyl)-3-oxopiperazine-1-carboxylate

To a solution of tert-butyl 5-(3-(4-(tert-butoxy carbonyl)-2-oxopiperazin-1-yl) phenyl)-4-chloro-3-iodo-1H-pyrrolo[2,3-b] pyridine-1-carboxylate (500 mg, 0.763 mmol), Palladium diacetate (17.14 mg, 0.076 mmol), [(t-Bu)₃Ph]BF₄ (22.15 mg, 0.076 mmol), and Zinc bromide (171.92 mg, 0.763 mmol) in THF (5 mL) under nitrogen was added bromo(cyclopropyl)magnesium (1.5 mL, 1.53 mmol) dropwise at rt in 30 mins. The reaction mixture was stirred at rt for 18 hrs. Quenching with water, extraction with EA (20 mL*3), dried over Na₂SO₄ and filtered, evaporated, the residue was purified by column chromatography (EA:PE=1:1) to give tert-butyl 4-(3-(4-chloro-3-cyclopropyl-1H-pyrrolo[2,3-b] pyridin-5-yl) phenyl)-3-oxopiperazine-1-carboxylate (250 mg, 21%) as a black oil. MS: m/z=466.9 (M+1, ESI+).

### Step 3. (General Step B2) 1-(3-(4-chloro-3-cyclopropyl-1H-pyrrolo[2,3-b] pyridin-5-yl) phenyl) piperazin-2-one

A solution of tert-butyl[4-(3-{4-chloro-3-cyclopropyl-1H-pyrrolo[2,3-b] pyridin-5-yl} phenyl)-3-oxopiperazin-1-yl] formate (250 mg, 0.5342 mmol) in DCM (5 mL) and TFA (5 mL) was stirred at rt for 18 rs. After concentration, the residue was purified by Prep-HPLC (xbridge-c18 150×19 mm, 5 um, mobile term: ACN-H₂O (0.1% TA), gradient: 10-40) to give 1-(3-(4-chloro-3-cyclopropyl-1H-pyrrolo[2,3-b] pyridin-5-yl) phenyl) piperazin-2-one (14 mg, 6.42% yield) as a yellow solid. MS: m/z=367 (M+1, ESI+).

### Example 5

### 1-(3-(4-chloro-3-(cyclopropyl ethynyl)-1H-pyrrolo[2,3-b] pyridin-5-yl) phenyl) piperazin-2-one

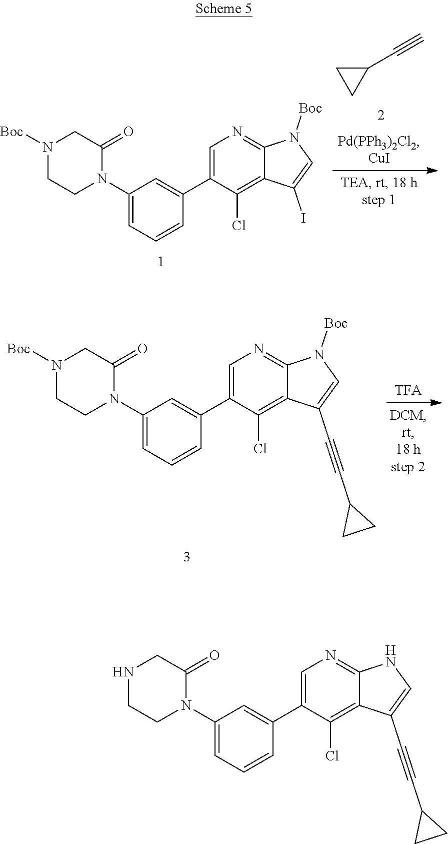

### Step 1. tert-butyl 5-(3-(4-(tert-butoxy carbonyl)-2-oxopiperazin-1-yl) phenyl)-4-chloro-3-(cyclopropyl ethynyl)-1H-pyrrolo[2,3-b] pyridine-1-carboxylate

Following General Step G, tert-butyl 5-(3-(4-(tert-butoxy carbonyl)-2-oxopiperazin-1-yl) phenyl)-4-chloro-3-(cyclopropyl ethynyl)-1H-pyrrolo[2,3-b] pyridine-1-carboxylate was prepared as a yellow solid (80 mg, 83%). MS: (m/z)=590.9 (M+1, ESI+).

### Step 2. 1-(3-(4-chloro-3-(cyclopropyl ethynyl)-1H-pyrrolo[2,3-b] pyridin-5-yl) phenyl) piperazin-2-one (Example 10)

Following General Step B2, 1-(3-(4-chloro-3-(cyclopropyl ethynyl)-1H-pyrrolo[2,3-b] pyridin-5-yl) phenyl) piperazin-2-one was prepared as a yellow solid (3 mg, 6%). MS: m/z=390.9 (M+1, ESI+).

### Example 6

### 1-(3-(4-chloro-3-(pyridin-3-ylethynyl)-1H-pyrrolo[2,3-b]pyridin-5-yl)phenyl)piperazin-2-one

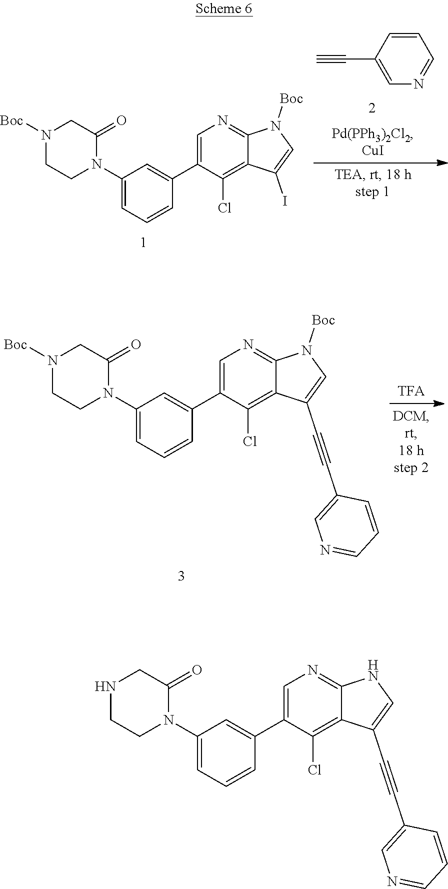

### Step 1. tert-butyl 5-(3-(4-(tert-butoxy carbonyl)-2-oxopiperazin-1-yl) phenyl)-4-chloro-3-(pyridin-3-ylethynyl)-1H-pyrrolo[2,3-b] pyridine-1-carboxylate

Following General Step G, tert-butyl 5-(3-(4-(tert-butoxy carbonyl)-2-oxopiperazin-1-yl) phenyl)-4-chloro-3-(pyridin-3-ylethynyl)-1H-pyrrolo[2,3-b] pyridine-1-carboxylate was prepared as a grey solid (300 mg, 74%). MS: (m/z)=627.8 (M+1, ESI+).

### Step 2. 1-(3-(4-chloro-3-(pyridin-3-ylethynyl)-1H-pyrrolo[2,3-b] pyridin-5-yl) phenyl) piperazin-2-one

Following General Step B2, 1-(3-(4-chloro-3-(pyridin-3-ylethynyl)-1H-pyrrolo[2,3-b] pyridin-5-yl) phenyl) piperazin-2-one (Example 11) was prepared as a gray solid (120 mg, 72%). MS (m/z)=427.8 (M+1, ESI+).

### Example 7

### 1-(3-(4-chloro-3-(pyridin-2-ylethynyl)-1H-pyrrolo[2,3-b] pyridin-5-yl) phenyl) piperazin-2-one

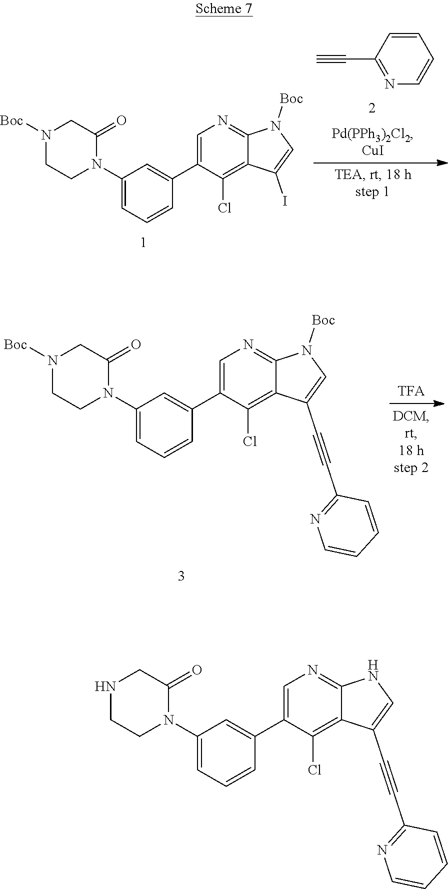

### Step 1. tert-butyl 5-(3-(4-(tert-butoxy carbonyl)-2-oxopiperazin-1-yl) phenyl)-4-chloro-3-(pyridin-2-ylethynyl)-1H-pyrrolo[2,3-b] pyridine-1-carboxylate

Following General Step G, tert-butyl 5-(3-(4-(tert-butoxy carbonyl)-2-oxopiperazin-1-yl) phenyl)-4-chloro-3-(pyridin-2-ylethynyl)-1H-pyrrolo[2,3-b] pyridine-1-carboxylate was prepared as a grey solid (150 mg, 74%). MS: m/z=627.8 (M+1, ESI+).

### Step 2. 1-(3-(4-chloro-3-(pyridin-2-ylethynyl)-1H-pyrrolo[2,3-b] pyridin-5-yl) phenyl) piperazin-2-one (Example 12

Following General Step B2, 1-(3-(4-chloro-3-(pyridin-2-ylethynyl)-1H-pyrrolo[2,3-b] pyridin-5-yl) phenyl) piperazin-2-one was prepared as a gray solid (80 mg, 71%). MS: m/z=427.8 (M+1, ESI+).

### Example 8

### 1-(3-(4-chloro-3-(3-(dimethylamino)prop-1-yn-1-yl)-1H-pyrrolo[2,3-b]pyridin-5-yl)phenyl)piperazin-2-one

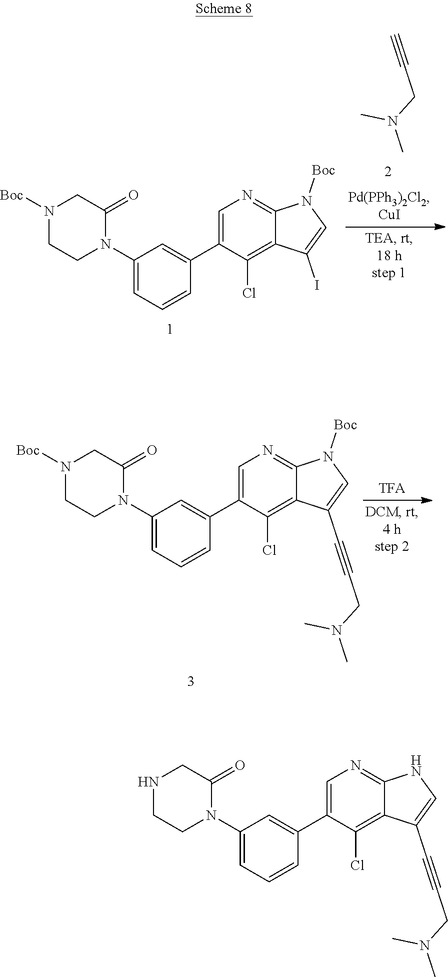

### Step 1. tert-butyl 5-(3-(4-(tert-butoxy carbonyl)-2-oxopiperazin-1-yl) phenyl)-4-chloro-3-(3-(dimethyl amino) prop-1-yn-1-yl)-1H-pyrrolo[2,3-b] pyridine-1-carboxylate

Following General Step G, tert-butyl 5-(3-(4-(tert-butoxy carbonyl)-2-oxopiperazin-1-yl) phenyl)-4-chloro-3-(3-(dimethyl amino) prop-1-yn-1-yl)-1H-pyrrolo[2,3-b] pyridine-1-carboxylate was prepared as a yellow solid (80 mg, 83%). MS: (m/z)=607.8 (M+1, ESI+).

### Step 2. 1-(3-(4-chloro-3-(3-(dimethyl amino) prop-1-yn-1-yl)-1H-pyrrolo[2,3-b]pyridin-5-yl) phenyl) piperazin-2-one (Example 13)

Following General Step B2, 1-(3-(4-chloro-3-(3-(dimethyl amino) prop-1-yn-1-yl)-1H-pyrrolo[2,3-b] pyridin-5-yl) phenyl) piperazin-2-one was prepared as a yellow solid (3 mg, 7%). MS: (m/z)=407.9 (M+1, ESI+).

### Example 9

### 1-(6-(4-chloro-3-ethyl-1H-pyrrolo[2,3-b]pyridin-5-yl)pyridin-2-yl)piperazin-2-one

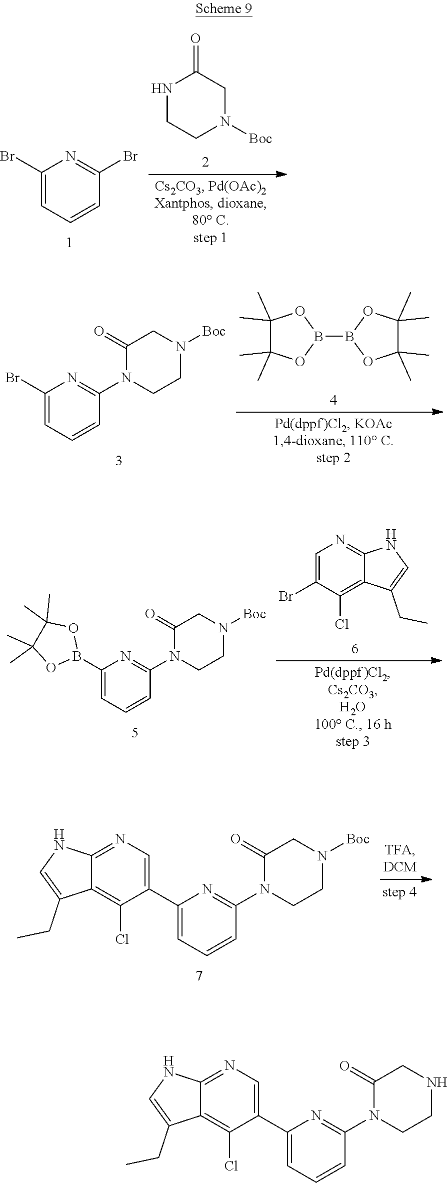

### Step 1. Preparation of [4-(6-bromopyridin-2-yl)-3-oxopiperazin-1-yl] tert-butyl formate

Following General Step H, [4-(6-bromopyridin-2-yl)-3-oxopiperazin-1-yl] tert-butyl formate was prepared as a brown solid (1.02 g, 26.8% yield). Mass (m/z): 355.8 [M+H]⁺.

### Step 2. Preparation of tert-butyl 3-oxo-4-(6-(4,4,5,5-tetramethyl-1,3,2-dioxaborolan-2-yl)pyridin-2-yl)piperazine-1-carboxylate

Following General Step I, tert-butyl 3-oxo-4-(6-(4,4,5,5-tetramethyl-1,3,2-dioxaborolan-2-yl)pyridin-2-yl)piperazine-1-carboxylate was prepared as a crude product, which was used in next step without further purification.

### Step 3. Preparation of tert-butyl 4-(6-(4-chloro-3-ethyl-1H-pyrrolo[2,3-b]pyridin-5-yl)pyridin-2-yl)-3-oxopiperazine-1-carboxylate

Following General Step A, tert-butyl 4-(6-(4-chloro-3-ethyl-1H-pyrrolo[2,3-b]pyridin-5-yl)pyridin-2-yl)-3-oxopiperazine-1-carboxylate was prepared as a brown solid (170 mg, 26% yield). Mass (m/z): 455.8 [M+H]⁺.

### Step 4. Preparation of 1-(6-(4-chloro-3-ethyl-1H-pyrrolo[2,3-b]pyridin-5-yl)pyridin-2-yl)piperazin-2-one

Following General Step B2, 1-(6-(4-chloro-3-ethyl-1H-pyrrolo[2,3-b]pyridin-5-yl)pyridin-2-yl)piperazin-2-one was prepared as a yellow solid (26 mg, 19%). Mass (m/z): 355.9 [M+H]⁺.

### Example 10

### 1-(4-(4-chloro-3-ethyl-1H-pyrrolo[2,3-b]pyridin-5-yl)thiazol-2-yl)piperazin-2-one

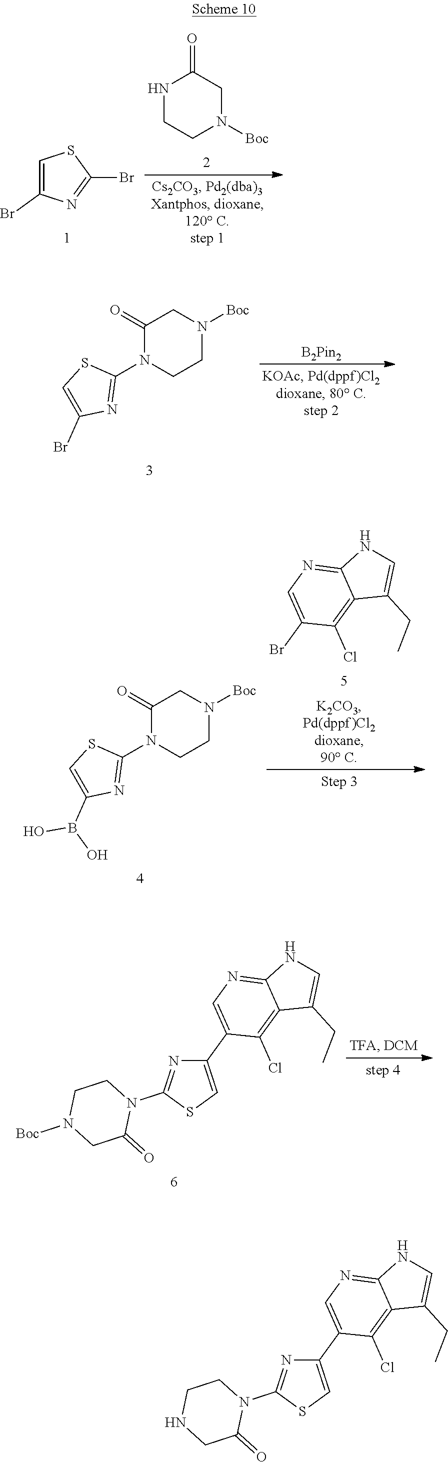

### Step 1. Preparation of tert-butyl 4-(4-bromothiazol-2-yl)-3-oxopiperazine-1-carboxylate

Following General Step H, tert-butyl 4-(4-bromothiazol-2-yl)-3-oxopiperazine-1-carboxylate was prepared as a white solid (9 g, 54%). Mass (m/z): 383.7 [M+Na]⁺.

### Step 2. Preparation of (2-(4-(tert-butoxycarbonyl)-2-oxopiperazin-1-yl)thiazol-4-yl)boronic acid

Following General Step I, (2-(4-(tert-butoxycarbonyl)-2-oxopiperazin-1-yl)thiazol-4-yl)boronic acid was prepared as a crude oil, which was used in next step without further purification.

### Step 3. Preparation of tert-butyl 4-(4-(4-chloro-3-ethyl-1H-pyrrolo[2,3-b]pyridin-5-yl)thiazol-2-yl)-3-oxopiperazine-1-carboxylate

Following General Step A, tert-butyl 4-(4-(4-chloro-3-ethyl-1H-pyrrolo[2,3-b]pyridin-5-yl)thiazol-2-yl)-3-oxopiperazine-1-carboxylate was prepared as a white solid (200 mg, 28%). Mass (m/z): 461.7 [M+H]⁺.

### Step 4. Preparation of 1-(4-(4-chloro-3-ethyl-1H-pyrrolo[2,3-b]pyridin-5-yl)thiazol-2-yl)piperazin-2-one

Following General Step B2, 1-(4-(4-chloro-3-ethyl-1H-pyrrolo[2,3-b]pyridin-5-yl)thiazol-2-yl)piperazin-2-one was prepared as a white solid (85 mg, 52%). Mass (m/z): 361.9 [M+H]⁺.

### Example 11

### 1-(4-(4-chloro-3-cyclopropyl-1H-pyrrolo[2,3-b]pyridin-5-yl)thiazol-2-yl)piperazin-2-one

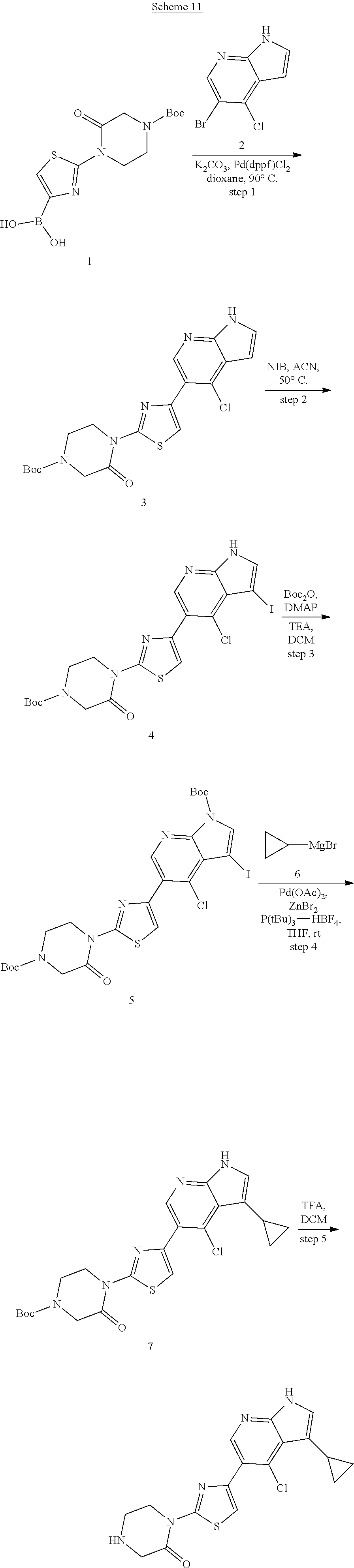

### Step 1. Preparation of tert-butyl 4-(4-(4-chloro-1H-pyrrolo[2,3-b]pyridin-5-yl)thiazol-2-yl)-3-oxopiperazine-1-carboxylate

Following General Step A, tert-butyl 4-(4-(4-chloro-1H-pyrrolo[2,3-b]pyridin-5-yl)thiazol-2-yl)-3-oxopiperazine-1-carboxylate was prepared as a white solid (400 mg, 60%). Mass (m/z): 433.8 [M+H]⁺.

### Step 2. Preparation of tert-butyl 4-(4-(4-chloro-3-iodo-1H-pyrrolo[2,3-b]pyridin-5-yl)thiazol-2-yl)-3-oxopiperazine-1-carboxylate

Following General Step F, tert-butyl 4-(4-(4-chloro-3-iodo-1H-pyrrolo[2,3-b]pyridin-5-yl)thiazol-2-yl)-3-oxopiperazine-1-carboxylate was prepared as a yellow oil (400 mg, 62%). Mass (m/z): 559.6[M+H]⁺.

### Step 3. Preparation of tert-butyl 5-(2-(4-(tert-butoxycarbonyl)-2-oxopiperazin-1-yl)thiazol-4-yl)-4-chloro-3-iodo-1H-pyrrolo[2,3-b]pyridine-1-carboxylate

Following General Step J, tert-butyl 5-(2-(4-(tert-butoxycarbonyl)-2-oxopiperazin-1-yl)thiazol-4-yl)-4-chloro-3-iodo-1H-pyrrolo[2,3-b]pyridine-1-carboxylate was prepared as a colorless oil (200 mg, 38%). Mass (m/z): 603.5 [M−55]⁺.

### Step 4. Preparation of tert-butyl 4-(4-(4-chloro-3-cyclopropyl-1H-pyrrolo[2,3-b]pyridin-5-yl)thiazol-2-yl)-3-oxopiperazine-1-carboxylate

Following General Step K, tert-butyl 4-(4-(4-chloro-3-cyclopropyl-1H-pyrrolo[2,3-b]pyridin-5-yl)thiazol-2-yl)-3-oxopiperazine-1-carboxylate was prepared as a yellow oil (40 mg, 17%). Mass (m/z): 473.9 [M+H]⁺.

### Step 5. Preparation of 1-(4-(4-chloro-3-cyclopropyl-1H-pyrrolo[2,3-b]pyridin-5-yl)thiazol-2-yl)piperazin-2-one

Following General Step B2, 1-(4-(4-chloro-3-cyclopropyl-1H-pyrrolo[2,3-b]pyridin-5-yl)thiazol-2-yl)piperazin-2-one was prepared as a white solid (8.2 mg, 25%). Mass (m/z): 373.8[M+H]⁺.

### Example 12

### 1-(3-(4-chloro-3-(2,2-difluoroethyl)-1H-pyrrolo[2,3-b]pyridin-5-yl)phenyl)piperazin-2-one

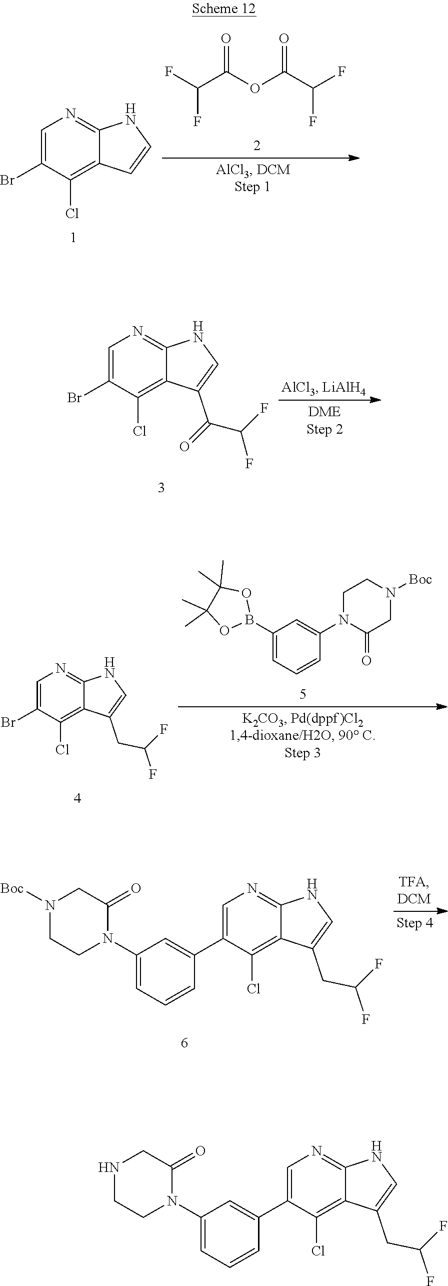

### Step 1. Preparation of 1-{5-bromo-4-chloro-1H-pyrrolo[2,3-b]pyridin-3-yl}-2,2-difluoroethanone

Following General Step L, 1-{5-bromo-4-chloro-1H-pyrrolo[2,3-b]pyridin-3-yl}-2,2-difluoroethanone was prepared as a yellow solid (200 mg, 12%). Mass (m/z): 308.7 [M+H]⁺.

### Step 2. Preparation of 5-bromo-4-chloro-3-(2,2-difluoroethyl)-1H-pyrrolo[2,3-b]pyridine

Following General Step M, 5-bromo-4-chloro-3-(2,2-difluoroethyl)-1H-pyrrolo[2,3-b]pyridine was prepared as a brown solid (50 mg, 13%). Mass (m/z): 295.0 [M+H]⁺.

### Step 3. Preparation of tert-butyl 4-(3-(4-chloro-3-(2,2-difluoroethyl)-1H-pyrrolo[2,3-b]pyridin-5-yl)phenyl)-3-oxopiperazine-1-carboxylate

Following General Step A, tert-butyl 4-(3-(4-chloro-3-(2,2-difluoroethyl)-1H-pyrrolo[2,3-b]pyridin-5-yl)phenyl)-3-oxopiperazine-1-carboxylate was prepared as a brown solid (70 mg, yield: 33%). Mass (m/z): 491.1[M+H]⁺.

### Step 4. Preparation of 1-(3-(4-chloro-3-(2,2-difluoroethyl)-1H-pyrrolo[2,3-b]pyridin-5-yl)phenyl)piperazin-2-one

Following General Step B2, 1-(3-(4-chloro-3-(2,2-difluoroethyl)-1H-pyrrolo[2,3-b]pyridin-5-yl)phenyl)piperazin-2-one was prepared as a yellow solid (5 mg, 8%). Mass (m/z): 391.0 [M+H]⁺.

### Example 13

### 1-(3-(4-chloro-3-((6-methylpyridin-3-yl)ethynyl)-1H-pyrrolo[2,3-b]pyridin-5-yl)phenyl)piperazin-2-one

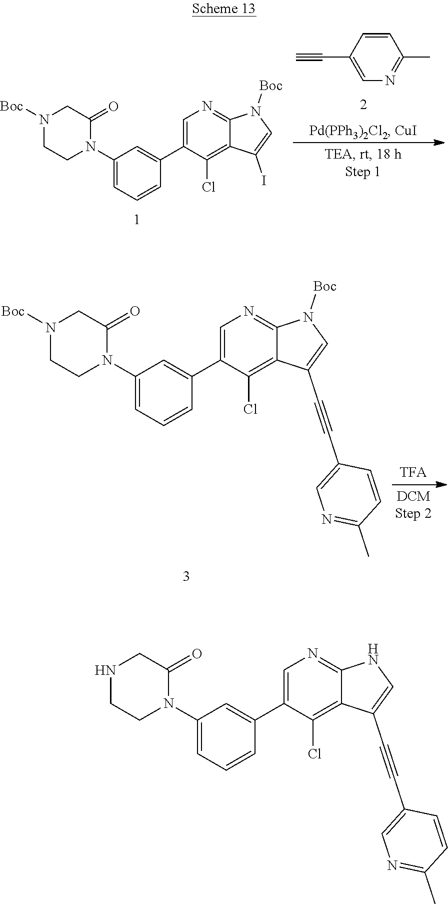

### Step 1. tert-butyl 5-(3-(4-(tert-butoxycarbonyl)-2-oxopiperazin-1-yl)phenyl)-4-chloro-3-((6-methylpyridin-3-yl)ethynyl)-1H-pyrrolo[2,3-b]pyridine-1-carboxylate

Following General Step G, tert-butyl 5-(3-(4-(tert-butoxycarbonyl)-2-oxopiperazin-1-yl)phenyl)-4-chloro-3-((6-methylpyridin-3-yl)ethynyl)-1H-pyrrolo[2,3-b]pyridine-1-carboxylate was prepared as a yellow solid (100 mg, 48%). Mass (m/z): 641.8 [M+H]⁺.

### Step 2. 1-(3-(4-chloro-3-((6-methylpyridin-3-yl)ethynyl)-1H-pyrrolo[2,3-b]pyridin-5-yl)phenyl)piperazin-2-one

Following General Step B2, 1-(3-(4-chloro-3-((6-methylpyridin-3-yl)ethynyl)-1H-pyrrolo[2,3-b]pyridin-5-yl)phenyl)piperazin-2-one was prepared as a yellow solid (50 mg, 69%). Mass (m/z): 442.1 [M+H]⁺.

### Example 14

### 1-(3-(4-chloro-3-((1-methyl-1H-1,2,4-triazol-3-yl)ethynyl)-1H-pyrrolo[2,3-b]pyridin-5-yl)phenyl)piperazin-2-one

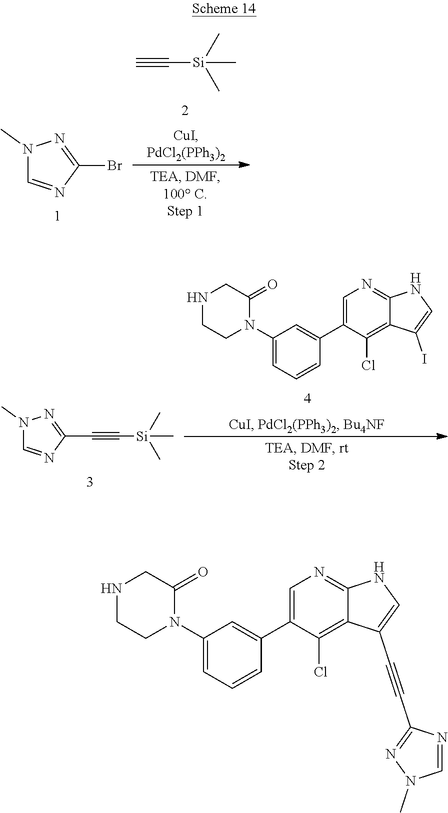

### Step 1. 1-methyl-3-((trimethylsilyl)ethynyl)-1H-1,2,4-triazole

Following General Step G, 1-methyl-3-((trimethylsilyl)ethynyl)-1H-1,2,4-triazole was prepared as a black oil (1.6 g, 69%). Mass (m/z): 180.0 [M+H]⁺.

### Step 2. 1-(3-(4-chloro-3-((1-methyl-1H-1,2,4-triazol-3-yl)ethynyl)-1H-pyrrolo[2,3-b]pyridin-5-yl)phenyl)piperazin-2-one

Following General Step G, 1-(3-(4-chloro-3-((1-methyl-1H-1,2,4-triazol-3-yl)ethynyl)-1H-pyrrolo[2,3-b]pyridin-5-yl)phenyl)piperazin-2-one was prepared as a yellow solid (11 mg, 11%). Mass (m/z): 431.7 [M+H]⁺.

### Example 15

### 1-(6-(4-chloro-3-cyclopropyl-1H-pyrrolo[2,3-b]pyridin-5-yl)pyridin-2-yl)piperazin-2-one

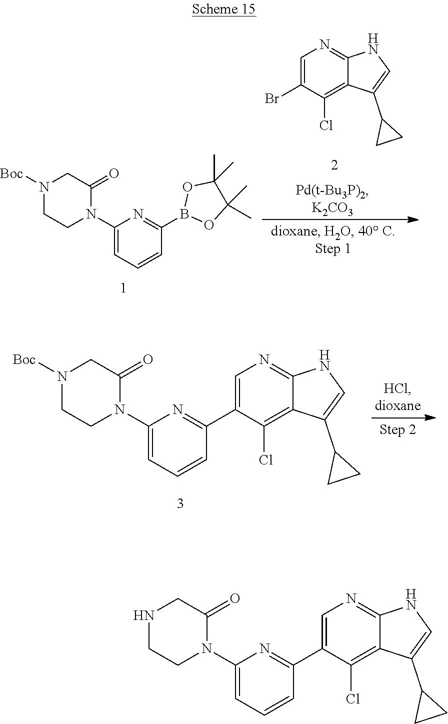 [SMILES: Clc1c(Br)cnc2[nH]cc(C3CC3)c12 ; CC(C)(C)OC(=O)N1CCN(c2cccc(B3OC(C)(C)C(C)(C)O3)n2)C(=O)C1 ; CC(C)(C)OC(=O)N1CCN(c2cccc(-c3cnc4[nH]cc(C5CC5)c4c3Cl)n2)C(=O)C1 ; O=C1CNCCN1c1cccc(-c2cnc3[nH]cc(C4CC4)c3c2Cl)n1]

### Step 1. Preparation of tert-butyl 4-(6-(4-chloro-3-cyclopropyl-1H-pyrrolo[2,3-b]pyridin-5-yl)pyridin-2-yl)-3-oxopiperazine-1-carboxylate

Following General Step A, tert-butyl 4-(6-(4-chloro-3-cyclopropyl-1H-pyrrolo[2,3-b]pyridin-5-yl)pyridin-2-yl)-3-oxopiperazine-1-carboxylate was prepared as a yellow oil (800 mg, 34% yield). Mass (m/z): 468.1 [M+H]⁺.

### Step 2. Preparation of 1-(6-(4-chloro-3-cyclopropyl-1H-pyrrolo[2,3-b]pyridin-5-yl)pyridin-2-yl)piperazin-2-one

Following General Step B1, 1-(6-(4-chloro-3-cyclopropyl-1H-pyrrolo[2,3-b]pyridin-5-yl)pyridin-2-yl)piperazin-2-one was prepared as a yellow semi-solid (4.70 g, 67% yield). Mass (m/z): 368.1 [M+H]⁺.

### Example 16

### 1-(6-(4-chloro-3-(2,2-difluoroethyl)-1H-pyrrolo[2,3-b]pyridin-5-yl)pyridin-2-yl)piperazin-2-one

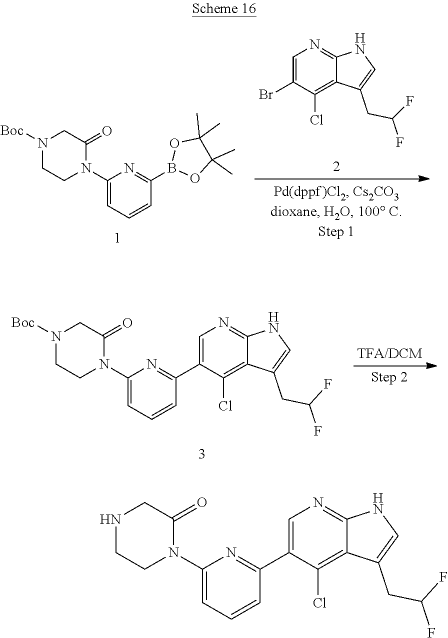 [SMILES: CC(C)(C)OC(=O)N1CCN(c2cccc(B3OC(C)(C)C(C)(C)O3)n2)C(=O)C1 ; FC(F)Cc1c[nH]c2ncc(Br)c(Cl)c12 ; CC(C)(C)OC(=O)N1CCN(c2cccc(-c3cnc4[nH]cc(CC(F)F)c4c3Cl)n2)C(=O)C1 ; O=C1CNCCN1c1cccc(-c2cnc3[nH]cc(CC(F)F)c3c2Cl)n1]

### Step 1. Preparation of tert-butyl 4-(6-(4-chloro-3-(2,2-difluoroethyl)-1H-pyrrolo[2,3-b]pyridin-5-yl)pyridin-2-yl)-3-oxopiperazine-1-carboxylate

Following General Step A, tert-butyl 4-(6-(4-chloro-3-(2,2-difluoroethyl)-1H-pyrrolo[2,3-b]pyridin-5-yl)pyridin-2-yl)-3-oxopiperazine-1-carboxylate was prepared as a yellow solid (100 mg, 12%). Mass (m/z): 492.1 [M+H]⁺.

### Step 2. Preparation of 1-(6-(4-chloro-3-(2,2-difluoroethyl)-1H-pyrrolo[2,3-b]pyridin-5-yl)pyridin-2-yl)piperazin-2-one

Following General Step B2, 1-(6-(4-chloro-3-(2,2-difluoroethyl)-1H-pyrrolo[2,3-b]pyridin-5-yl)pyridin-2-yl)piperazin-2-one was prepared as a light yellow solid (18 mg, 21%). Mass (m/z): 392.1 [M+H]⁺.

### Example 17

### 1-(3-(4-chloro-3-ethyl-1H-pyrrolo[2,3-b]pyridin-5-yl)-4-fluorophenyl)piperazin-2-one

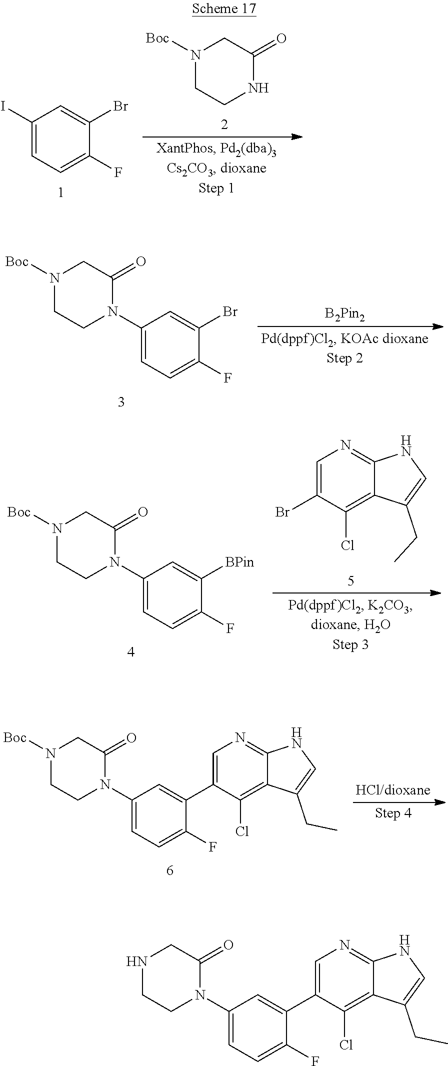

### Step 1. Preparation of [4-(3-bromo-4-fluorophenyl)-3-oxopiperazin-1-yl] tert-butyl formate

Following General Step H, [4-(3-bromo-4-fluorophenyl)-3-oxopiperazin-1-yl] tert-butyl formate was prepared as a yellow oil (3.6 g, 51%). Mass (m/z): 394.9 [M+Na]⁺.

### Step 2 & 3. Preparation of tert-butyl 4-(3-(4-chloro-3-ethyl-1H-pyrrolo[2,3-b]pyridin-5-yl)-4-fluorophenyl)-3-oxopiperazine-1-carboxylate

Following General Step I and A, tert-butyl 4-(3-(4-chloro-3-ethyl-1H-pyrrolo[2,3-b]pyridin-5-yl)-4-fluorophenyl)-3-oxopiperazine-1-carboxylate was prepared as a yellow solid (60 mg, 38%). Mass (m/z): 473.2 [M+H]⁺.

### Step 4. Preparation of 1-(3-(4-chloro-3-ethyl-1H-pyrrolo[2,3-b]pyridin-5-yl)-4-fluorophenyl)piperazin-2-one

Following General Step B1, 1-(3-(4-chloro-3-ethyl-1H-pyrrolo[2,3-b]pyridin-5-yl)-4-fluorophenyl)piperazin-2-one was prepared as a white solid (10 mg, 30%). Mass (m/z): 373.1 [M+H]⁺.

### Example 18

### 1-(4-(4-chloro-3-cyclopropyl-1H-pyrrolo[2,3-b]pyridin-5-yl)pyridin-2-yl)piperazin-2-one

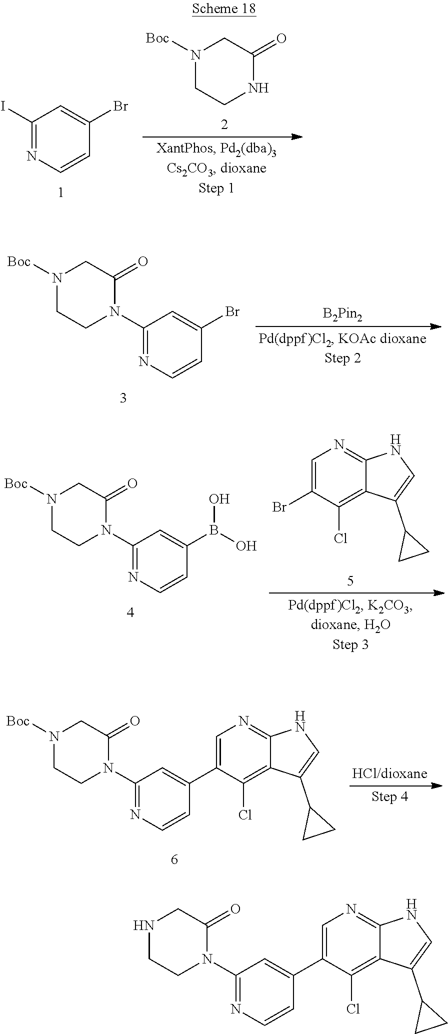

### Step 1. Preparation of tert-butyl 4-(4-bromopyridin-2-yl)-3-oxopiperazine-1-carboxylate

Following General Step H, tert-butyl 4-(4-bromopyridin-2-yl)-3-oxopiperazine-1-carboxylate was prepared as a yellow solid (2.0 g, 71%). Mass (m/z): 356.0, 358.1 [M+H]⁺.

### Step 2. Preparation of (2-(4-(tert-butoxycarbonyl)-2-oxopiperazin-1-yl)pyridin-4-yl)boronic acid

Following General Step I, (2-(4-(tert-butoxycarbonyl)-2-oxopiperazin-1-yl)pyridin-4-yl)boronic acid was prepared as a yellow solid (1.3 g, 64%). Mass (m/z): 322.0 [M+H]⁺.

### Step 3. Preparation of tert-butyl 4-(4-(4-chloro-3-cyclopropyl-1H-pyrrolo[2,3-b]pyridin-5-yl)pyridin-2-yl)-3-oxopiperazine-1-carboxylate

Following General Step A, tert-butyl 4-(4-(4-chloro-3-cyclopropyl-1H-pyrrolo[2,3-b]pyridin-5-yl)pyridin-2-yl)-3-oxopiperazine-1-carboxylate was prepared as a yellow oil (300 mg, 78%). Mass (m/z): 467.9 [M+H]⁺.

### Step 4. Preparation of 1-(4-(4-chloro-3-cyclopropyl-1H-pyrrolo[2,3-b]pyridin-5-yl)pyridin-2-yl)piperazin-2-one

Following General Step B1, 1-(4-(4-chloro-3-cyclopropyl-1H-pyrrolo[2,3-b]pyridin-5-yl)pyridin-2-yl)piperazin-2-one was prepared as a yellow solid (268 mg, 96%). Mass (m/z): 367.9 [M+H]⁺.

### Example 19

### 1-(5-(4-chloro-3-cyclopropyl-1H-pyrrolo[2,3-b]pyridin-5-yl)pyridin-3-yl)piperazin-2-one

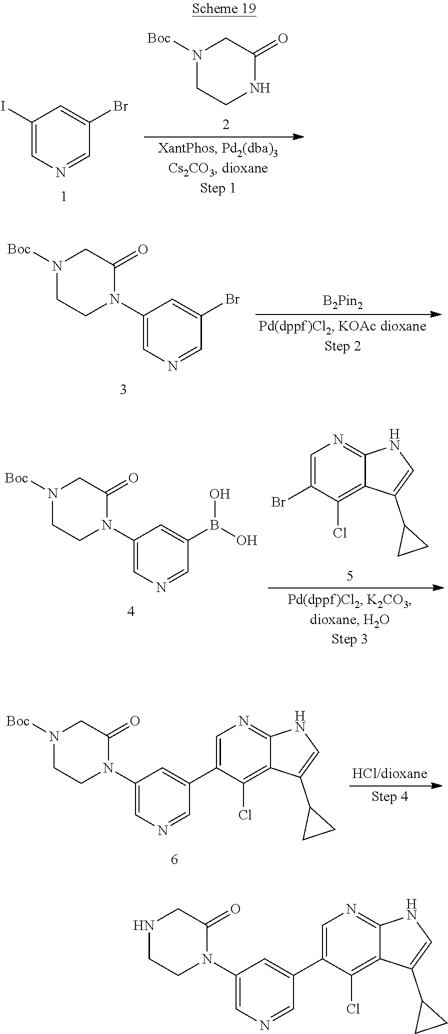

### Step 1. Preparation of tert-butyl 4-(5-bromopyridin-3-yl)-3-oxopiperazine-1-carboxylate

Following General Step H, tert-butyl 4-(5-bromopyridin-3-yl)-3-oxopiperazine-1-carboxylate was prepared as a yellow solid (2.8 g, 30%). Mass (m/z): 356.0, 358.1 [M+H]⁺.

### Step 2. Preparation of (5-(4-(tert-butoxycarbonyl)-2-oxopiperazin-1-yl)pyridin-3-yl)boronic acid

Following General Step I, (5-(4-(tert-butoxycarbonyl)-2-oxopiperazin-1-yl)pyridin-3-yl)boronic acid was prepared as a yellow solid (850 mg, 30%). Mass (m/z): 321.9 [M+H]⁺.

### Step 3. Preparation of tert-butyl 4-(5-(4-chloro-3-cyclopropyl-1H-pyrrolo[2,3-b]pyridin-5-yl)pyridin-3-yl)-3-oxopiperazine-1-carboxylate

Following General Step A, tert-butyl 4-(5-(4-chloro-3-cyclopropyl-1H-pyrrolo[2,3-b]pyridin-5-yl)pyridin-3-yl)-3-oxopiperazine-1-carboxylate was prepared as a yellow solid (200 mg, 52%). Mass (m/z): 468.0 [M+H]⁺.

### Step 4. Preparation of 1-(5-(4-chloro-3-cyclopropyl-1H-pyrrolo[2,3-b]pyridin-5-yl)pyridin-3-yl)piperazin-2-one

Following General Step B1, 1-(5-(4-chloro-3-cyclopropyl-1H-pyrrolo[2,3-b]pyridin-5-yl)pyridin-3-yl)piperazin-2-one was prepared as a yellow solid (200 mg, 14%). Mass (m/z): 368.0 [M+H]⁺.

### Example 20

### 1-(4-(4-chloro-3-ethyl-1H-pyrrolo[2,3-b]pyridin-5-yl)-5-methylthiazol-2-yl)piperazin-2-one

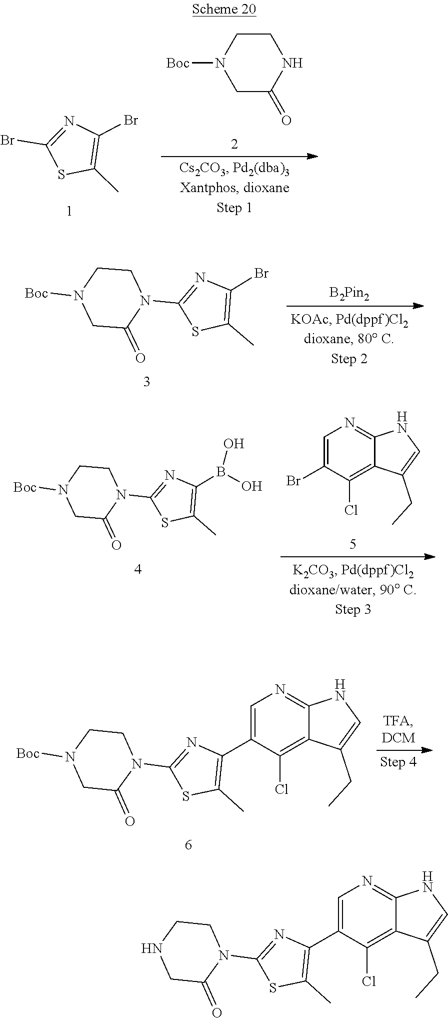

### Step 1. Preparation of tert-butyl 4-(4-bromo-5-methylthiazol-2-yl)-3-oxopiperazine-1 carboxylate

Following General Step H, tert-butyl 4-(4-bromo-5-methylthiazol-2-yl)-3-oxopiperazine-1-carboxylate was prepared as a yellow solid (800 mg, 49%). Mass (m/z): 376.1, 378.0 [M+H]⁺.

### Step 2 & 3. Preparation of tert-butyl 4-(4-(4-chloro-3-ethyl-1H-pyrrolo[2,3-b]pyridin-5-yl)-5-methylthiazol-2-yl)-3-oxopiperazine-1-carboxylate

Following General Step I and A, tert-butyl 4-(4-(4-chloro-3-ethyl-1H-pyrrolo[2,3-b]pyridin-5-yl)-5-methylthiazol-2-yl)-3-oxopiperazine-1-carboxylate was prepared as a yellow solid (1.0 g, 69%). Mass (m/z): 476.1 [M+H]⁺.

### Step 4. Preparation of 1-(4-(4-chloro-3-ethyl-1H-pyrrolo[2,3-b]pyridin-5-yl)-5-methylthiazol-2-yl)piperazin-2-one

Following General Step B2, 1-(4-(4-chloro-3-ethyl-1H-pyrrolo[2,3-b]pyridin-5-yl)-5-methylthiazol-2-yl)piperazin-2-one was prepared as a white solid (68 mg, 8%). Mass (m/z): 376.1 [M+H]⁺.

### Example 21

### 1-(3-(3-ethyl-1H-pyrrolo[2,3-b]pyridin-5-yl)-4-(hydroxymethyl)phenyl)piperazin-2-one

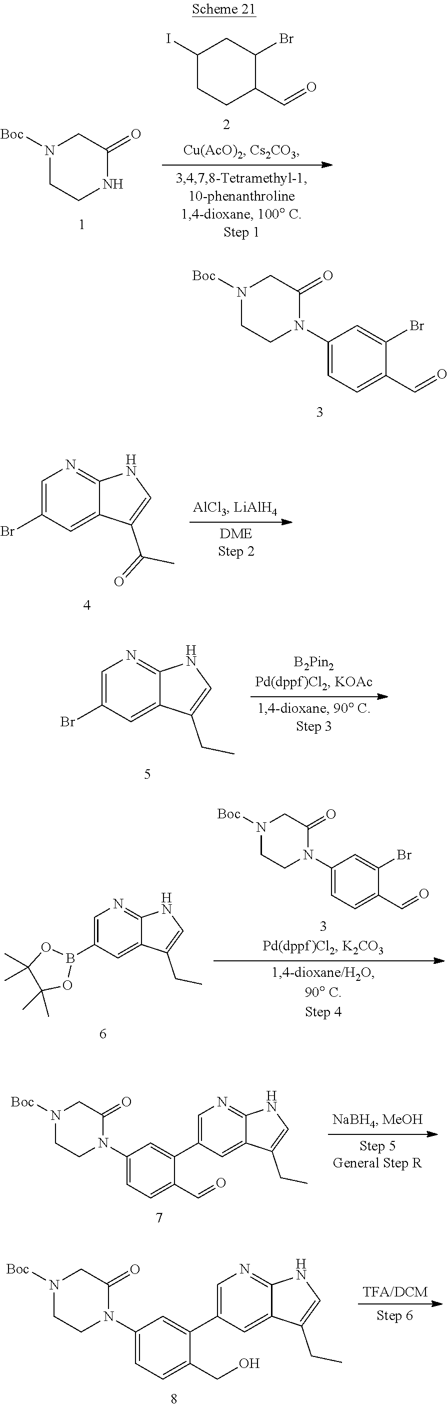

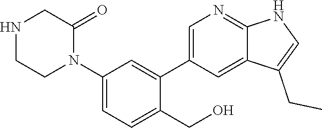 [SMILES: CCc1c[nH]c2ncc(-c3cc(N4CCNCC4=O)ccc3CO)cc12]

### Step 1. Preparation of [4-(3-bromo-4-formylphenyl)-3-oxopiperazin-1-yl] tert-butyl formate

Following General Step H, [4-(3-bromo-4-formylphenyl)-3-oxopiperazin-1-yl] tert-butyl formate was prepared as a yellow oil (1.2 g, 16%). Mass (m/z): 405.1 [M+Na]⁺.

### Step 2. Preparation of 5-bromo-3-ethyl-1H-pyrrolo[2,3-b]pyridine

Following General Step M, 5-bromo-3-ethyl-1H-pyrrolo[2,3-b]pyridine was prepared as a brown solid (4.0 g, 85%). Mass (m/z): 224.9 [M+H]⁺.

### Step 3 & 4. Preparation of tert-butyl 4-(3-(3-ethyl-1H-pyrrolo[2,3-b]pyridin-5-yl)-4-formylphenyl)-3-oxopiperazine-1-carboxylate

Following General Step I and A, tert-butyl 4-(3-(3-ethyl-1H-pyrrolo[2,3-b]pyridin-5-yl)-4-formylphenyl)-3-oxopiperazine-1-carboxylate was prepared as a yellow solid (300 mg, 24%). Mass (m/z): 449.2 [M+H]⁺.

### Step 5. (General Step R) Preparation of tert-butyl 4-(3-(3-ethyl-1H-pyrrolo[2,3-b]pyridin-5-yl)-4-(hydroxymethyl)phenyl)-3-oxopiperazine-1-carboxylate

To a solution of tert-butyl 4-(3-(3-ethyl-1H-pyrrolo[2,3-b]pyridin-5-yl)-4-formylphenyl)-3-oxopiperazine-1-carboxylate (210 mg, 0.47 mmol) in MeOH (10 mL) was added NaBH₄ (35 mg, 0.92 mmol) at 0° C. The resulting mixture was stirred at 25° C. for 2 hours, and then concentrated under vacuum. The crude residue was purified by Combiflash (PE/EA=4:1) to give tert-butyl 4-(3-(3-ethyl-1H-pyrrolo[2,3-b]pyridin-5-yl)-4-(hydroxymethyl)phenyl)-3-oxopiperazine-1-carboxylate (140 mg, 66%) as a yellow solid. Mass (m/z): 451.2 [M+H]⁺.

### Step 6. Preparation of 1-(3-(3-ethyl-1H-pyrrolo[2,3-b]pyridin-5-yl)-4-(hydroxymethyl)phenyl)piperazin-2-one

Following General Step B2, 1-(3-(3-ethyl-1H-pyrrolo[2,3-b]pyridin-5-yl)-4-(hydroxymethyl)phenyl)piperazin-2-one was prepared as a light yellow solid (25 mg, 22%). Mass (m/z): 351.2 [M+H]⁺.

### Example 22

### 1-(3-(4-chloro-3-ethyl-1H-pyrrolo[2,3-b]pyridin-5-yl)phenyl)-3-(hydroxymethyl)piperidin-2-one

### Example 23

### (R)-1-(3-(4-chloro-3-ethyl-1H-pyrrolo[2,3-b]pyridin-5-yl)phenyl)-3-(hydroxymethyl)piperidin-2-one

### Example 24

### (S)-1-(3-(4-chloro-3-ethyl-1H-pyrrolo[2,3-b]pyridin-5-yl)phenyl)-3-(hydroxymethyl)piperidin-2-one

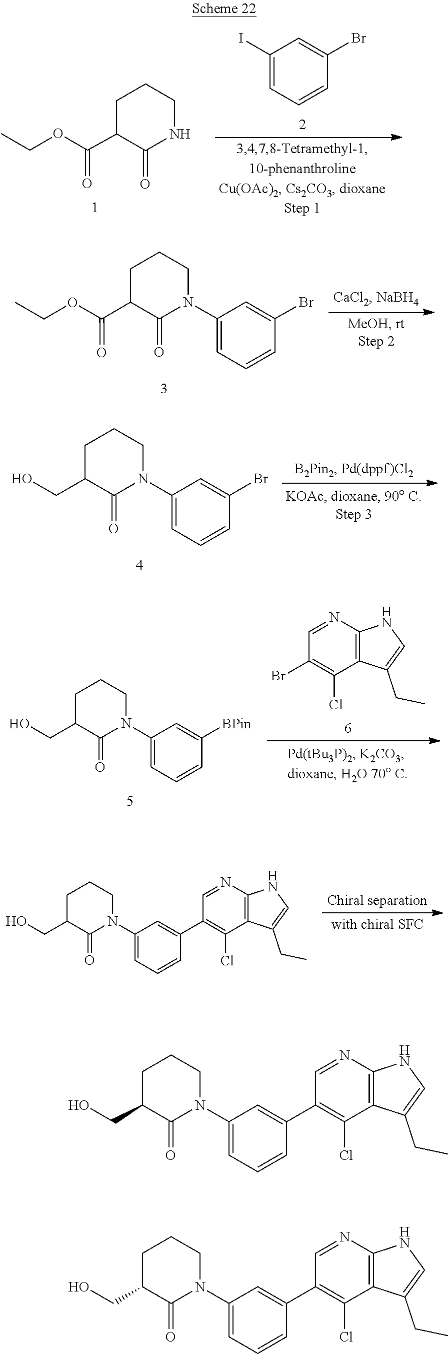

### Step 1. Preparation of ethyl 1-(3-bromophenyl)-2-oxopiperidine-3-carboxylate

Following General Step H, ethyl 1-(3-bromophenyl)-2-oxopiperidine-3-carboxylate was prepared as a yellow oil (5.34 g, 56%). Mass (m/z): 325.8 [M+H]⁺.

### Step 2. Preparation of 1-(3-bromophenyl)-3-(hydroxymethyl)piperidin-2-one

Following General Step R, 1-(3-bromophenyl)-3-(hydroxymethyl)piperidin-2-one was prepared as a yellow oil (3.9 g, 83%). Mass (m/z): 284.1 [M+H]⁺.

### Step 3. Preparation of 3-(hydroxymethyl)-1-(3-(4,4,5,5-tetramethyl-1,3,2-dioxaborolan-2-yl)phenyl)piperidin-2-one

Following General Step I, 3-(hydroxymethyl)-1-(3-(4,4,5,5-tetramethyl-1,3,2-dioxaborolan-2-yl)phenyl)piperidin-2-one was prepared as a yellow oil (5.07 g, 93%). Mass (m/z): 332.1 [M+H]⁺.

### Step 4. Preparation of 1-(3-(4-chloro-3-ethyl-1H-pyrrolo[2,3-b]pyridin-5-yl)phenyl)-3-(hydroxymethyl)piperidin-2-one, (R)-1-(3-(4-chloro-3-ethyl-1H-pyrrolo[2,3-b]pyridin-5-yl)phenyl)-3-(hydroxymethyl)piperidin-2-one and (S)-1-(3-(4-chloro-3-ethyl-1H-pyrrolo[2,3-b]pyridin-5-yl)phenyl)-3-(hydroxymethyl)piperidin-2-one

Following General Step A, 1-(3-(4-chloro-3-ethyl-1H-pyrrolo[2,3-b]pyridin-5-yl)phenyl)-3-(hydroxymethyl)piperidin-2-one was prepared as a white solid (200 mg, 5.8%). Mass (m/z): 384.2 [M+H]⁺.

Racemic 1-(3-(4-chloro-3-ethyl-1H-pyrrolo[2,3-b]pyridin-5-yl)phenyl)-3-(hydroxymethyl)piperidin-2-one was further separated with chiral SFC [SP-120-10-C18-BIO-C18, 250×50 mm, 20% IPA (DEA) in CO₂] to give (R)-1-(3-(4-chloro-3-ethyl-1H-pyrrolo[2,3-b]pyridin-5-yl)phenyl)-3-(hydroxymethyl)piperidin-2-one as a white solid (35 mg) and (S)-1-(3-(4-chloro-3-ethyl-1H-pyrrolo[2,3-b]pyridin-5-yl)phenyl)-3-(hydroxymethyl)piperidin-2-one as a white solid (34 mg).

### Example 25

### 1-(3-(4-chloro-3-ethyl-1H-pyrrolo[2,3-b]pyridin-5-yl)phenyl)-4-(2-methoxyacetyl)piperazin-2-one

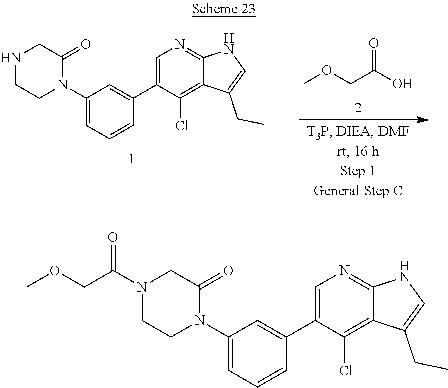

### Step 1. (General Step C) Preparation of 1-(3-(4-chloro-3-ethyl-1H-pyrrolo[2,3-b]pyridin-5-yl)phenyl)-4-(2-methoxyacetyl)piperazin-2-one

To a solution of 1-(3-(4-chloro-3-ethyl-1H-pyrrolo[2,3-b]pyridin-5-yl)phenyl)piperazin-2-one (150 mg, 0.422 mmol) in DMF (5.0 mL) were added methoxyacetic acid (57 mg, 0.634 mmol), DIEA (164 mg, 1.26 mmol) and T₃P (538 mg, 0.854 mmol, 50% in EtOAc). The resulting solution was stirred at rt for 16 hrs, and then diluted with H₂O. The aqueous media was extracted with EtOAc (50 mL×3). The combined organic layers were washed with brine, dried over Na₂SO₄, and filtered. The filtrate was concentrated under reduced pressure. The residue was purified via Prep-TLC (CH₂Cl₂/MeOH=15:1) to give 1-(3-(4-chloro-3-ethyl-1H-pyrrolo[2,3-b]pyridin-5-yl)phenyl)-4-(2-methoxyacetyl)piperazin-2-one as a white solid (66.8 mg, 36%). Mass (m/z): 426.9 [M+H]⁺.

### Example 26

### 1-(3-(4-chloro-3-ethyl-1H-pyrrolo[2,3-b]pyridin-5-yl)phenyl)-4-(2-morpholinoacetyl)piperazin-2-one

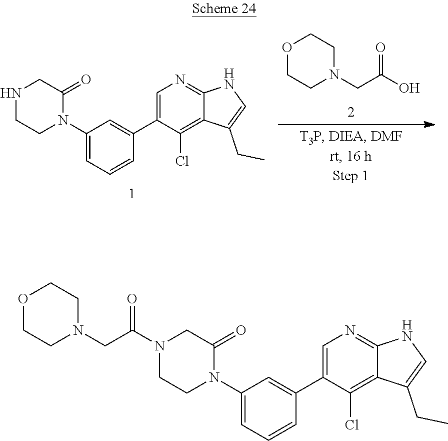 [SMILES: CCc1c[nH]c2ncc(-c3cccc(N4CCNCC4=O)c3)c(Cl)c12 ; O=C(O)CN1CCOCC1 ; CCc1c[nH]c2ncc(-c3cccc(N4CCN(C(=O)CN5CCOCC5)CC4=O)c3)c(Cl)c12]

### Step 1. Preparation of 1-(3-(4-chloro-3-ethyl-1H-pyrrolo[2,3-b]pyridin-5-yl)phenyl)-4-(2-morpholinoacetyl)piperazin-2-one

Following General Step C, 1-(3-(4-chloro-3-ethyl-1H-pyrrolo[2,3-b]pyridin-5-yl)phenyl)-4-(2-morpholinoacetyl)piperazin-2-one was prepared as a white solid (78.5 mg, 37%). Mass (m/z): 481.9 [M+H]⁺.

### Example 27

### Methyl 4-(3-(4-chloro-3-ethyl-1H-pyrrolo[2,3-b]pyridin-5-yl)phenyl)-3-oxopiperazine-1-carboxylate

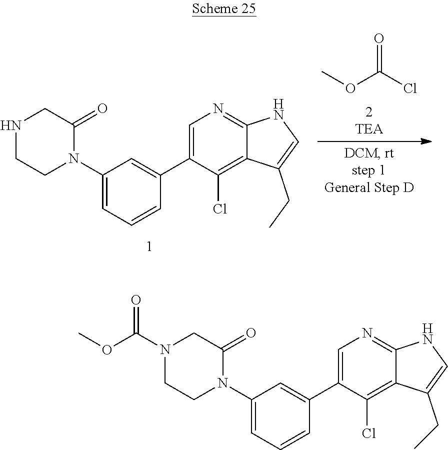

### Step 1. (General Step D) Preparation of methyl 4-(3-(4-chloro-3-ethyl-1H-pyrrolo[2,3-b]pyridin-5-yl)phenyl)-3-oxopiperazine-1-carboxylate

To a solution of 1-(3-(4-chloro-3-ethyl-1H-pyrrolo[2,3-b]pyridin-5-yl)phenyl)piperazin-2-one (150 mg, 0.42 mmol) in DCM (5 mL) were added TEA (86 mg, 0.84 mmol) and methyl carbonochloridate (44 mg, 0.46 mmol) at 0° C. The reaction mixture was stirred at rt under N₂ for 16 hrs. After the reaction completed, H₂O (20 mL) was added to the reaction mixture, and then extracted with DCM (20 mL×3). The combined organic layers were washed with brine (20 mL×2), and then dried over with anhydrous Na₂SO₄. After filtration, the solution was concentration under vacuum, and the crude product was purified by Combiflash (DCM/MeOH=0˜2.5%) to give the product methyl 4-(3-(4-chloro-3-ethyl-1H-pyrrolo[2,3-b]pyridin-5-yl)phenyl)-3-oxopiperazine-1-carboxylate (Example 5) as a white solid (26 mg, 15%). Mass (m/z): 412.8 [M+H]⁺.

### Example 28

### 1-(3-(4-chloro-3-ethyl-1H-pyrrolo[2,3-b]pyridin-5-yl)phenyl)-4-(2-methoxyethyl)piperazin-2-one

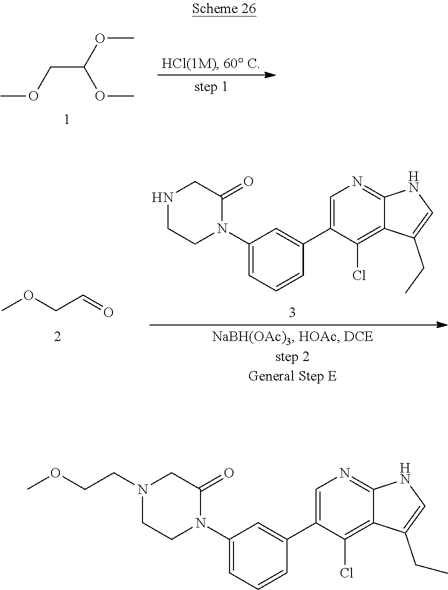

### Step 1. & Step 2. (General Step E) Preparation of 1-(3-(4-chloro-3-ethyl-1H-pyrrolo[2,3-b]pyridin-5-yl)phenyl)-4-(2-methoxyethyl)piperazin-2-one

A solution of 1,1,2-trimethoxyethane (338 mg, 2.818 mmol) in 1N HCl solution (5 mL) was stirred at 50° C. for 3 hrs. The solution was cooled to rt and extracted with DCE (5 mL×2). The combined organic layers were washed with brine (20 mL×2), and dried over Na₂SO₄. The solution was added to a solution of 1-(3-(4-chloro-3-ethyl-1H-pyrrolo[2,3-b]pyridin-5-yl)phenyl)piperazin-2-one (50 mg, 0.14 mmol) in DCE (5 mL), followed by the addition of AcOH (two drops) and sodium triacetoxyborohydride (89 mg, 0.422 mmol). The reaction mixture was stirred at rt under N₂ for 3 hrs, and then concentrated. The residue was purified by prep-TLC (DCM:MeOH=20:1) to give 1-(3-(4-chloro-3-ethyl-1H-pyrrolo[2,3-b]pyridin-5-yl)phenyl)-4-(2-methoxyethyl)piperazin-2-one as a yellow solid (66.8 mg, 36%). Mass (m/z): 412.9 [M+H]⁺.

### Example 29

### 1-(3-(4-chloro-3-(pyridin-3-ylethynyl)-1H-pyrrolo[2,3-b]pyridin-5-yl)phenyl)-4-(2-methoxyacetyl)piperazin-2-one

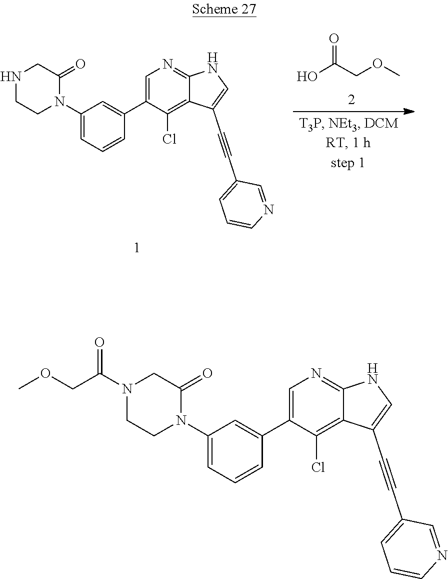

### Step 1. Preparation of 1-(3-(4-chloro-3-(pyridin-3-ylethynyl)-1H-pyrrolo[2,3-b]pyridin-5-yl)phenyl)-4-(2-methoxyacetyl)piperazin-2-one

Following General Step C, 1-(3-(4-chloro-3-(pyridin-3-ylethynyl)-1H-pyrrolo[2,3-b]pyridin-5-yl)phenyl)-4-(2-methoxyacetyl)piperazin-2-one was prepared as a white solid (15 mg, 42%). Mass (m/z): 500.1 [M+H]⁺.

### Example 30

### 1-(3-(4-chloro-3-(pyridin-3-ylethynyl)-1H-pyrrolo[2,3-b]pyridin-5-yl)phenyl)-4-(dimethylglycyl)piperazin-2-one

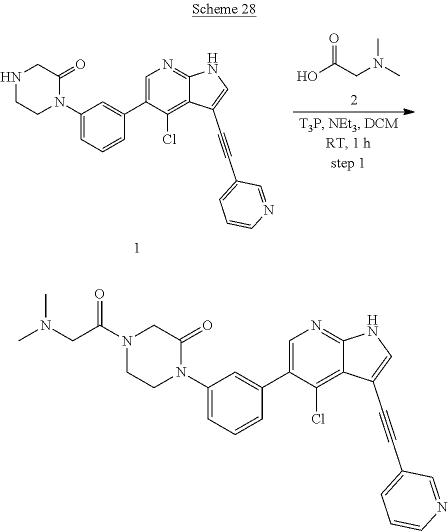

### Step 1. Preparation of 1-(3-(4-chloro-3-(pyridin-3-ylethynyl)-1H-pyrrolo[2,3-b]pyridin-5-yl)phenyl)-4-(dimethylglycyl)piperazin-2-one

Following General Step C, 1-(3-(4-chloro-3-(pyridin-3-ylethynyl)-1H-pyrrolo[2,3-b]pyridin-5-yl)phenyl)-4-(dimethylglycyl)piperazin-2-one was prepared as a white solid (10 mg, 27%). Mass (m/z): 513.2 [M+H]⁺.

### Example 31

### 1-(3-(4-chloro-3-ethyl-1H-pyrrolo[2,3-b]pyridin-5-yl)phenyl)-4-(dimethylglycyl)piperazin-2-one

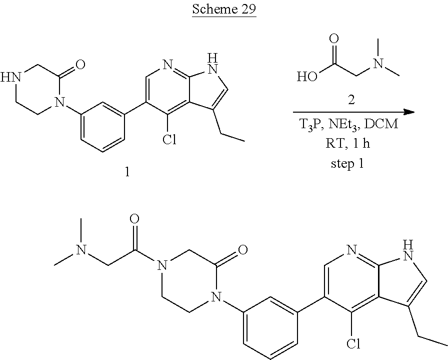

### Step 1. Preparation of 1-(3-(4-chloro-3-ethyl-1H-pyrrolo[2,3-b]pyridin-5-yl)phenyl)-4-(dimethylglycyl)piperazin-2-one

Following General Step C, 1-(3-(4-chloro-3-ethyl-1H-pyrrolo[2,3-b]pyridin-5-yl)phenyl)-4-(dimethylglycyl)piperazin-2-one was prepared as a white solid (15 mg, 18%). Mass (m/z): 440.2 [M+H]⁺.

### Example 32

### 1-(3-(4-chloro-3-cyclopropyl-1H-pyrrolo[2,3-b] pyridin-5-yl) phenyl)-4-(2-methoxyacetyl) piperazin-2-one

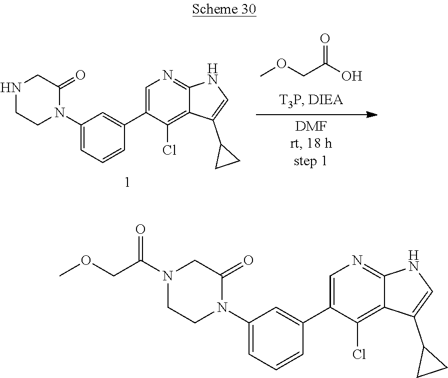

### Step 1. Preparation of 1-(3-(4-chloro-3-cyclopropyl-1H-pyrrolo[2,3-b] pyridin-5-yl) phenyl)-4-(2-methoxyacetyl) piperazin-2-one

Following General Step C, 1-(3-(4-chloro-3-cyclopropyl-1H-pyrrolo[2,3-b] pyridin-5-yl) phenyl)-4-(2-methoxyacetyl) piperazin-2-one was prepared as a white solid (25 mg, 7%). MS: (m/z)=438.9 (M+1, ESI+).

### Example 33

### 4-(2-((1s,3s)-adamantan-1-yl)acetyl)-1-(3-(4-chloro-3-ethyl-1H-pyrrolo[2,3-b]pyridin-5-yl)phenyl)piperazin-2-one

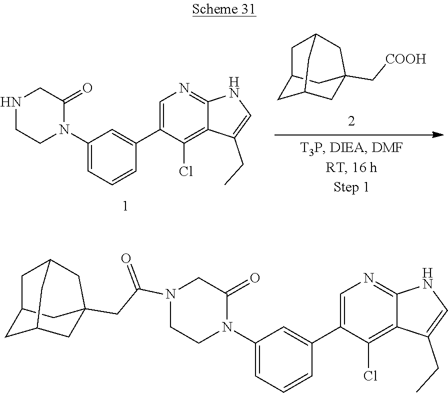

### Step 1. Preparation of 4-(2-((1s,3s)-adamantan-1-yl)acetyl)-1-(3-(4-chloro-3-ethyl-1H-pyrrolo[2,3-b]pyridin-5-yl)phenyl)piperazin-2-one

Following General Step C, 4-(2-((1s,3s)-adamantan-1-yl)acetyl)-1-(3-(4-chloro-3-ethyl-1H-pyrrolo[2,3-b]pyridin-5-yl)phenyl)piperazin-2-one was prepared as a white solid (4.0 mg, 6.6%). Mass (m/z): 530.8 [M+H]⁺.

### Example 34

### 2-((3r,5r,7r)-adamantan-1-yl)-N-(4-(4-(3-(4-chloro-3-ethyl-1H-pyrrolo[2,3-b]pyridin-5-yl)phenyl)-3-oxopiperazin-1-yl)-4-oxobutyl)acetamide

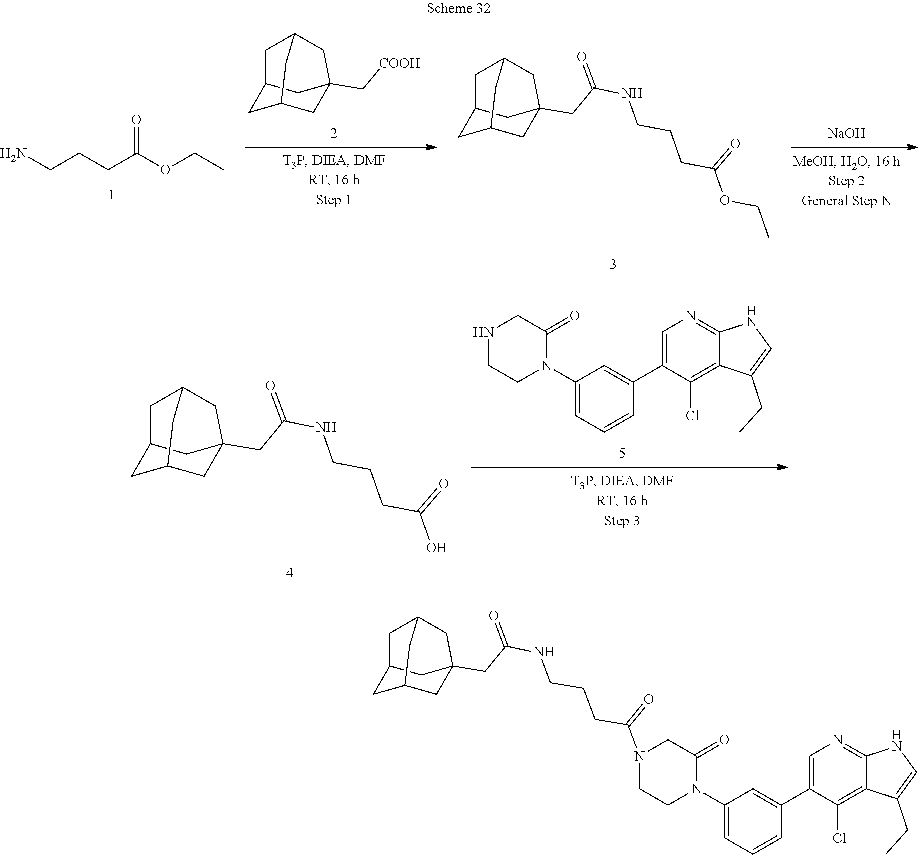

### Step 1. Preparation of ethyl 4-(2-((3r,5r,7r)-adamantan-1-yl)acetamido)butanoate

Following General Step C, ethyl 4-(2-((3r,5r,7r)-adamantan-1-yl)acetamido)butanoate was prepared as a colorless oil (240 mg, 60%). Mass (m/z): 308.0 [M+H]⁺.

### Step 2. (General Step N) Preparation of 4-(2-((3r,5r,7r)-adamantan-1-yl)acetamido)butanoic acid

To a solution of ethyl 4-(2-((3r,5r,7r)-adamantan-1-yl)acetamido)butanoate (240 mg, 0.78 mmol) in the mixed solvent of MeOH (4 mL) and H₂O (1 mL) was added NaOH (312 mg, 7.8 mmol). The resulting solution was stirred at ambient temperature for 16 hours, and then diluted with water (10 mL). The aqueous solution was acidified to pH 3-4 with aqueous HCl solution (2M), and then extracted with EA (10 mL×3). The combined organic layers were washed with brine, dried over Na₂SO₄, and filtered. The filtrate was concentrated under reduced pressure to give 4-(2-((3r,5r,7r)-adamantan-1-yl)acetamido)butanoic acid as a white solid (210 mg, 77%). Mass (m/z): 280.1 [M+H]⁺.

### Step 3. Preparation of 2-((3r,5r,7r)-adamantan-1-yl)-N-(4-(4-(3-(4-chloro-3-ethyl-1H-pyrrolo[2,3-b]pyridin-5-yl)phenyl)-3-oxopiperazin-1-yl)-4-oxobutyl)acetamide

Following General Step C, 2-((3r,5r,7r)-adamantan-1-yl)-N-(4-(4-(3-(4-chloro-3-ethyl-1H-pyrrolo[2,3-b]pyridin-5-yl)phenyl)-3-oxopiperazin-1-yl)-4-oxobutyl)acetamide was prepared as a white solid (15 mg, 10%). Mass (m/z): 615.8 [M+H]⁺.

### Example 35

### 1-(3-(4-chloro-3-(pyridin-2-ylethynyl)-1H-pyrrolo[2,3-b]pyridin-5-yl)phenyl)-4-(2-methoxyacetyl)piperazin-2-one

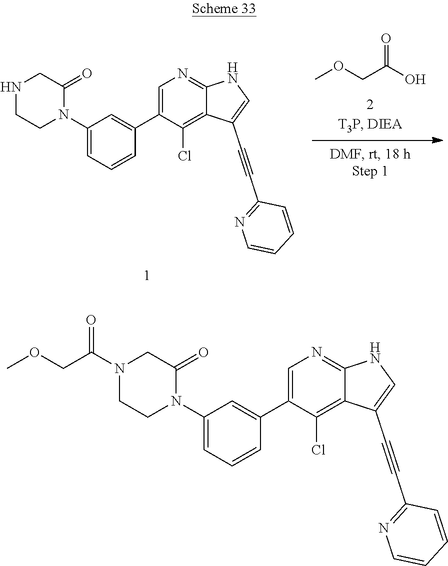

### Step 1. 1-(3-(4-chloro-3-(pyridin-2-ylethynyl)-1H-pyrrolo[2,3-b]pyridin-5-yl)phenyl)-4-(2-methoxyacetyl)piperazin-2-one

Following General Step C, 1-(3-(4-chloro-3-(pyridin-2-ylethynyl)-1H-pyrrolo[2,3-b]pyridin-5-yl)phenyl)-4-(2-methoxyacetyl)piperazin-2-one was prepared as a white solid (2.3 mg, 3.8%). Mass (m/z): 500.1 [M+H]⁺.

### Example 36

### 1-(3-(4-chloro-3-((1-methyl-1H-1,2,4-triazol-3-yl)ethynyl)-1H-pyrrolo[2,3-b]pyridin-5-yl)phenyl)-4-(2-methoxyacetyl)piperazin-2-one

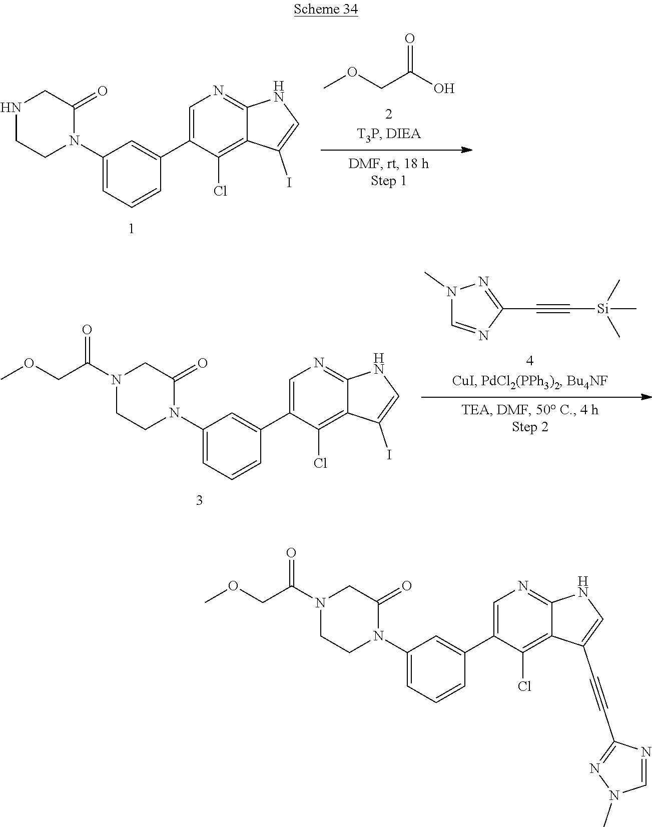

### Step 1. 1-(3-(4-chloro-3-iodo-1H-pyrrolo[2,3-b]pyridin-5-yl)phenyl)-4-(2-methoxyacetyl)piperazin-2-one

Following General Step C, 1-(3-(4-chloro-3-iodo-1H-pyrrolo[2,3-b]pyridin-5-yl)phenyl)-4-(2-methoxyacetyl)piperazin-2-one was prepared as a yellow solid (110 mg, 90%). Mass (m/z): 524.7 [M+H]⁺.

### Step 2. 1-(3-(4-chloro-3-((1-methyl-1H-1,2,4-triazol-3-yl)ethynyl)-1H-pyrrolo[2,3-b]pyridin-5-yl)phenyl)-4-(2-methoxyacetyl)piperazin-2-one

Following General Step G, 1-(3-(4-chloro-3-((1-methyl-1H-1,2,4-triazol-3-yl)ethynyl)-1H-pyrrolo[2,3-b]pyridin-5-yl)phenyl)-4-(2-methoxyacetyl)piperazin-2-one was prepared as a yellow solid (4 mg, 24%). Mass (m/z): 504.0 [M+H]⁺.

### Example 37

### 1-(3-(4-chloro-3-(2,2-difluoroethyl)-1H-pyrrolo[2,3-b]pyridin-5-yl)phenyl)-4-(2-methoxyethyl)piperazin-2-one

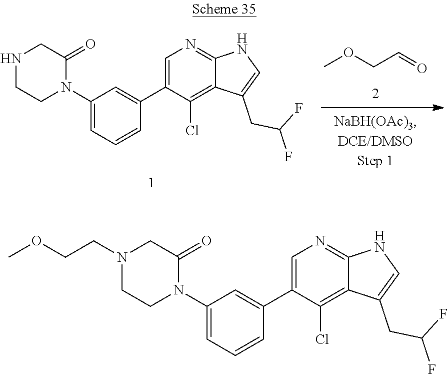

### Step 1. Preparation of 1-(3-(4-chloro-3-(2,2-difluoroethyl)-1H-pyrrolo[2,3-b]pyridin-5-yl)phenyl)-4-(2-methoxyethyl)piperazin-2-one

Following General Step E, 1-(3-(4-chloro-3-(2,2-difluoroethyl)-1H-pyrrolo[2,3-b]pyridin-5-yl)phenyl)-4-(2-methoxyethyl)piperazin-2-one was prepared as a white solid (120 mg, 25%). Mass (m/z): 449.1 [M+H]⁺.

### Example 38

### 1-(3-(4-chloro-3-(2,2-difluoroethyl)-1H-pyrrolo[2,3-b]pyridin-5-yl)phenyl)-4-(2-methoxyacetyl)piperazin-2-one

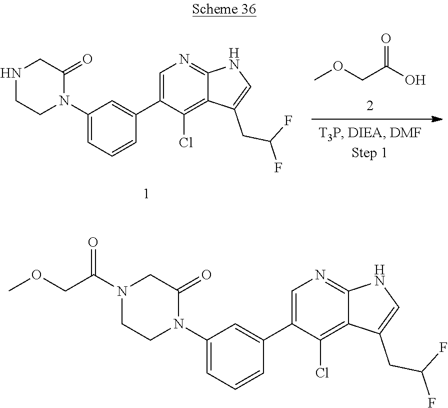

### Step 1. 1-(3-(4-chloro-3-(2,2-difluoroethyl)-1H-pyrrolo[2,3-b]pyridin-5-yl)phenyl)-4-(2-methoxyacetyl)piperazin-2-one

Following General Step C, 1-(3-(4-chloro-3-(2,2-difluoroethyl)-1H-pyrrolo[2,3-b]pyridin-5-yl)phenyl)-4-(2-methoxyacetyl)piperazin-2-one was prepared as a light yellow solid (11 mg, 18%). Mass (m/z): 463.1 [M+H]⁺.

### Example 39

### 1-(3-(4-chloro-3-cyclopropyl-1H-pyrrolo[2,3-b]pyridin-5-yl)phenyl)-4-(2-methoxyethyl)piperazin-2-one

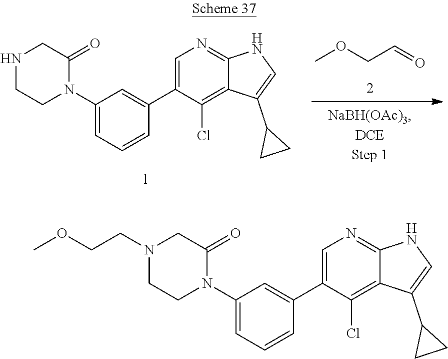

### Step 1. Preparation of 1-(3-(4-chloro-3-cyclopropyl-1H-pyrrolo[2,3-b]pyridin-5-yl)phenyl)-4-(2-methoxyethyl)piperazin-2-one

Following General Step E, 1-(3-(4-chloro-3-cyclopropyl-1H-pyrrolo[2,3-b]pyridin-5-yl)phenyl)-4-(2-methoxyethyl)piperazin-2-one was prepared as a white solid (8 mg, 2%). Mass (m/z): 425.0 [M+H]⁺.

### Example 40

### 1-(4-((dimethylamino)methyl)-3-(3-ethyl-1H-pyrrolo[2,3-b]pyridin-5-yl)phenyl)piperazin-2-one

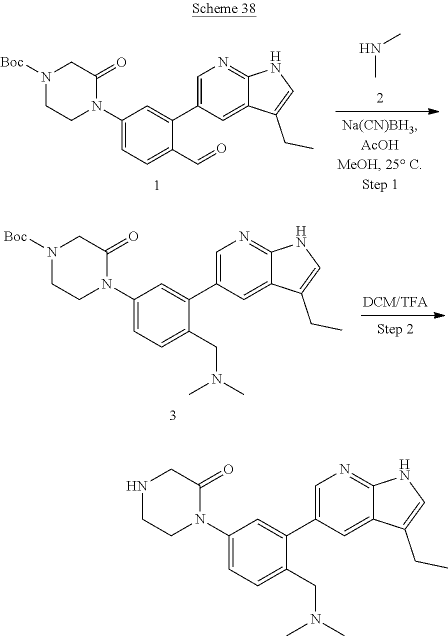

### Step 1. Preparation of tert-butyl 4-(4-((dimethylamino)methyl)-3-(3-ethyl-1H-pyrrolo[2,3-b]pyridin-5-yl)phenyl)-3-oxopiperazine-1-carboxylate

Following General Step E, tert-butyl 4-(4-((dimethylamino)methyl)-3-(3-ethyl-1H-pyrrolo[2,3-b]pyridin-5-yl)phenyl)-3-oxopiperazine-1-carboxylate was prepared as a light yellow solid (130 mg, 55%). Mass (m/z): 478.3 [M+H]⁺.

### Step 2. Preparation of 1-(4-((dimethylamino)methyl)-3-(3-ethyl-1H-pyrrolo[2,3-b]pyridin-5-yl)phenyl)piperazin-2-one

Following General Step B2, 1-(4-((dimethylamino)methyl)-3-(3-ethyl-1H-pyrrolo[2,3-b]pyridin-5-yl)phenyl)piperazin-2-one was prepared as a light yellow solid (20 mg, 19%). Mass (m/z): 378.2 [M+H]⁺.

### Example 41

### 1-(3-(3-ethyl-1H-pyrrolo[2,3-b]pyridin-5-yl)-4-((methylamino)methyl)phenyl)piperazin-2-one

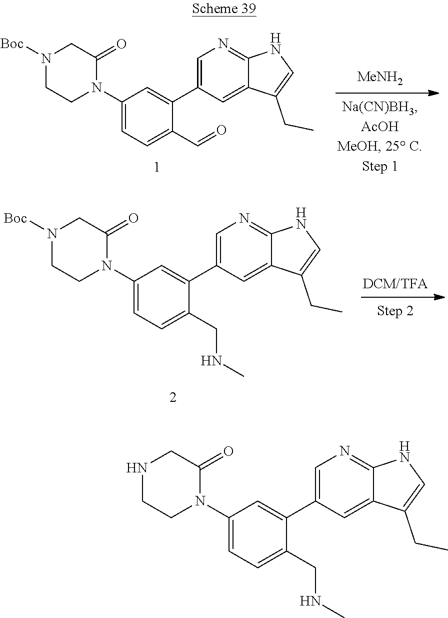 [SMILES: "MeNH2" ; CCc1c[nH]c2ncc(-c3cc(N4CCN(C(=O)OC(C)(C)C)CC4=O)ccc3C=O)cc12 ; CCc1c[nH]c2ncc(-c3cc(N4CCN(C(=O)OC(C)(C)C)CC4=O)ccc3CNC)cc12 ; CCc1c[nH]c2ncc(-c3cc(N4CCNCC4=O)ccc3CNC)cc12]

### Step 1. Preparation of tert-butyl 4-(3-(3-ethyl-1H-pyrrolo[2,3-b]pyridin-5-yl)-4-((methylamino)methyl)phenyl)-3-oxopiperazine-1-carboxylate

Following General Step E, tert-butyl 4-(3-(3-ethyl-1H-pyrrolo[2,3-b]pyridin-5-yl)-4-((methylamino)methyl)phenyl)-3-oxopiperazine-1-carboxylate was prepared as a light yellow solid (120 mg, 52%). Mass (m/z): 464.3 [M+H]⁺.

### Step 2. Preparation of 1-(3-(3-ethyl-1H-pyrrolo[2,3-b]pyridin-5-yl)-4-((methylamino)methyl)phenyl)piperazin-2-one

Following General Step B2, 1-(3-(3-ethyl-1H-pyrrolo[2,3-b]pyridin-5-yl)-4-((methylamino)methyl)phenyl)piperazin-2-one was prepared as a light yellow solid (2.6 mg, 2.7%). Mass (m/z): 364.3 [M+H]⁺.

### Example 42

### 4-(L-valyl)-1-(3-(4-chloro-3-ethyl-1H-pyrrolo[2,3-b]pyridin-5-yl)phenyl)piperazin-2-one

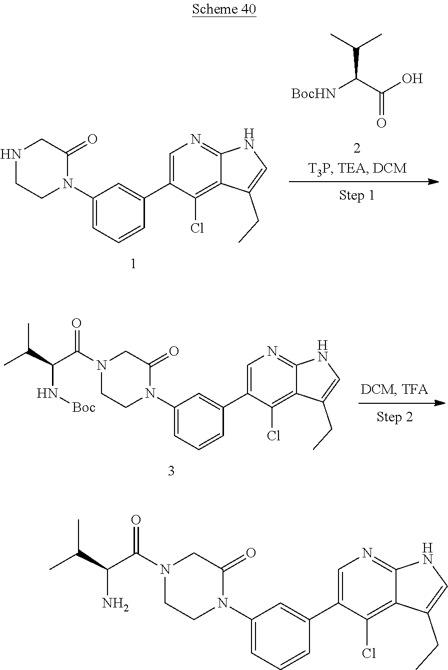 [SMILES: CC(C)[C@H](NC(=O)OC(C)(C)C)C(=O)O ; CCc1c[nH]c2ncc(-c3cccc(N4CCNCC4=O)c3)c(Cl)c12 ; "2T3P, TEA, DCM" ; CCc1c[nH]c2ncc(-c3cccc(N4CCN(C(=O)[C@@H](NC(=O)OC(C)(C)C)C(C)C)CC4=O)c3)c(Cl)c12 ; CCc1c[nH]c2ncc(-c3cccc(N4CCN(C(=O)[C@@H](N)C(C)C)CC4=O)c3)c(Cl)c12]

### Step 1. tert-butyl (S)-(1-(4-(3-(4-chloro-3-ethyl-1H-pyrrolo[2,3-b]pyridin-5-yl)phenyl)-3-oxopiperazin-1-yl)-3-methyl-1-oxobutan-2-yl)carbamate

Following General Step C, tert-butyl (S)-(1-(4-(3-(4-chloro-3-ethyl-1H-pyrrolo[2,3-b]pyridin-5-yl)phenyl)-3-oxopiperazin-1-yl)-3-methyl-1-oxobutan-2-yl)carbamate was prepared as a yellow oil (68 mg, 85%). Mass (m/z): 554.3 [M+H]⁺.

### Step 2. 4-(L-valyl)-1-(3-(4-chloro-3-ethyl-1H-pyrrolo[2,3-b]pyridin-5-yl)phenyl)piperazin-2-one

Following General Step B2, 4-(L-valyl)-1-(3-(4-chloro-3-ethyl-1H-pyrrolo[2,3-b]pyridin-5-yl)phenyl)piperazin-2-one was prepared as a white solid (35 mg, 71%). Mass (m/z): 454.2 [M+H]⁺.

### Example 43

### 4-(L-phenylalanyl)-1-(3-(4-chloro-3-ethyl-1H-pyrrolo[2,3-b]pyridin-5-yl)phenyl)piperazin-2-one

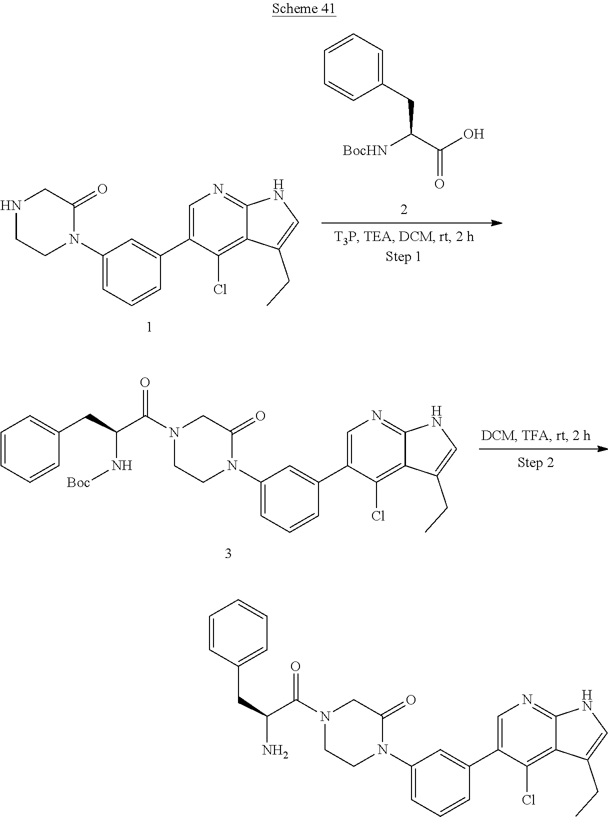

### Step 1. Preparation of tert-butyl (S)-(1-(4-(3-(4-chloro-3-ethyl-1H-pyrrolo[2,3-b]pyridin-5-yl)phenyl)-3-oxopiperazin-1-yl)-1-oxo-3-phenylpropan-2-yl)carbamate

Following General Step C, tert-butyl (S)-(1-(4-(3-(4-chloro-3-ethyl-1H-pyrrolo[2,3-b]pyridin-5-yl)phenyl)-3-oxopiperazin-1-yl)-1-oxo-3-phenylpropan-2-yl)carbamate was prepared as a yellow solid (50 mg, 56%). Mass (m/z): 602.3 [M+H]⁺.

### Step 2. Preparation of 4-(L-phenylalanyl)-1-(3-(4-chloro-3-ethyl-1H-pyrrolo[2,3-b]pyridin-5-yl)phenyl)piperazin-2-one

Following General Step B2, 4-(L-phenylalanyl)-1-(3-(4-chloro-3-ethyl-1H-pyrrolo[2,3-b]pyridin-5-yl)phenyl)piperazin-2-one was prepared as a yellow solid (37 mg, 85%). Mass (m/z): 502.2 [M+H]⁺.

### Example 44

### (S)-2-amino-N-(2-(4-(3-(4-chloro-3-ethyl-1H-pyrrolo[2,3-b]pyridin-5-yl)phenyl)-3-oxopiperazin-1-yl)-2-oxoethyl)-3-methylbutanamide

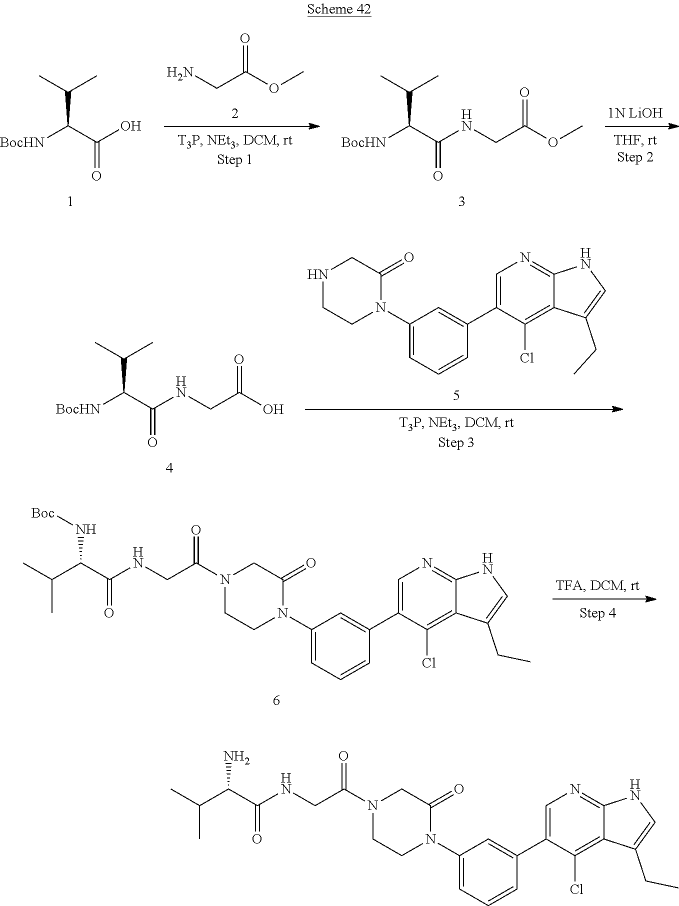

### Step 1. Preparation of methyl (tert-butoxycarbonyl)-L-valylglycinate

Following General Step C, methyl (tert-butoxycarbonyl)-L-valylglycinate was prepared as a white solid (800 mg, 99%). Mass (m/z): 233.1 [M+H−56]⁺.

### Step 2. Preparation of (tert-butoxycarbonyl)-L-valylglycine

Following General Step N, (tert-butoxycarbonyl)-L-valylglycine was prepared as a colorless oil (700 mg, 81%). Mass (m/z): 547.4 [2M−H]⁻.

### Step 3. Preparation of tert-butyl (S)-(1-((2-(4-(3-(4-chloro-3-ethyl-1H-pyrrolo[2,3-b]pyridin-5-yl)phenyl)-3-oxopiperazin-1-yl)-2-oxoethyl)amino)-3-methyl-1-oxobutan-2-yl)carbamate

Following General Step C, tert-butyl (S)-(1-((2-(4-(3-(4-chloro-3-ethyl-1H-pyrrolo[2,3-b]pyridin-5-yl)phenyl)-3-oxopiperazin-1-yl)-2-oxoethyl)amino)-3-methyl-1-oxobutan-2-yl)carbamate was prepared as a yellow solid (36 mg, 32%). Mass (m/z): 611.4 [M+H]⁺.

### Step 4. Preparation of (S)-2-amino-N-(2-(4-(3-(4-chloro-3-ethyl-1H-pyrrolo[2,3-b]pyridin-5-yl)phenyl)-3-oxopiperazin-1-yl)-2-oxoethyl)-3-methylbutanamide

Following General Step B2, (S)-2-amino-N-(2-(4-(3-(4-chloro-3-ethyl-1H-pyrrolo[2,3-b]pyridin-5-yl)phenyl)-3-oxopiperazin-1-yl)-2-oxoethyl)-3-methylbutanamide was prepared as a white solid (6.5 mg, 25%). Mass (m/z): 511.2 [M+H]⁺.

### Example 45

### (R)-2-amino-3-(4-(2-(4-(3-(4-chloro-3-ethyl-1H-pyrrolo[2,3-b]pyridin-5-yl)phenyl)-3-oxopiperazin-1-yl)-2-oxoethoxy)phenyl)propanoic acid

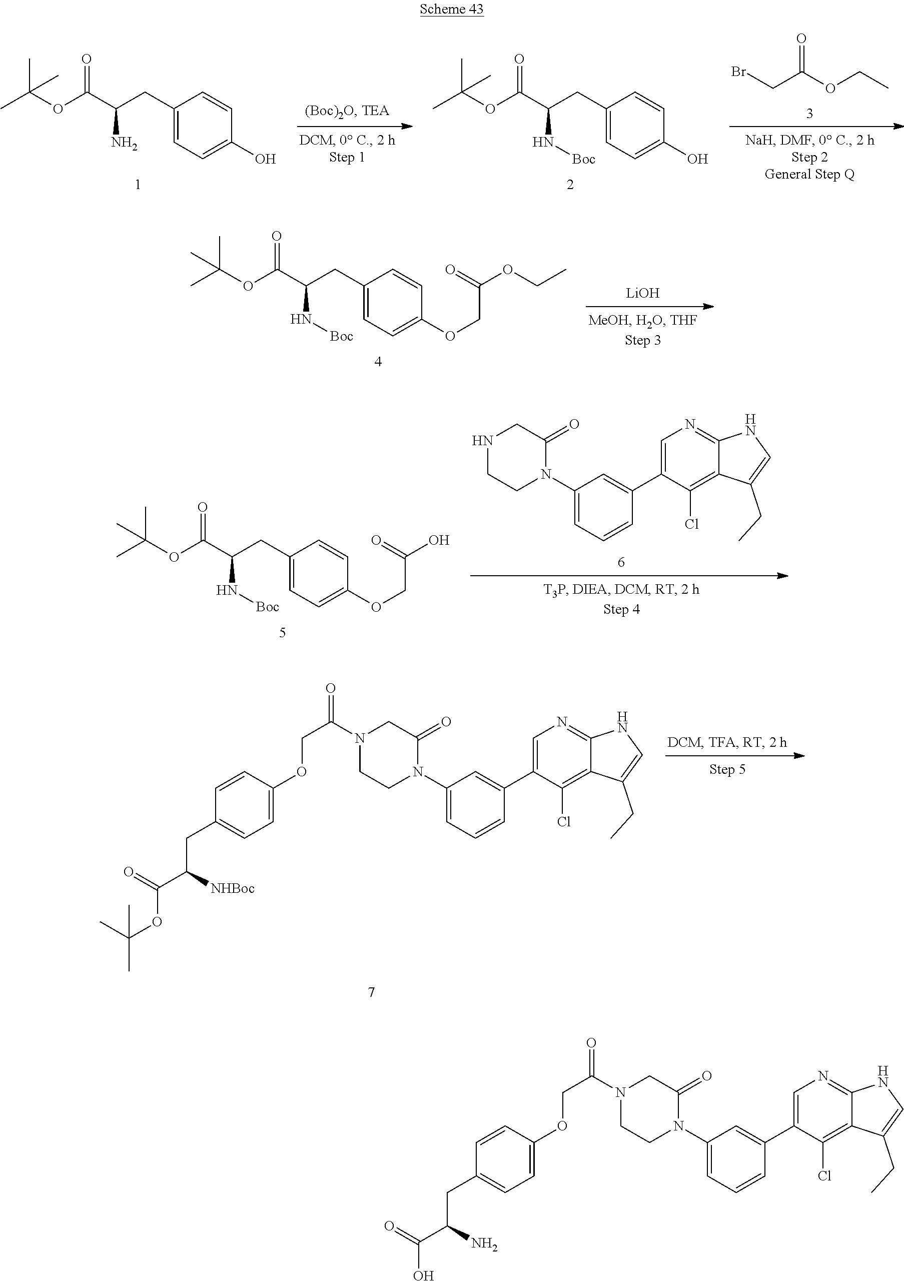

### Step 1. Preparation of tert-butyl (tert-butoxycarbonyl)-D-tyrosinate

Following General Step J, tert-butyl (tert-butoxycarbonyl)-D-tyrosinate was prepared as a yellow solid (1 g, 71%). Mass (m/z): 338.2 [M+H]⁺.

### Step 2. (General Step Q) Preparation of tert-butyl (R)-2-((tert-butoxycarbonyl)amino)-3-(4-(2-ethoxy-2-oxoethoxy)phenyl)propanoate

To a solution of tert-butyl (tert-butoxycarbonyl)-D-tyrosinate (1.0 g, 3.0 mmol) in DMF (10 mL) were sequentially added NaH (60% in oil, 144 mg, 3.6 mmol) and ethyl 2-bromoacetate (500 mg, 3.0 mmol) at 0° C. The resulting mixture was stirred at 0° C. under N₂ for 2 hours, and then diluted with H₂O (20 mL). The aqueous solution was extracted with EA (30 mL×3). The combined organic layers were washed with brine (20 mL×3), dried over with anhydrous Na₂SO₄, and then filtered. The filtrate was concentrated under vacuum. The crude residue was purified by Combiflash (PE/EA=0-12%) to give the product tert-butyl (R)-2-((tert-butoxycarbonyl)amino)-3-(4-(2-ethoxy-2-oxoethoxy)phenyl)propanoate as a yellow solid (758 mg, 60%). Mass (m/z): 424.2 [M+H]⁺.

### Step 3. Preparation of (R)-2-(4-(3-(tert-butoxy)-2-((tert-butoxycarbonyl)amino)-3-oxopropyl)phenoxy)acetic acid

Following General Step N, (R)-2-(4-(3-(tert-butoxy)-2-((tert-butoxycarbonyl)amino)-3-oxopropyl)phenoxy)acetic acid was prepared as a yellow solid (20 mg, 66%). Mass (m/z): 396.2 [M+H]⁺.

### Step 4. Preparation of tert-butyl (R)-2-((tert-butoxycarbonyl)amino)-3-(4-(2-(4-(3-(4-chloro-3-ethyl-1H-pyrrolo[2,3-b]pyridin-5-yl)phenyl)-3-oxopiperazin-1-yl)-2-oxoethoxy)phenyl)propanoate

Following General Step C, tert-butyl (R)-2-((tert-butoxycarbonyl)amino)-3-(4-(2-(4-(3-(4-chloro-3-ethyl-1H-pyrrolo[2,3-b]pyridin-5-yl)phenyl)-3-oxopiperazin-1-yl)-2-oxoethoxy)phenyl)propanoate was prepared as a yellow solid (20 mg, 50%). Mass (m/z): 732.4 [M+H]⁺.

### Step 5. Preparation of (R)-2-amino-3-(4-(2-(4-(3-(4-chloro-3-ethyl-1H-pyrrolo[2,3-b]pyridin-5-yl)phenyl)-3-oxopiperazin-1-yl)-2-oxoethoxy)phenyl)propanoic acid

Following General Step B2, (R)-2-amino-3-(4-(2-(4-(3-(4-chloro-3-ethyl-1H-pyrrolo[2,3-b]pyridin-5-yl)phenyl)-3-oxopiperazin-1-yl)-2-oxoethoxy)phenyl)propanoic acid was prepared as a yellow solid (6 mg, 36%). Mass (m/z): 576.3 [M+H]⁺.

### Example 46

### (R)-2-amino-3-(4-((2-(4-(3-(4-chloro-3-ethyl-1H-pyrrolo[2,3-b]pyridin-5-yl)phenyl)-3-oxopiperazin-1-yl)-2-oxoethyl)amino)phenyl)propanoic acid

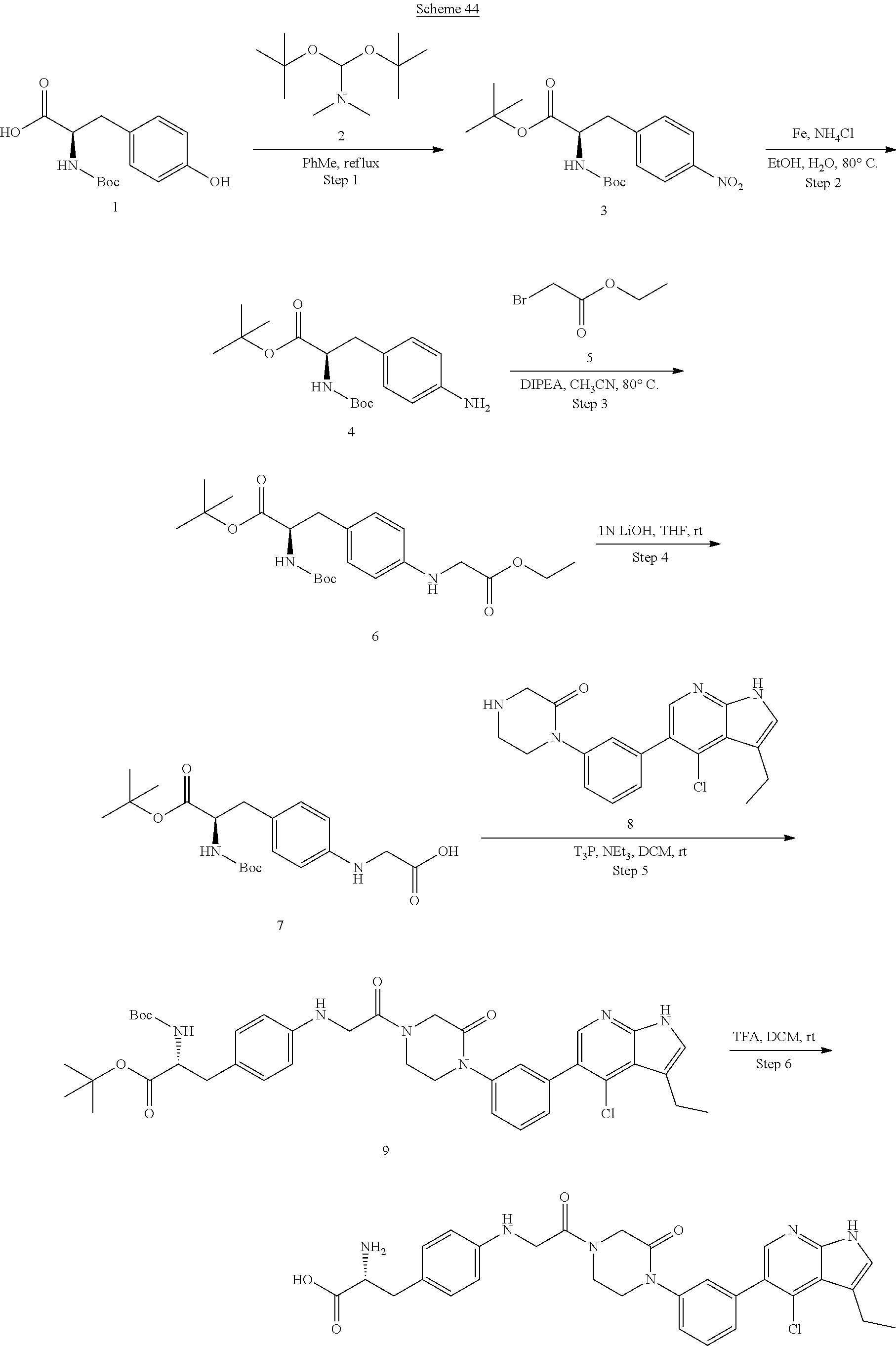

### Step 1. Preparation of tert-butyl (R)-2-((tert-butoxycarbonyl)amino)-3-(4-nitrophenyl) propanoate

To a solution of (R)-2-((tert-butoxycarbonyl)amino)-3-(4-nitrophenyl)propanoic acid (1.56 g, 5.01 mmol) in toluene (20 mL) was added 1,1-di-tert-butoxy-N,N-di methylmethanamine (1.52 g, 7.48 mmol). The reaction mixture was stirred at 110° C. under N₂ for 8 hrs. After the reaction completed, the solvent was removed under reduced pressure and the residue was purified by Combiflash (PE/EA=0˜10%) to give the product tert-butyl (R)-2-((tert-butoxycarbonyl)amino)-3-(4-nitrophenyl) propanoate as a yellow solid (0.56 g, 30%).

### Step 2. Preparation of tert-butyl (R)-3-(4-aminophenyl)-2-((tert-butoxycarbonyl) amino)propanoate

To a solution of tert-butyl (R)-2-((tert-butoxycarbonyl)amino)-3-(4-nitrophenyl)propanoate (560 mg, 1.52 mmol) in EtOH (16 mL) and H₂O (4 mL) were added Fe (514 mg, 9.15 mmol) and NH₄Cl (1.62 g, 30.48 mmol). The reaction mixture was stirred at 80° C. under N₂ for 0.5 hr. The solvent was removed under reduced pressure and the residue was purified by Combiflash (DCM/MeOH=0˜10%) to give the product tert-butyl (R)-3-(4-aminophenyl)-2-((tert-butoxycarbonyl)amino)propanoate as a brown oil (477 mg, 92%). Mass (m/z): 237.1 [M+H−100]⁺.

### Step 3. Preparation of tert-butyl (R)-2-((tert-butoxycarbonyl)amino)-3-(4-((2-ethoxy-2-oxoethyl)amino)phenyl)propanoate

To a solution of tert-butyl (R)-3-(4-aminophenyl)-2-((tert-butoxycarbonyl) amino)propanoate (477 g, 1.41 mmol) in CH₃CN (10 mL) were added ethyl 2-bromoacetate (260 mg, 1.55 mmol) and DIPEA (548 mg, 4.24 mmol). The reaction mixture was stirred at 60° C. under N₂ for 6 hrs. The solvent was removed under reduced pressure and the residue was purified by Combiflash (PE/EA=0˜30%) to give the product tert-butyl (R)-2-((tert-butoxycarbonyl)amino)-3-(4-((2-ethoxy-2-oxoethyl) amino)phenyl)propanoate as brown oil (467 mg, 80%). Mass (m/z): 845.5 [2M+H]⁺.

### Step 4. Preparation of (R)-(4-(3-(tert-butoxy)-2-((tert-butoxycarbonyl)amino)-3-oxopropyl)phenyl)glycine

Following General Step N, (R)-(4-(3-(tert-butoxy)-2-((tert-butoxycarbonyl)amino)-3-oxopropyl)phenyl)glycine was prepared as a brown oil (379 mg, 88%). Mass (m/z): 787.4 [2M−H]⁻.

### Step 5. Preparation of tert-butyl (R)-2-((tert-butoxycarbonyl)amino)-3-(4-((2-(4-(3-(4-chloro-3-ethyl-1H-pyrrolo[2,3-b]pyridin-5-yl)phenyl)-3-oxopiperazin-1-yl)-2-oxoethyl)amino)phenyl)propanoate

Following General Step C, tert-butyl (R)-2-((tert-butoxycarbonyl)amino)-3-(4-((2-(4-(3-(4-chloro-3-ethyl-1H-pyrrolo[2,3-b]pyridin-5-yl)phenyl)-3-oxopiperazin-1-yl)-2-oxoethyl)amino)phenyl)propanoate was prepared as a yellow oil (100 mg, 47%). Mass (m/z): 731.4 [M+H]⁺.

### Step 6. Preparation of (R)-2-amino-3-(4-((2-(4-(3-(4-chloro-3-ethyl-1H-pyrrolo[2,3-b]pyridin-5-yl)phenyl)-3-oxopiperazin-1-yl)-2-oxoethyl)amino)phenyl)propanoic acid

Following General Step B2, (R)-2-amino-3-(4-((2-(4-(3-(4-chloro-3-ethyl-1H-pyrrolo[2,3-b]pyridin-5-yl)phenyl)-3-oxopiperazin-1-yl)-2-oxoethyl)amino)phenyl)propanoic acid was prepared as a white solid (5.2 mg, 6.6%). Mass (m/z): 575.3 [M+H]⁺.

### Example 47

### (S)-2-(4-(((2-amino-4-oxo-3,4-dihydropteridin-6-yl)methyl)amino)benzamido)-5-(4-(3-(4-chloro-3-ethyl-1H-pyrrolo[2,3-b]pyridin-5-yl)phenyl)-3-oxopiperazin-1-yl)-5-oxopentanoic acid

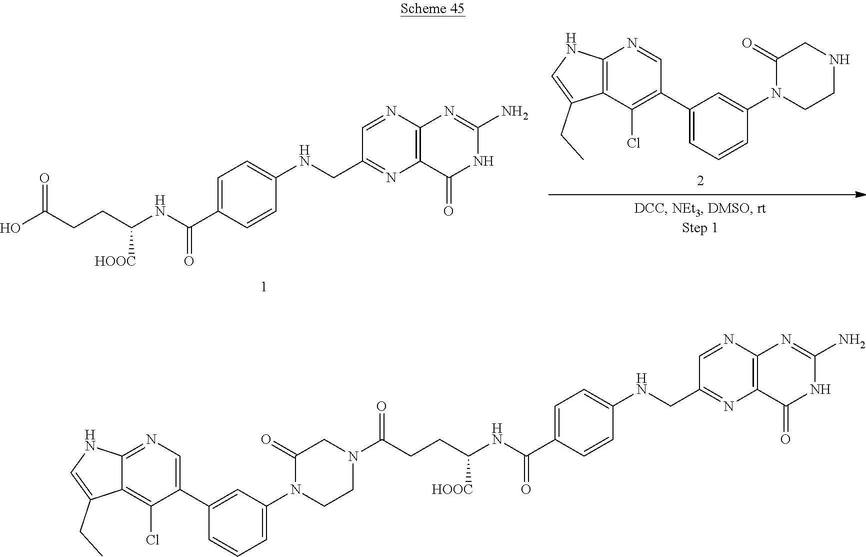 [SMILES: Nc1nc2ncc(CNc3ccc(C(=O)N[C@@H](CCC(=O)O)C(=O)O)cc3)nc2c(=O)[nH]1 ; CCc1c[nH]c2ncc(-c3cccc(N4CCNCC4=O)c3)c(Cl)c12 ; CCc1c[nH]c2ncc(-c3cccc(N4CCN(C(=O)CC[C@H](NC(=O)c5ccc(NCc6cnc7nc(N)[nH]c(=O)c7n6)cc5)C(=O)O)CC4=O)c3)c(Cl)c12]

### Step 1. Preparation of (S)-2-(4-(((2-amino-4-oxo-3,4-dihydropteridin-6-yl)methyl)amino)benzamido)-5-(4-(3-(4-chloro-3-ethyl-1H-pyrrolo[2,3-b]pyridin-5-yl)phenyl)-3-oxopiperazin-1-yl)-5-oxopentanoic acid

Following General Step C, (S)-2-(4-(((2-amino-4-oxo-3,4-dihydropteridin-6-yl)methyl)amino)benzamido)-5-(4-(3-(4-chloro-3-ethyl-1H-pyrrolo[2,3-b]pyridin-5-yl)phenyl)-3-oxopiperazin-1-yl)-5-oxopentanoic acid was prepared as a white solid (5 mg, 3.1%). Mass (m/z): 777.8 [M+H]⁺.

### Example 48

### 1-(3-(4-chloro-3-ethyl-1H-pyrrolo[2,3-b]pyridin-5-yl)phenyl)-4-(4-(((2R,3R,4S,5S,6R)-3,4,5-trihydroxy-6-(hydroxymethyl)tetrahydro-2H-pyran-2-yl)oxy)butanoyl)piperazin-2-one

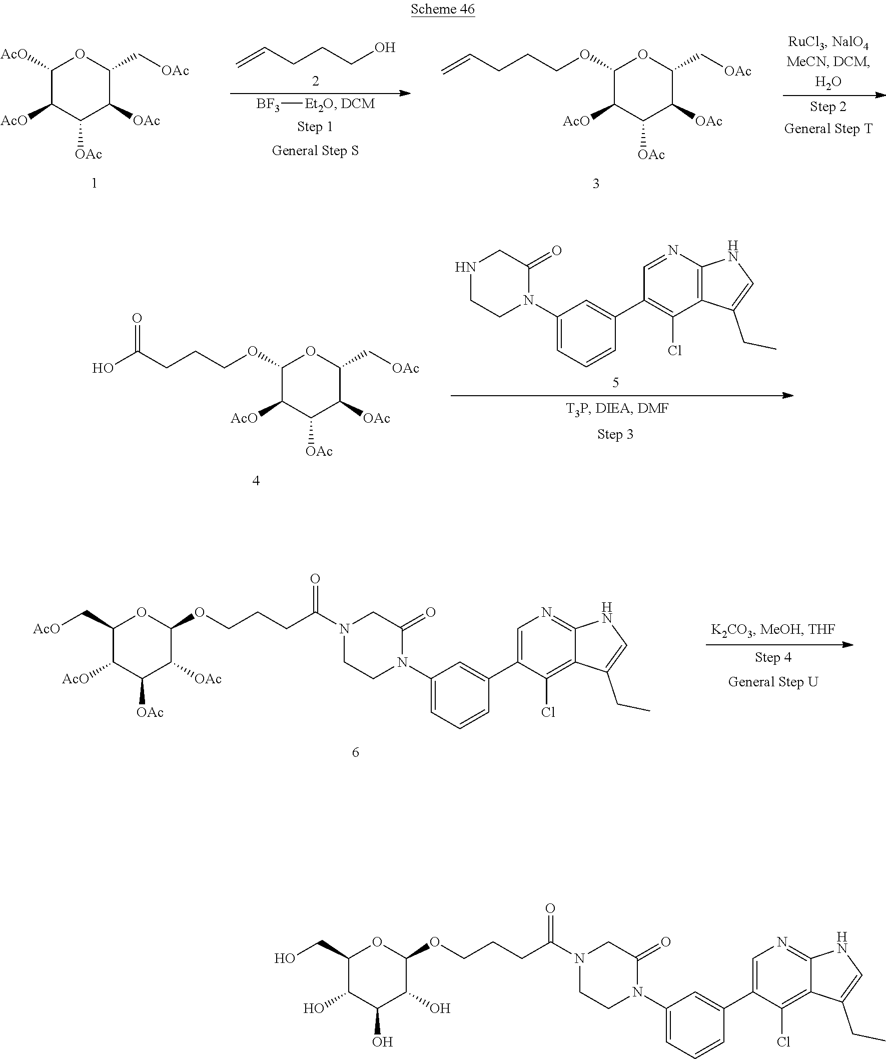

### Step 1. (General Step S) Preparation of (2R,3R,4S,5R,6R)-2-(acetoxymethyl)-6-(pent-4-en-1-yloxy)tetrahydro-2H-pyran-3,4,5-triyl triacetate

To a solution of [(2R,3R,4S,5R,6S)-3,4,5,6-tetrakis(acetyloxy)oxan-2-yl]methyl acetate (5.00 g, 12.8 mmol) in DCM (50 mL) was added boron trifluoride etherate (2.91 g, 20.5 mmol) at 0° C. The resulting solution was stirred at 0° C. for 0.5 hour before the addition of pent-4-en-1-ol (1.43 g, 16.7 mmol) at 0° C. The mixture was further stirred at 25° C. for 16 hours, and then diluted with saturated NaHCO₃ aqueous solution (25 mL). The aqueous media was extracted with DCM (50 mL×2). The combined organic layers were washed with brine (50 mL), dried over Na₂SO₄, and then filtered. The filtrate was concentrated under vacuum. The crude residue was purified by silica gel column chromatography (PE/EA=3:1) to give the product (2R,3R,4S,5R,6R)-2-(acetoxymethyl)-6-(pent-4-en-1-yloxy)tetrahydro-2H-pyran-3,4,5-triyl triacetate as a colorless oil (1.50 g, 28%). Mass (m/z): 438.8 [M+Na]⁺.

### Step 2. (General Step T) Preparation of 4-(((2R,3R,4S,5R,6R)-3,4,5-triacetoxy-6-(acetoxymethyl)tetrahydro-2H-pyran-2-yl)oxy)butanoic acid

To a solution of [(2R,3R,4S,5R,6R)-3,4,5-tris(acetyloxy)-6-(pent-4-en-1-yloxy)oxan-2-yl]methyl acetate (1.36 g, 3.25 mmol) in the mixed solvent of DCM (5.2 mL), MeCN (5.2 mL) and H₂O (7.8 mL) were added NaIO₄ (4.64 g, 13.0 mmol) and RuCl₃-3H₂O (15 mg, 0.065 mmol) at 0° C. The reaction mixture was stirred at 25° C. for 2 hours before the addition of another batch of NaIO₄ (4.64 g, 13.0 mmol). The resulting mixture was further stirred at 25° C. for 2 hours, and then diluted with water (20 mL). The aqueous solution was extracted with DCM (20 mL×3). The combined organic layers were washed with brine (20 mL), dried over Na₂SO₄, and then filtered. The filtrate was concentrated under vacuum. The crude residue was purified by silica gel column chromatography (PE/EA/MeOH=1:1:0.1) to give the product 4-(((2R,3R,4S,5R,6R)-3,4,5-triacetoxy-6-(acetoxymethyl)tetrahydro-2H-pyran-2-yl)oxy)butanoic acid as a colorless oil (1.00 g, 71%). Mass (m/z): 457.1 [M+Na]⁺.

### Step 3. Preparation of (2R,3R,4S,5R,6R)-2-(acetoxymethyl)-6-(4-(4-(3-(4-chloro-3-ethyl-1H-pyrrolo[2,3-b]pyridin-5-yl)phenyl)-3-oxopiperazin-1-yl)-4-oxobutoxy)tetrahydro-2H-pyran-3,4,5-triyl triacetate

Following General Step C, (2R,3R,4S,5R,6R)-2-(acetoxymethyl)-6-(4-(4-(3-(4-chloro-3-ethyl-1H-pyrrolo[2,3-b]pyridin-5-yl)phenyl)-3-oxopiperazin-1-yl)-4-oxobutoxy)tetrahydro-2H-pyran-3,4,5-triyl triacetate was prepared as a colorless oil (380 mg, 76%). Mass (m/z): 770.7 [M+H]⁺.

### Step 4. (General Step U) Preparation of 1-(3-(4-chloro-3-ethyl-1H-pyrrolo[2,3-b]pyridin-5-yl)phenyl)-4-(4-(((2R,3R,4S,5S,6R)-3,4,5-trihydroxy-6-(hydroxymethyl)tetrahydro-2H-pyran-2-yl)oxy)butanoyl)piperazin-2-one

To a solution of (2R,3R,4S,5R,6R)-2-(acetoxymethyl)-6-(4-(4-(3-(4-chloro-3-ethyl-1H-pyrrolo[2,3-b]pyridin-5-yl)phenyl)-3-oxopiperazin-1-yl)-4-oxobutoxy)tetrahydro-2H-pyran-3,4,5-triyl triacetate (380 mg, 0.49 mmol) in the mixed solvent of THF (3 mL) and MeOH (3 mL) was added K₂CO₃ (136 mg, 0.99 mmol). The resulting mixture was stirred at 25° C. under N₂ for 2 hours, and then concentrated under vacuum. The crude residue was purified by silica gel column chromatography (DCM/MeOH=20:1) to give the product 1-(3-(4-chloro-3-ethyl-1H-pyrrolo[2,3-b]pyridin-5-yl)phenyl)-4-(4-(((2R,3R,4S,5S,6R)-3,4,5-trihydroxy-6-(hydroxymethyl)tetrahydro-2H-pyran-2-yl)oxy)butanoyl)piperazin-2-one as a white solid (119 mg, 40%). Mass (m/z): 602.8 [M+H]⁺.

### Example 49

### 1-(3-(4-chloro-3-ethyl-1H-pyrrolo[2,3-b]pyridin-5-yl)phenyl)-4-(2-(((2R,3R,4S,5S,6R)-3,4,5-trihydroxy-6-(hydroxymethyl)tetrahydro-2H-pyran-2-yl)oxy)acetyl)piperazin-2-one

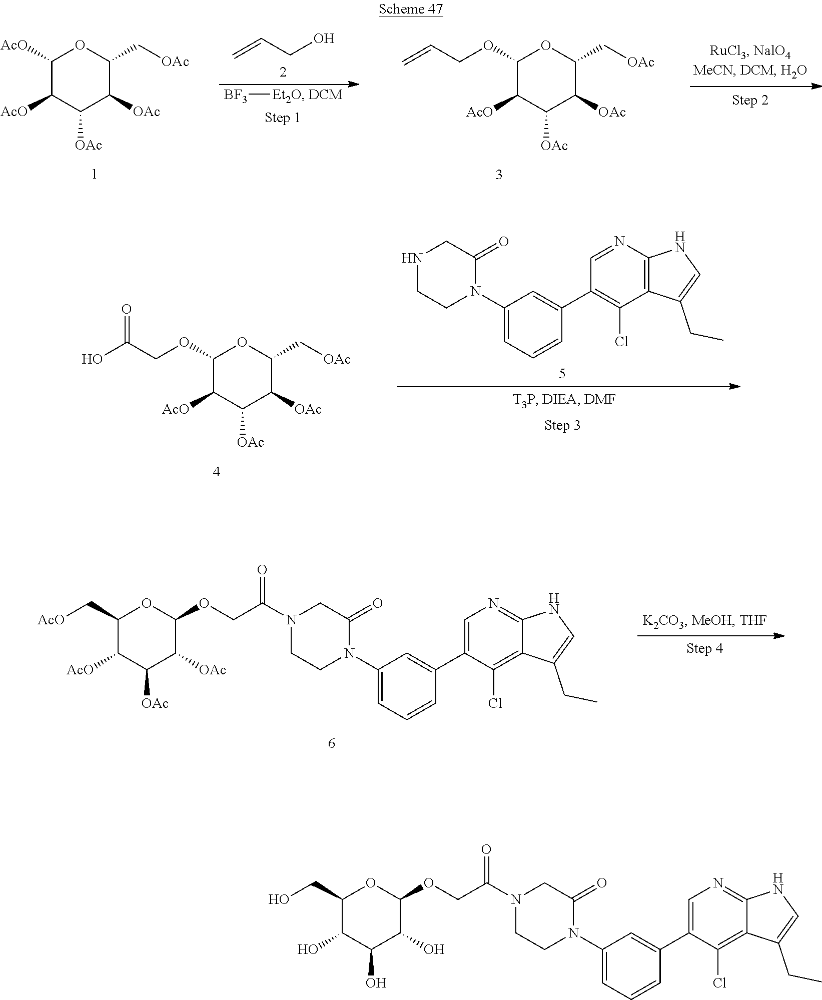

### Step 1. Preparation of (2R,3R,4S,5R,6R)-2-(acetoxymethyl)-6-(allyloxy)tetrahydro-2H-pyran-3,4,5-triyl triacetate

Following General Step S, (2R,3R,4S,5R,6R)-2-(acetoxymethyl)-6-(allyloxy)tetrahydro-2H-pyran-3,4,5-triyl triacetate was prepared as a colorless oil (2.80 g, 45%). Mass (m/z): 410.8 [M+Na]⁺.

### Step 2. Preparation of 2-(((2R,3R,4S,5R,6R)-3,4,5-triacetoxy-6-(acetoxymethyl)tetrahydro-2H-pyran-2-yl)oxy)acetic acid

Following General Step T, 2-(((2R,3R,4S,5R,6R)-3,4,5-triacetoxy-6-(acetoxymethyl)tetrahydro-2H-pyran-2-yl)oxy)acetic acid was prepared as a colorless oil (700 mg, 50%). Mass (m/z): 428.8 [M+Na]⁺.

### Step 3. Preparation of (2R,3R,4S,5R,6R)-2-(acetoxymethyl)-6-(2-(4-(3-(4-chloro-3-ethyl-1H-pyrrolo[2,3-b]pyridin-5-yl)phenyl)-3-oxopiperazin-1-yl)-2-oxoethoxy)tetrahydro-2H-pyran-3,4,5-triyl triacetate

Following General Step C, (2R,3R,4S,5R,6R)-2-(acetoxymethyl)-6-(2-(4-(3-(4-chloro-3-ethyl-1H-pyrrolo[2,3-b]pyridin-5-yl)phenyl)-3-oxopiperazin-1-yl)-2-oxoethoxy)tetrahydro-2H-pyran-3,4,5-triyl triacetate was prepared as a colorless oil (120 mg, 71%). Mass (m/z): 742.7 [M+H]⁺.

### Step 4. Preparation of 1-(3-(4-chloro-3-ethyl-1H-pyrrolo[2,3-b]pyridin-5-yl)phenyl)-4-(2-(((2R,3R,4S,5S,6R)-3,4,5-trihydroxy-6-(hydroxymethyl)tetrahydro-2H-pyran-2-yl)oxy)acetyl)piperazin-2-one

Following General Step U, 1-(3-(4-chloro-3-ethyl-1H-pyrrolo[2,3-b]pyridin-5-yl)phenyl)-4-(2-(((2R,3R,4S,5S,6R)-3,4,5-trihydroxy-6-(hydroxymethyl)tetrahydro-2H-pyran-2-yl)oxy)acetyl)piperazin-2-one was prepared as a white solid (22 mg, 21%). Mass (m/z): 574.7 [M+H]⁺.

### Example 50

### 1-(3-(4-chloro-3-ethyl-1H-pyrrolo[2,3-b]pyridin-5-yl)phenyl)-4-(2-(3-((3R,4R,5S,6R)-3,4,5-trihydroxy-6-(hydroxymethyl)tetrahydro-2H-pyran-2-yl)phenoxy)acetyl)piperazin-2-one

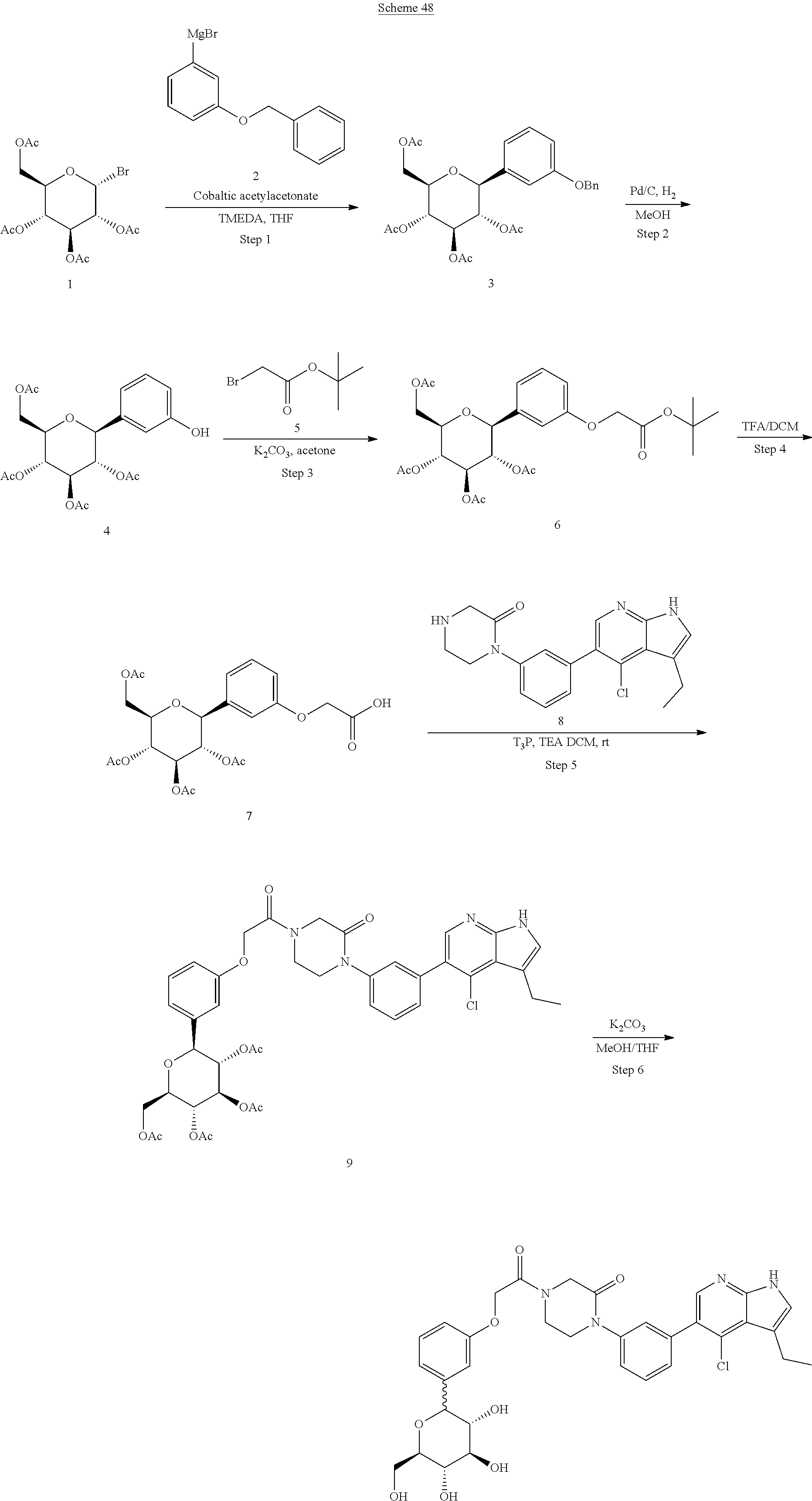

### Step 1. Preparation of (2R,3R,4R,5S,6S)-2-(acetoxymethyl)-6-(3-(benzyloxy)phenyl) tetrahydro-2H-pyran-3,4,5-triyl triacetate

To a solution of (2R,3R,4S,5R,6R)-2-(acetoxymethyl)-6-bromotetrahydro-2H-pyran-3,4,5-triyl triacetate (2 g, 4.86 mmol) in THF (30 mL) was added Cobaltic acetylacetonate (87 mg, 0.24 mmol) and TMEDA (28 mg, 0.24 mmol) under N₂ at 0° C. And then dripped at a very low rate onto a cold suspension (0-5° C.) of the (3-(benzyloxy)phenyl)magnesium bromide (0.5 M, 14.6 mL, 7.3 mmol). The reaction mixture was stirred at 0° C. under N₂ for 1 hr. After the reaction completed, H₂O (100 mL) was added to the reaction mixture, and then extracted with EA (50 mL×3). The combined organic layer was washed with brine (50 mL×3), then dried over with anhydrous Na₂SO₄. After filtration, the solution was concentration under vacuum, and the crude product was purified by Combiflash (PE/EA=0˜50%) to give the product (2R,3R,4R,5S,6S)-2-(acetoxymethyl)-6-(3-(benzyloxy)phenyl)tetrahydro-2H-pyran-3,4,5-triyl triacetate as a yellow solid (2.2 g, 87%). Mass (m/z): 536.7 [M+Na]⁺.

### Step 2. Preparation of (2R,3R,4R,5S,6S)-2-(acetoxymethyl)-6-(3-hydroxyphenyl) tetrahydro-2H-pyran-3,4,5-triyl triacetate

To a solution of (2R,3R,4R,5S,6S)-2-(acetoxymethyl)-6-(3-(benzyloxy)phenyl) tetrahydro-2H-pyran-3,4,5-triyl triacetate (1.1 g, 2.13 mmol) in THF (20 mL) was added 10% Pd/C (220 mg, 20% wt/wt). The reaction mixture was stirred at rt under H₂ in a balloon for 16 hrs. The reaction mixture was filtered, the filtrate was concentrated under vacuum to afford crude product and the crude was purified by Combiflash (PE/EA=0˜30%) to give the product (2R,3R,4R,5S,6S)-2-(acetoxymethyl)-6-(3-hydroxyphenyl)tetrahydro-2H-pyran-3,4,5-triyl triacetate as a white solid (841 mg, 92%). Mass (m/z): 446.7 [M+Na]⁺.

### Step 3. Preparation of (2R,3R,4R,5S,6S)-2-(acetoxymethyl)-6-(3-(2-(tert-butoxy)-2-oxoethoxy)phenyl)tetrahydro-2H-pyran-3,4,5-triyl triacetate

To a solution of (2R,3R,4R,5S,6S)-2-(acetoxymethyl)-6-(3-hydroxyphenyl) tetrahydro-2H-pyran-3,4,5-triyl triacetate (840 mg, 1.97 mmol) in Me₂CO (10 mL) was added tert-butyl 2-bromoacetate (579 mg, 2.96 mmol) and K₂CO₃ (410 mg, 2.96 mmol) The reaction mixture was stirred at rt for 16 hrs. The reaction mixture was filtered, the filtrate was concentrated under vacuum to afford crude product and the crude was purified by Combiflash (PE/EA=0˜30%) to give the product (2R,3R,4R,5S,6S)-2-(acetoxymethyl)-6-(3-(2-(tert-butoxy)-2-oxoethoxy)phenyl)tetrahydro-2H-pyran-3,4,5-triyl triacetate as a yellow oil (690 mg, 64%). Mass (m/z): 560.8 [M+Na]⁺.

### Step 4. Preparation of 2-(3-((2S,3S,4R,5R,6R)-3,4,5-triacetoxy-6-(acetoxymethyl) tetrahydro-2H-pyran-2-yl)phenoxy)acetic acid

Following General Step B2, 2-(3-((2S,3S,4R,5R,6R)-3,4,5-triacetoxy-6-(acetoxymethyl) tetrahydro-2H-pyran-2-yl)phenoxy)acetic acid was prepared as a brown oil (550 mg, 88%). Mass (m/z): 504.8 [M+Na]⁺.

### Step 5. Preparation of (2R,3R,4R,5S,6S)-2-(acetoxymethyl)-6-(3-(2-(4-(3-(4-chloro-3-ethyl-1H-pyrrolo[2,3-b]pyridin-5-yl)phenyl)-3-oxopiperazin-1-yl)-2oxoethoxy) phenyl)tetrahydro-2H-pyran-3,4,5-triyl triacetate

Following General Step C, (2R,3R,4R,5S,6S)-2-(acetoxymethyl)-6-(3-(2-(4-(3-(4-chloro-3-ethyl-1H-pyrrolo[2,3-b]pyridin-5-yl)phenyl)-3-oxopiperazin-1-yl)-2oxoethoxy) phenyl)tetrahydro-2H-pyran-3,4,5-triyl triacetate was prepared as a yellow solid (840 mg, 90%). Mass (m/z): 818.7 [M+H]⁺.

### Step 6. Preparation of 1-(3-(4-chloro-3-ethyl-1H-pyrrolo[2,3-b]pyridin-5-yl)phenyl)-4-(2-(3-((3R,4R,5S,6R)-3,4,5-trihydroxy-6-(hydroxymethyl)tetrahydro-2H-pyran-2-yl)phenoxy)acetyl)piperazin-2-one

Following General Step U, 1-(3-(4-chloro-3-ethyl-1H-pyrrolo[2,3-b]pyridin-5-yl)phenyl)-4-(2-(3-((3R,4R,5S,6R)-3,4,5-trihydroxy-6-(hydroxymethyl)tetrahydro-2H-pyran-2-yl)phenoxy)acetyl)piperazin-2-one was prepared as a white solid (345 mg, 51%). Mass (m/z): 650.6 [M+H]⁺.

### Example 51

### 1-(3-(4-chloro-3-(2,2-difluoroethyl)-1H-pyrrolo[2,3-b]pyridin-5-yl)phenyl)-4-(methylsulfonyl)piperazin-2-one

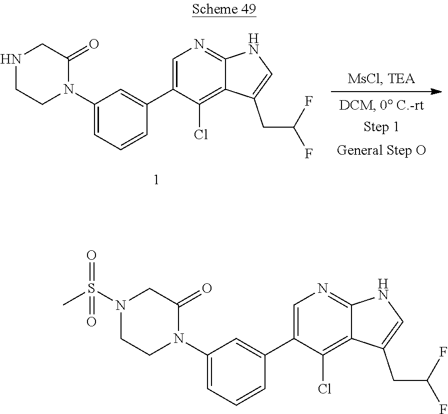 [SMILES: O=C1CNCCN1c1cccc(-c2cnc3[nH]cc(CC(F)F)c3c2Cl)c1 ; CS(=O)(=O)N1CCN(c2cccc(-c3cnc4[nH]cc(CC(F)F)c4c3Cl)c2)C(=O)C1]

### Step 1. (General Step O) Preparation of 1-(3-(4-chloro-3-(2,2-difluoroethyl)-1H-pyrrolo[2,3-b]pyridin-5-yl)phenyl)-4-(methylsulfonyl)piperazin-2-one

To a solution of 1-(3-(4-chloro-3-(2,2-difluoroethyl)-1H-pyrrolo[2,3-b]pyridin-5-yl)phenyl)piperazin-2-one (70 mg, 0.18 mmol) and triethylamine (36 mg, 0.36 mmol) in DCM (5 mL) was added MsCl (23 mg, 0.20 mmol) at 0° C. The resulting solution was stirred at ambient temperature under N₂ for 16 hrs, and then concentrated under reduced pressure. The crude residue was purified by Combiflash (MeOH/DCM=0-2%) to give 1-(3-(4-chloro-3-(2,2-difluoroethyl)-1H-pyrrolo[2,3-b]pyridin-5-yl)phenyl)-4-(methylsulfonyl)piperazin-2-one as a white solid (10 mg, 11%). Mass (m/z): 468.9 [M+H]⁺.

### Example 52

### 1-(3-(4-chloro-3-ethyl-1H-pyrrolo[2,3-b]pyridin-5-yl)phenyl)-4-(methylsulfonyl) piperazin-2-one

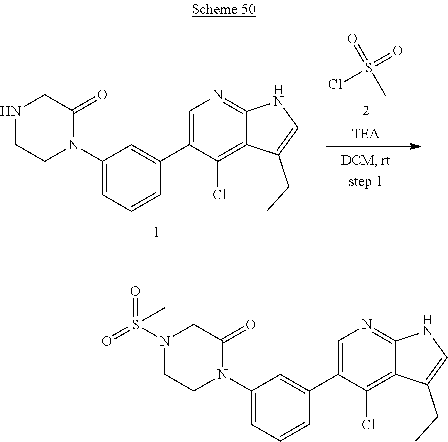

### Step 1. Preparation of 1-(3-(4-chloro-3-ethyl-1H-pyrrolo[2,3-b]pyridin-5-yl)phenyl)-4-(methylsulfonyl)piperazin-2-one

Following General Step O, 1-(3-(4-chloro-3-ethyl-1H-pyrrolo[2,3-b]pyridin-5-yl)phenyl)-4-(methylsulfonyl)piperazin-2-one was prepared as a yellow solid (80 mg, 43%). Mass (m/z): 432.8 [M+H]⁺.

### Example 53

### 1-(3-(4-chloro-3-cyclopropyl-1H-pyrrolo[2,3-b]pyridin-5-yl)phenyl)-4-(methylsulfonyl)piperazin-2-one

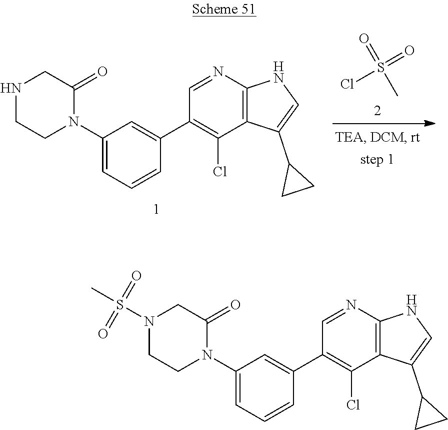

### Step 1. Preparation of 1-(3-(4-chloro-3-cyclopropyl-1H-pyrrolo[2,3-b]pyridin-5-yl) phenyl)-4-(methylsulfonyl)piperazin-2-one

Following General Step O, 1-(3-(4-chloro-3-cyclopropyl-1H-pyrrolo[2,3-b]pyridin-5-yl) phenyl)-4-(methylsulfonyl)piperazin-2-one was prepared as a white solid (16 mg, 6%). Mass (m/z): 444.8 [M+H]⁺.

### Example 54

### 1-(3-(4-chloro-3-cyclopropyl-1H-pyrrolo[2,3-b]pyridin-5-yl)phenyl)-4-((2-methoxyethyl)sulfonyl)piperazin-2-one

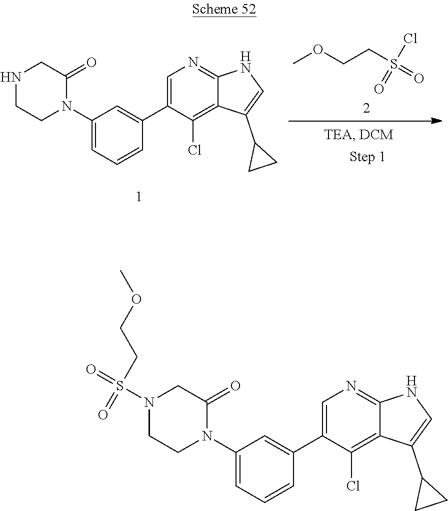

### Step 1. Preparation of 1-(3-(4-chloro-3-cyclopropyl-1H-pyrrolo[2,3-b]pyridin-5-yl)phenyl)-4-((2-methoxyethyl)sulfonyl)piperazin-2-one

Following General Step O, 1-(3-(4-chloro-3-cyclopropyl-1H-pyrrolo[2,3-b]pyridin-5-yl)phenyl)-4-((2-methoxyethyl)sulfonyl)piperazin-2-one was prepared as a brown solid (25 mg, 9%). Mass (m/z): 488.8 [M+H]⁺.

### Example 55

### 1-(3-(4-chloro-3-cyclopropyl-1H-pyrrolo[2,3-b]pyridin-5-yl)phenyl)-4-((3-morpholinopropyl)sulfonyl)piperazin-2-one

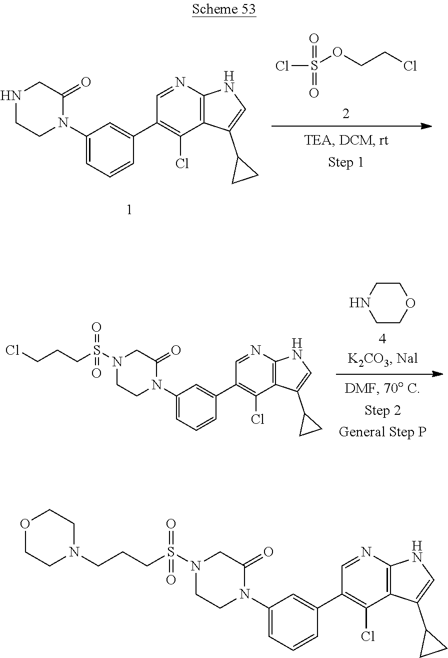 [SMILES: O=C1CNCCN1c1cccc(-c2cnc3[nH]cc(C4CC4)c3c2Cl)c1 ; O=S(=O)(Cl)OCCCl ; C1COCCN1 ; O=C1CN(S(=O)(=O)CCCCl)CCN1c1cccc(-c2cnc3[nH]cc(C4CC4)c3c2Cl)c1 ; O=C1CN(S(=O)(=O)CCCN2CCOCC2)CCN1c1cccc(-c2cnc3[nH]cc(C4CC4)c3c2Cl)c1]

### Step 1. Preparation of 1-(3-(4-chloro-3-cyclopropyl-1H-pyrrolo[2,3-b]pyridin-5-yl)phenyl)-4-((3-chloropropyl)sulfonyl)piperazin-2-one

Following General Step O, 1-(3-(4-chloro-3-cyclopropyl-1H-pyrrolo[2,3-b]pyridin-5-yl)phenyl)-4-((3-chloropropyl)sulfonyl)piperazin-2-one was prepared as a white solid (316 mg, 75%). Mass (m/z): 507.0 [M+H]⁺.

### Step 2. (General Step P) Preparation of 1-(3-(4-chloro-3-cyclopropyl-1H-pyrrolo[2,3-b]pyridin-5-yl)phenyl)-4-((3-morpholinopropyl)sulfonyl)piperazin-2-one

To a solution of 1-(3-(4-chloro-3-cyclopropyl-1H-pyrrolo[2,3-b]pyridin-5-yl)phenyl)-4-((3-chloropropyl)sulfonyl)piperazin-2-one (300 mg, 0.59 mmol) in DMF (10 mL) were added morpholine (77 mg, 0.89 mmol), K₂CO₃ (163 mg, 1.18 mmol) and NaI (177 mg, 1.18 mmol). The resulting mixture was stirred at 70° C. under N₂ for 3 hours, and then diluted with H₂O (50 mL). The aqueous media was extracted with EA (30 mL×3). The combined organic layers were washed with brine (30 mL×3), dried over anhydrous Na₂SO₄, and filtered. The filtrate was concentrated under vacuum. The crude residue was purified by preparative reverse-phase HPLC [Gemini-C18, 150×21.2 mm, 5 um, ACN-H₂O (0.1% TFA), 10-40%] to give 1-(3-(4-chloro-3-cyclopropyl-1H-pyrrolo[2,3-b]pyridin-5-yl)phenyl)-4-((3-morpholinopropyl)sulfonyl)piperazin-2-one as a white solid (75 mg, 22%). Mass (m/z): 558.0 [M+H]⁺.

### Example 56

### 1-(3-(4-chloro-3-cyclopropyl-1H-pyrrolo[2,3-b]pyridin-5-yl)phenyl)-4-((3-(dimethylamino)propyl)sulfonyl)piperazin-2-one

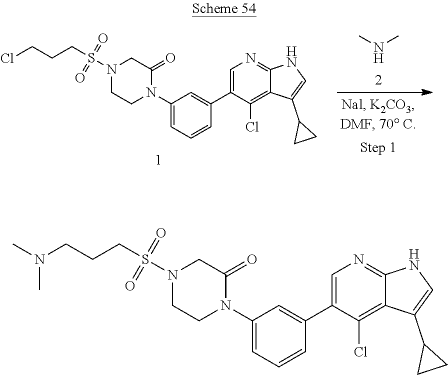

### Step 1. Preparation of 1-(3-(4-chloro-3-cyclopropyl-1H-pyrrolo[2,3-b]pyridin-5-yl)phenyl)-4-((3-(dimethylamino)propyl)sulfonyl)piperazin-2-one

Following General Step P, 1-(3-(4-chloro-3-cyclopropyl-1H-pyrrolo[2,3-b]pyridin-5-yl)phenyl)-4-((3-(dimethylamino)propyl)sulfonyl)piperazin-2-one was prepared as a yellow solid (400 mg, 29%). Mass (m/z): 516.1 [M+H]⁺.

### Example 57

### 1-(3-(4-chloro-3-cyclopropyl-1H-pyrrolo[2,3-b]pyridin-5-yl)phenyl)-4-((3-(3-methylazetidin-1-yl)propyl)sulfonyl)piperazin-2-one

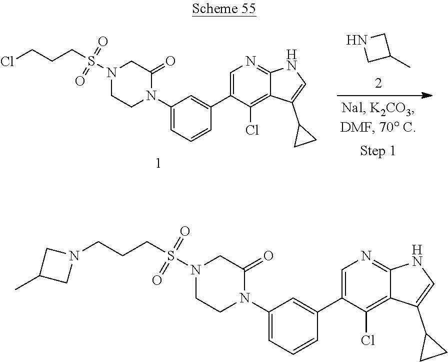

### Step 1. 1-(3-(4-chloro-3-cyclopropyl-1H-pyrrolo[2,3-b]pyridin-5-yl)phenyl)-4-((3-(3-methylazetidin-1-yl)propyl)sulfonyl)piperazin-2-one

Following General Step P, 1-(3-(4-chloro-3-cyclopropyl-1H-pyrrolo[2,3-b]pyridin-5-yl)phenyl)-4-((3-(3-methylazetidin-1-yl)propyl)sulfonyl)piperazin-2-one was prepared as a yellow solid (20 mg, 12%). Mass (m/z): 542.2 [M+H]⁺.

### Example 58

### 1-(3-(4-chloro-3-cyclopropyl-1H-pyrrolo[2,3-b]pyridin-5-yl)phenyl)-4-((3-(3-fluoroazetidin-1-yl)propyl)sulfonyl)piperazin-2-one

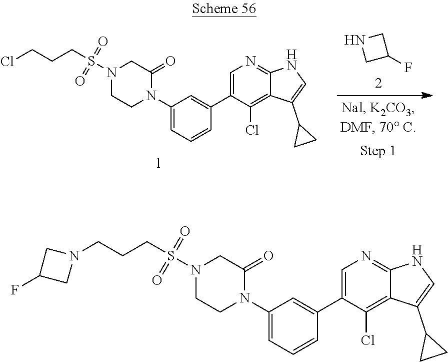 [SMILES: O=C1CN(S(=O)(=O)CCCCl)CCN1c1cccc(-c2cnc3[nH]cc(C4CC4)c3c2Cl)c1 ; FC1CNC1 ; O=C1CN(S(=O)(=O)CCCN2CC(F)C2)CCN1c1cccc(-c2cnc3[nH]cc(C4CC4)c3c2Cl)c1]

### Step 1. 1-(3-(4-chloro-3-cyclopropyl-1H-pyrrolo[2,3-b]pyridin-5-yl)phenyl)-4-((3-(3-fluoroazetidin-1-yl)propyl)sulfonyl)piperazin-2-one

Following General Step P, 1-(3-(4-chloro-3-cyclopropyl-1H-pyrrolo[2,3-b]pyridin-5-yl)phenyl)-4-((3-(3-fluoroazetidin-1-yl)propyl)sulfonyl)piperazin-2-one was prepared as a light yellow solid (15 mg, 9.0%). Mass (m/z): 546.2 [M+H]⁺.

### Example 59

### 1-(3-(4-chloro-3-(2,2-difluoroethyl)-1H-pyrrolo[2,3-b]pyridin-5-yl)phenyl)-4-((3-morpholinopropyl)sulfonyl)piperazin-2-one

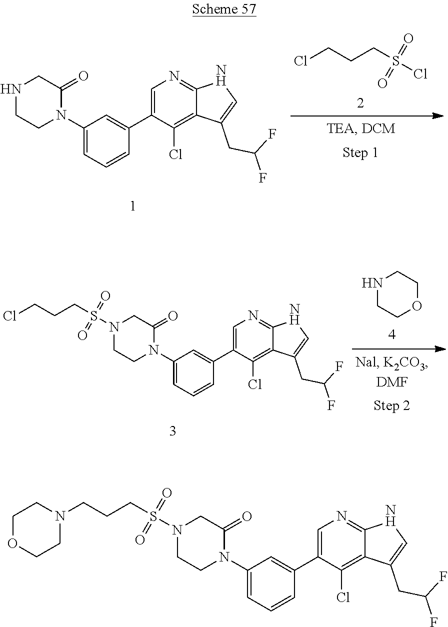 [SMILES: O=C1CNCCN1c1cccc(-c2cnc3[nH]cc(CC(F)F)c3c2Cl)c1 ; O=S(=O)(Cl)CCCCl ; O=C1CN(S(=O)(=O)CCCCl)CCN1c1cccc(-c2cnc3[nH]cc(CC(F)F)c3c2Cl)c1 ; C1COCCN1 ; O=C1CN(S(=O)(=O)CCCN2CCOCC2)CCN1c1cccc(-c2cnc3[nH]cc(CC(F)F)c3c2Cl)c1]

### Step 1. Preparation of 1-(3-(4-chloro-3-(2,2-difluoroethyl)-1H-pyrrolo[2,3-b]pyridin-5-yl)phenyl)-4-((3-chloropropyl)sulfonyl)piperazin-2-one

Following General Step O, 1-(3-(4-chloro-3-(2,2-difluoroethyl)-1H-pyrrolo[2,3-b]pyridin-5-yl)phenyl)-4-((3-chloropropyl)sulfonyl)piperazin-2-one was prepared as a yellow solid (130 mg, 55%). Mass (m/z): 531.1 [M+H]⁺.

### Step 2. Preparation of 1-(3-(4-chloro-3-(2,2-difluoroethyl)-1H-pyrrolo[2,3-b]pyridin-5-yl)phenyl)-4-((3-morpholinopropyl)sulfonyl)piperazin-2-one

Following General Step P, 1-(3-(4-chloro-3-(2,2-difluoroethyl)-1H-pyrrolo[2,3-b]pyridin-5-yl)phenyl)-4-((3-morpholinopropyl)sulfonyl)piperazin-2-one was prepared as a pale yellow solid (55 mg, 13%). Mass (m/z): 581.8 [M+H]⁺.

### Example 60

### 1-(3-(4-chloro-3-(2,2-difluoroethyl)-1H-pyrrolo[2,3-b]pyridin-5-yl)phenyl)-4-((3-(dimethylamino)propyl)sulfonyl)piperazin-2-one

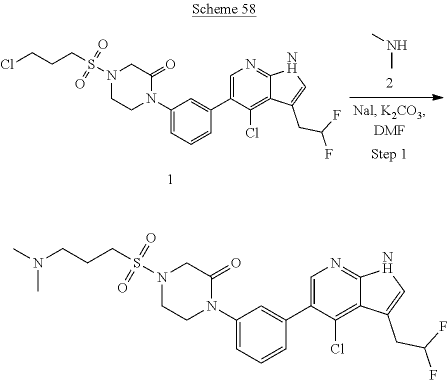

### Step 1. 1-(3-(4-chloro-3-(2,2-difluoroethyl)-1H-pyrrolo[2,3-b]pyridin-5-yl)phenyl)-4-((3-(dimethylamino)propyl)sulfonyl)piperazin-2-one

Following General Step P, 1-(3-(4-chloro-3-(2,2-difluoroethyl)-1H-pyrrolo[2,3-b]pyridin-5-yl)phenyl)-4-((3-(dimethylamino)propyl)sulfonyl)piperazin-2-one was prepared as a white solid (1.2 g, 65%). Mass (m/z): 540.2 [M+H]⁺.

### Example 61

### 1-(6-(4-chloro-3-(2,2-difluoroethyl)-1H-pyrrolo[2,3-b]pyridin-5-yl)pyridin-2-yl)-4-(methylsulfonyl)piperazin-2-one

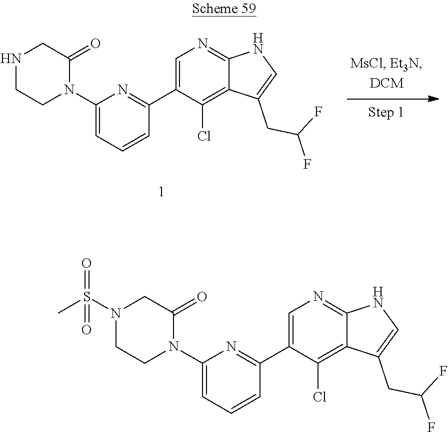 [SMILES: O=C1CNCCN1c1cccc(-c2cnc3[nH]cc(CC(F)F)c3c2Cl)n1 ; CS(=O)(=O)N1CCN(c2cccc(-c3cnc4[nH]cc(CC(F)F)c4c3Cl)n2)C(=O)C1]

### Step 1. Preparation of 1-(6-(4-chloro-3-(2,2-difluoroethyl)-1H-pyrrolo[2,3-b]pyridin-5-yl)pyridin-2-yl)-4-(methylsulfonyl)piperazin-2-one

Following General Step O, 1-(6-(4-chloro-3-(2,2-difluoroethyl)-1H-pyrrolo[2,3-b]pyridin-5-yl)pyridin-2-yl)-4-(methylsulfonyl)piperazin-2-one was prepared as a light yellow solid (3 mg, 7.4%). Mass (m/z): 470.1 [M+H]⁺.

### Example 62

### 1-(3-(4-chloro-3-(2,2-difluoroethyl)-1H-pyrrolo[2,3-b]pyridin-5-yl)phenyl)-4-((2-methoxyethyl)sulfonyl)piperazin-2-one

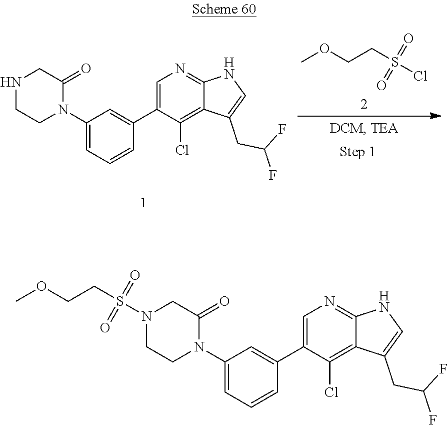

### Step 1. Preparation of 1-(3-(4-chloro-3-(2,2-difluoroethyl)-1H-pyrrolo[2,3-b]pyridin-5-yl)phenyl)-4-((2-methoxyethyl)sulfonyl)piperazin-2-one

Following General Step O, 1-(3-(4-chloro-3-(2,2-difluoroethyl)-1H-pyrrolo[2,3-b]pyridin-5-yl)phenyl)-4-((2-methoxyethyl)sulfonyl)piperazin-2-one was prepared as a yellow solid (15 mg, 7.3%). Mass (m/z): 513.1 [M+H]⁺.

### General Assay Procedures:

### HPK1 Enzyme Inhibition Assay

The compound was dissolved in 100% DMSO at the concentration of 10 mM. The HPK1 protein was purchased from Signal Chem (M23-11G-10). 2.5 μL per well of 2×HPK1 protein was added to assay plate containing the test compound, centrifuged at 1500 rpm for 1 minute, and then incubated at 25° C. for 60 minutes. MBP protein was purchased from Signal Chem (M42-51N) and ATP was purchased from Promega (V9102). The two were added 2.5 μL per well mixture of 2×MBP (0.2 ug/ul) and ATP (20 μM), centrifuged at 1500 rpm for 1 minute, then incubated at 25° C. for 60 minutes. Then added 5 μL of ADP-Glo from Promega (V9102) to the assay plate and depleted the unconsumed ATP for 60 minutes. Then centrifuged at 1500 rpm for 1 minute and incubated at 25° C. for 60 minutes. Finally, 10 μL of the kinase assay reagent from Promega (V9102) was added to the assay plate to convert ADP to ATP, centrifuged at 1500 rpm for 1 minute, incubate at 25° C. for 40 minutes. After 40 minutes incubation, the fluorescence was determined. Based on the results, the IC₅₀ value of the compound was calculated. The results of IC₅₀ are shown in the following Table 2.

+++: IC₅₀<=10 nM; ++: 10 nM<IC₅₀<=100 nM; +: 100 nM<IC₅₀<=1 μM; NA: not active, IC₅₀>1 μM

TABLE 2
Results of compounds in HPK1
enzyme inhibition assays

| | HPK1 enzyme |
|---|---|
| Example | inhibition IC₅₀ |
| 1 | +++ |
| 2 | + |
| 3 | + |
| 4 | +++ |
| 5 | +++ |
| 6 | ++ |
| 7 | ++ |
| 8 | NA |
| 9 | +++ |
| 10 | ++ |
| 11 | ++ |
| 12 | +++ |
| 13 | ++ |
| 14 | ++ |
| 15 | +++ |
| 16 | ++ |
| 17 | ++ |
| 18 | ++ |
| 19 | + |
| 20 | + |
| 21 | NA |
| 22 | +++ |
| 23 | +++ |
| 24 | +++ |
| 25 | +++ |
| 26 | +++ |
| 27 | +++ |
| 28 | +++ |
| 29 | +++ |
| 30 | ++ |
| 31 | +++ |
| 32 | +++ |
| 33 | ++ |
| 34 | ++ |
| 35 | ++ |
| 36 | ++ |
| 37 | ++ |
| 38 | +++ |
| 39 | +++ |
| 40 | NA |
| 41 | NA |
| 42 | ++ |
| 43 | +++ |
| 44 | +++ |
| 45 | +++ |
| 46 | +++ |
| 47 | ++ |
| 48 | +++ |
| 49 | +++ |
| 50 | +++ |
| 51 | +++ |
| 52 | +++ |
| 53 | +++ |
| 54 | +++ |
| 55 | +++ |
| 56 | +++ |
| 57 | +++ |
| 58 | +++ |
| 59 | +++ |
| 60 | +++ |
| 61 | +++ |
| 62 | +++ |

HPK1 p-SLP-76 Inhibition Assay

Before compound treatment, Jurkat cells were starved in PRMI-1640 medium supplemented with 0.5% FBS overnight. The cell density was adjusted to 1*10\hat{ }7 cells/mL in assay medium (RPMI-1640 no phenol red with 0.5% FBS) and 16 uL cell suspension was transferred per well to a 384-well plate (Corning #3765). 6× solution of test compound was prepared in assay medium and 4 uL was added per well to the 384-well plate. The plate was then incubated at 37° C., 5% CO₂ for 4 h. Jurkat cells in 384-well plate were stimulated with anti-CD3 (BD Biosciences #555329, final conc. is 10 ug/mL) for 20 min at 37° C., 5% CO₂ by adding 4 uL 6× antibody solution per well. Phospho-SLP76 HTRF kit was purchased from Cisbio (63ADK076PEH). 8 uL 4× Lysis Buffer was added to 384-well plate after anti-CD3 stimulation. The plate was centrifuged at 1,000 rpm for 1 min, shaken at 250 rpm for 60 min and centrifuged again at 1,000 rpm for 5 min. Afterwards the cell lysates were transferred to the assay plate (Greiner #784075) with 16 uL per well. 4 uL detection antibody mix per well prepared in Detection Buffer was added to the assay plate, followed by centrifugation at 1,000 rpm for 1 min and incubation at 25° C. overnight. The fluorescence emission at two different wavelengths (665 nm and 620 nm) was read by an HTRF compatible microplate reader and the IC₅₀ value of the compound was calculated.

A: IC₅₀<=300 nM; B: 300 nM<IC₅₀<=3 μM; C: 3 μM<IC₅₀<=10 μM; NA: not active, IC₅₀>10 μM

TABLE 3
Results of compounds inhibition of
HPK1 p-SLP-76 in Jurkat cells

| Example | p-SLP-76 inhibition IC₅₀ |
|---|---|
| 1 | B |
| 2 | NA |
| 3 | B |
| 4 | B |
| 5 | NA |
| 6 | B |
| 7 | C |
| 8 | NA |
| 9 | B |
| 10 | B |
| 11 | C |
| 12 | B |
| 13 | C |
| 14 | NA |
| 15 | A |
| 16 | B |
| 17 | B |
| 18 | C |
| 19 | NA |
| 20 | NA |
| 21 | NA |
| 22 | B |
| 23 | B |
| 24 | B |
| 25 | A |
| 26 | B |
| 27 | A |
| 28 | B |
| 29 | B |
| 30 | B |
| 31 | B |
| 32 | A |
| 33 | C |
| 34 | C |
| 35 | C |
| 36 | NA |
| 37 | B |
| 38 | B |
| 39 | B |
| 40 | NA |
| 41 | NA |
| 42 | C |
| 43 | C |
| 44 | B |
| 45 | NA |
| 46 | C |
| 47 | NA |
| 48 | NA |
| 49 | NA |
| 50 | B |
| 51 | B |
| 52 | B |
| 53 | B |
| 54 | B |
| 55 | B |
| 56 | B |
| 57 | B |
| 58 | B |
| 59 | B |
| 60 | B |
| 61 | B |
| 62 | B |

### FLT3-ITD Enzyme Inhibition Assay

The compound was dissolved in 100% DMSO at the concentration of 10 mM. The FLT3-ITD protein was purchased from Invitrogen (PV6190). 5 μL per well of 2×FLT3-ITD protein was added to assay plate containing the test compound, centrifuged at 1000 rpm for 1 minute, and then incubated at 25° C. for 15 minutes. HTRF KinEASE-TK kit was purchased from Cisbio (62TK0PEC) and ATP was purchased from Thermo Fisher Scientific (R0441). TK-Substrate-biotin and ATP were added 5 μL per well mixture of 2×TK-Substrate-biotin (final conc. is 2 M) and ATP (final conc. is 15 μM), centrifuged at 1000 rpm for 1 minute, then incubated at 25° C. for 60 minutes. Then added 10 μL of detection buffer (0.25×TK-antibody Eu3+-cryptate, 0.125 μM streptavidine-XL 665) to the assay plate and centrifuged at 1000 rpm for 1 minute then incubated at 25° C. for 60 minutes and 4° C. incubated for overnight, the product was determined. Based on the results, the IC₅₀ value of the compound was calculated. The results of IC₅₀ are shown in Table 3.

+++: IC₅₀<=10 nM; ++: 10 nM<IC₅₀<=100 nM; +: 100 nM<IC₅₀<=1 μM; NA: not active, IC₅₀>1 μM

TABLE 3
Results of selected compounds in
FLT3-ITD enzyme inhibition assay

| | FLT3 Enzyme |
|---|---|
| Example | inhibition IC₅₀ |
| 7 | ++ |
| 20 | ++ |
| 25 | +++ |
| 43 | ++ |
| 44 | +++ |
| 45 | +++ |
| 48 | +++ |
| 50 | +++ |
| 53 | +++ |
| 60 | +++ |

### Other Embodiments

The present disclosure provides merely exemplary embodiments. One skilled in the art will readily recognize from the present disclosure and claims, that various changes, modifications and variations can be made therein without departing from the spirit and scope of the present disclosure as defined in the following claims.

1. A compound of Formula (I): 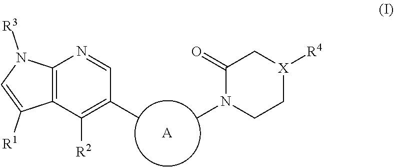
a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) R² is chosen from hydrogen, halogen groups, OR^{x}, SR^{x}, NHR^{x}, N(R^{x})₂, CHR^{x}, and C(R^{x})₂;
(iii) R³ is chosen from hydrogen, linear, branched, and cyclic alkyl groups, heterocyclic groups, aryl groups, and heteroaryl groups;
(iv) R⁴ is chosen from hydrogen, linear, branched, and cyclic alkyl groups, heterocyclic groups, C(O)R^{y}, CO₂R^{y}, C(O)R^{w}OR^{y}, C(O)R^{w}N(R^{x}R^{y})₂, OC(O)R^{w}NR^{x}R^{y}, S(O)R^{y}, and SO₂R^{y};
(v) X is chosen from N and CR^{x};
(vi) each R^{x} and R^{y} is independently chosen from hydrogen, linear, branched, and cyclic alkyl groups, carbocyclic groups, heterocyclic groups, aryl groups, and heteroaryl groups;
(vii) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(viii) ring A is chosen from optionally substituted aryls, optionally substituted heteroaryls, optionally substituted cycloalkyls, and optionally substituted heterocycloalkyls;
wherein the linear, branched, and cyclic alkyl groups, linear, branched, and cyclic alkenyl groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups optionally substituted with 0, 1, or 2 C₁-C₆ linear, branched, and cyclic alkyl groups.

2. A compound of Formula (I′): 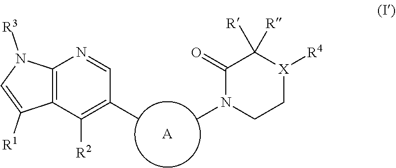
a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) R² is chosen from hydrogen, halogen groups, OR^{x}, SR^{x}, NHR^{x}, N(R^{x})₂, CHR^{x}, and C(R^{x})₂;
(iii) R³ is chosen from hydrogen, linear, branched, and cyclic alkyl groups, heterocyclic groups, aryl groups, and heteroaryl groups;
(iv) R⁴ is chosen from hydrogen, linear, branched, and cyclic alkyl groups, heterocyclic groups, C(O)R^{y}, CO₂R^{y}, C(O)R^{w}OR^{y}, C(O)R^{w}NC(O)R^{x}, C(O)R^{w}NC(O)R^{x}NHR^{y}, C(O)R^{w}NR^{x}R^{y}, OC(O)R^{w}NR^{x}R^{y}, S(O)R^{y}, and SO₂R^{y};
(v) each R′ and R″ is independently chosen from hydrogen, linear, branched, and cyclic alkyl groups, linear, branched, and cyclic aminoalkyl groups, carbocyclic groups, heterocyclic groups;
(vi) X is chosen from N and CR^{x};
(vii) each R^{x} and R^{y} is independently chosen from hydrogen, linear, branched, and cyclic alkyl groups, linear, branched, and cyclic aminoalkyl groups, carbocyclic groups, heterocyclic groups, aryl groups, heteroaryl groups, and a glycosidic group;
(viii) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(ix) ring A is chosen from optionally substituted aryls, optionally substituted heteroaryls, optionally substituted cycloalkyls, and optionally substituted heterocycloalkyls;
wherein the linear, branched, and cyclic alkyl groups, linear, branched, and cyclic alkenyl groups, carbocyclic groups, linear, branched, and cyclic aminoalkyl groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
carboxylate group;
hydroxy,
thiol,
amino,
cyano, 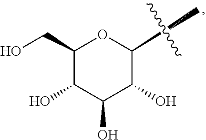
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic aminoalkylcarboxylate groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups optionally substituted with 0, 1, or 2 C₁-C₆ linear, branched, and cyclic alkyl groups or halogen groups, and
5 and 6-membered heteroaryl groups optionally substituted with 0, 1, or 2 C₁-C₆ linear, branched, and cyclic alkyl groups.

3. A compound of Formula (IIA): 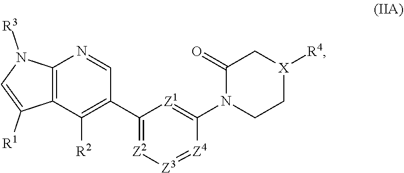
a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) R² is chosen from hydrogen, halogen groups, OR^{x}, SR^{x}, NHR^{x}, N(R^{x})₂, CHR^{x}, and C(R^{x})₂;
(iii) R³ is chosen from hydrogen, linear, branched, and cyclic alkyl groups, heterocyclic groups, aryl groups, and heteroaryl groups;
(iv) R⁴ is chosen from hydrogen, linear, branched, and cyclic alkyl groups, heterocyclic groups, C(O)R^{y}, CO₂R^{y}, C(O)R^{w}OR^{y}, C(O)R^{w}N(R^{x}R^{y})₂, OC(O)R^{w}NR^{x}R^{y}, S(O)R^{y}, and SO₂R^{y};
(v) X is chosen from N and CR^{x};
(vi) each Z¹, Z², Z³, and Z⁴ is independently chosen from CR^{z} or N;
(vii) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(viii) each R^{x}, R^{y}, and R^{z} is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups optionally substituted with 0, 1, or 2 C₁-C₆ linear, branched, and cyclic alkyl groups.

4. A compound of Formula (IIIA): 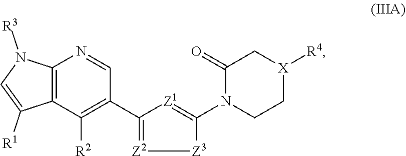
a tautomer thereof, a deuterated derivative of the compound or the tautomer, or a pharmaceutically acceptable salt the foregoing, wherein:
(x) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(xi) R² is chosen from hydrogen, halogen groups, OR^{x}, SR^{x}, NHR^{x}, N(R^{x})₂, CHR^{x}, and C(R^{x})₂;
(xii) R³ is chosen from hydrogen, linear, branched, and cyclic alkyl groups, heterocyclic groups, aryl groups, and heteroaryl groups;
(xiii) R⁴ is chosen from hydrogen, linear, branched, and cyclic alkyl groups, heterocyclic groups, C(O)R^{y}, CO₂R^{y}, C(O)R^{w}OR^{y}, C(O)R^{w}N(R^{x}R^{y})₂, OC(O)R^{w}NR^{x}R^{y}, S(O)R^{y}, and SO₂R^{y};
(xiv) X is chosen from N and CR^{x};
(xv) each Z¹ and Z² is independently chosen from CR^{z} or N;
(xvi) Z³ is chosen from O, S, and NW; and
(xvii) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(xviii) each R^{x}, R^{y}, and R^{z} is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups optionally substituted with 0, 1, or 2 C₁-C₆ linear, branched, and cyclic alkyl groups.

5. A compound of Formula (IIIB): 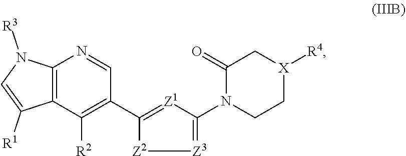
a tautomer thereof, a deuterated derivative of the compound or the tautomer, or a pharmaceutically acceptable salt the foregoing, wherein:
(x) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(xi) R² is chosen from hydrogen, halogen groups, OR^{x}, SR^{x}, NHR^{x}, N(R^{x})₂, CHR^{x}, and C(R^{x})₂;
(xii) R³ is chosen from hydrogen, linear, branched, and cyclic alkyl groups, heterocyclic groups, aryl groups, and heteroaryl groups;
(xiii) R⁴ is chosen from hydrogen, linear, branched, and cyclic alkyl groups, heterocyclic groups, C(O)R^{y}, CO₂R^{y}, C(O)R^{w}OR^{y}, C(O)R^{w}N(R^{x}R^{y})₂, OC(O)R^{w}NR^{x}R^{y}, S(O)R^{y}, and SO₂R^{y};
(xiv) X is chosen from N and CR^{x};
(xv) each Z¹ and Z³ is independently chosen from CR^{z} or N;
(xvi) Z² is chosen from O, S, and NW;
(xvii) R^{w} is absent or is chosen from, linear, branched, and cyclic alkyl groups, carbocyclic groups, heterocyclic groups, linear, branched, and cyclic alkenyl groups, linear and branched heteroalkenyl groups; and
(xviii) each R^{x}, R^{y}, and R^{z} is independently chosen from hydrogen, linear, branched, and cyclic alkyl groups, carbocyclic groups, heterocyclic groups, aryl groups, and heteroaryl groups;
wherein the linear, branched, and cyclic alkyl groups, linear, branched, and cyclic alkenyl groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups optionally substituted with 0, 1, or 2 C₁-C₆ linear, branched, and cyclic alkyl groups.

6. A compound chosen from 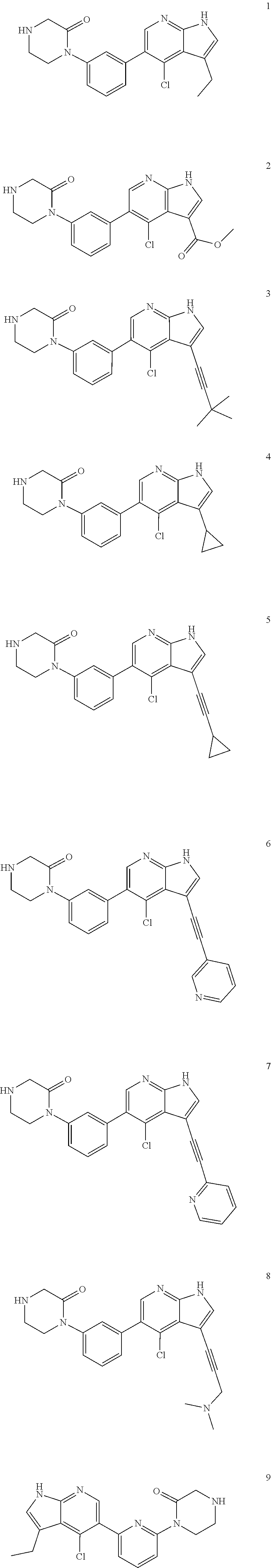 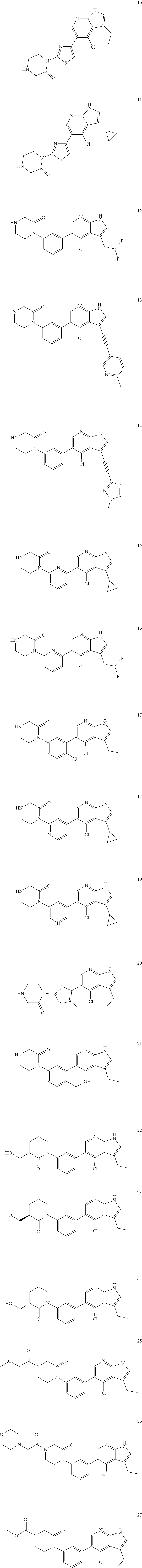 [chemical structure] 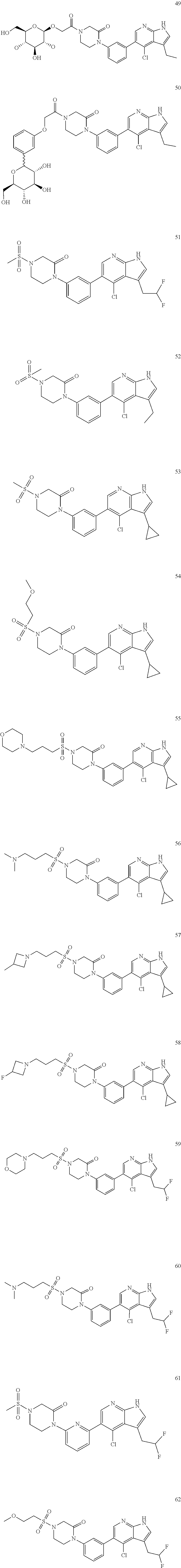
a tautomer thereof, a deuterated derivative of the compound or the tautomer, or a pharmaceutically acceptable salt of the foregoing.

7. A pharmaceutical composition comprising a compound, tautomer, deuterated derivative, or pharmaceutically acceptable salt according to any one of claims 1-6 and at least one pharmaceutically acceptable carrier.

8. A method for treating or alleviating a disease, a disorder or a condition mediated by the inhibition of hematopoietic progenitor kinase 1 (HPK1) and/or Human Fms-like tyrosine kinase 3 (FLT3), comprising administering to a subject in need thereof a therapeutically effective amount of a compound, tautomer, deuterated derivative, or pharmaceutically acceptable salt according to any one of the claims 1-6 or the pharmaceutical composition according to claim 7.

9. A method for decreasing HPK1 and/or FLT3 activity in a disease, a disorder or a condition, comprising administering to a subject in need thereof a therapeutically effective amount of a compound, tautomer, deuterated derivative, or pharmaceutically acceptable salt according to any one of the claims 1-6 or the pharmaceutical composition according to claim 7.

10. The method of claim 9, wherein the disease, the disorder, or the condition is chosen from an HPK1 and/or FLT3-related disease; wherein the HPK1 and/or FLT3-related disease comprises cancer, a dysregulated immune response, or a disease involved in aberrant HPK1 and/or FLT3 expression, activity, and/or signaling.

11. The method of claim 10, wherein the cancer is chosen from brain cancer, breast cancer, respiratory tract and/or lung cancer, a reproductive organ cancer, bone cancer, digestive tract cancer, urinary tract cancer, eye cancer, liver cancer, kidney cancer, skin cancer, head and neck cancer, anal cancer, nervous system cancer, thyroid cancer, parathyroid cancer, a lymphoma, a sarcoma, and a leukemia.
